(19) United States
(12) Patent Application Publication
(10) Pub. No.: US 2025/0218551 A1
(43) Pub. Date: Jul. 3, 2025

(54) GENERATIVE ATOMISTIC DESIGN OF MATERIALS

(71) Applicant: Quantum Generative Materials LLC, Austin, TX (US)

(72) Inventors: Jacob Vikoren, Pacific Beach, WA (US); Deeptanshu Prasad, Seattle, WA (US); David Sommer, Duvall, WA (US); Vaidish Sumaria, San Jose, CA (US)

(21) Appl. No.: 18/397,993

(22) Filed: Dec. 27, 2023

(51) Int. Cl. G16C 20/30; G16C 20/70

(52) U.S. Cl. CPC G16C 20/30; G16C 20/70; G16C 60/00; G06F 30/27; G06N 20/10

(57) ABSTRACT

A system and method are provided for generative atomistic design of materials. The disclosure herein includes a machine learning system for generating a new material, using multiple predictive machine learning models for atomic level properties to create training data, and/or then using the same predictive machine learning models to refine the output of a generative machine learning system. In use, one or more datasets are received at at least one computing device corresponding to a desired material. Additionally, using at least two machine learning models associated with the at least one computing device, a new dataset is created for the desired material. Further, the at least two machine learning models are trained, using at least semi-supervised learning, based on the one or more datasets, to model properties of the desired material. Still yet, using the at least one computing device, a prediction is outputted comprising the desired material.

### FIELD OF THE INVENTION

The present invention relates to solid-state materials, and more particularly to the generation of novel inorganic solid-state materials.

### BACKGROUND

Currently, generating novel materials that fulfill specific needs involves extremely time-consuming efforts that often involve running a number of experiments that are designed based on expert knowledge, and then refining those experiments and adjusting those experimental parameters in order to approach a more optimal solution and outcome. The outcome in question is a physical material that retains the properties of interest, but which must undergo many cycles of experimentation and evaluation to determine the resulting material's level of compliance and utility. As such, previous efforts to perform advanced materials research and development are typically slow and lead to protracted development cycles. Such efforts are typically siloed across organizations and infrastructures, which can increase friction and fail to capitalize on possible synergies. In addition, such efforts inherently bring with them greater data generation demands and higher computational costs. Further, such efforts are limited in the complexity and scale of systems that they can address.

As such, there is thus a need for addressing these and/or other issues associated with the prior art.

### SUMMARY

A system and method are provided for generative atomistic design of materials. The disclosure herein includes a machine learning system for generating a new material, using multiple predictive machine learning models for atomic level properties to create training data, and then using the same predictive machine learning models to refine the output of a generative machine learning system. In use, one or more datasets are received at at least one computing device corresponding to a desired material. Additionally, using at least two machine learning models associated with the at least one computing device, a new dataset is created for the desired material. Further, the at least two machine learning models are trained, using at least semi-supervised learning, based on the one or more datasets, to model properties of the desired material. Still yet, using the at least one computing device, a prediction is outputted comprising the desired material.

In some aspects, the techniques described herein relate to a method, including: receiving, at at least one computing device, one or more datasets corresponding to a desired material; creating, using at least two machine learning models associated with the at least one computing device, a new dataset for the desired material, wherein the at least two machine learning models are trained, using at least semi-supervised learning, based on the one or more datasets, to model properties of the desired material; and outputting, using the at least one computing device, a prediction including the desired material.

In some aspects, the techniques described herein relate to a method, wherein the at least two machine learning models include at least one of machine learning interatomic potential (MLIP), machine learned charge density (MLCD), or machine learning property predictor (MLProp).

In some aspects, the techniques described herein relate to a method, wherein the at least two machine learning models include at least two of machine learning interatomic potential (MLIP), machine learned charge density (MLCD), or machine learning property predictor (MLProp).

In some aspects, the techniques described herein relate to a method, wherein the at least two machine learning models include quantum data or Quantum Probabilistic Machine Learning.

In some aspects, the techniques described herein relate to a method, wherein the at least two machine learning models are predictive machine learning models.

In some aspects, the techniques described herein relate to a method, further including evaluating the desired material using uncertainty-driven active learning.

In some aspects, the techniques described herein relate to a method, further including: training two or more predictive machine learning models on the one or more datasets; using the trained predictive models to create a second larger dataset; generating, using a generative machine learning model trained on the larger dataset, a proposed atomic structure; refining the proposed atomic structure by adjusting the structure to maximize stability; evaluating the proposed atomic structure using uncertainty-driven active learning; determining that the proposed atomic structure is below an error threshold; and validating the proposed atomic structure via density functional theory. It is to be understood that a proposed atomic structure may include a single structure or a variety of structures.

In some aspects, the techniques described herein relate to a method, further including: outputting, using the at least one computing device, a generative model; and determining, using the at least two machine learning models associated with the at least one computing device, an accuracy of the generative model.

In some aspects, the techniques described herein relate to a method, wherein the generative model is outputted within an active learning computing environment.

In some aspects, the techniques described herein relate to a method, wherein the prediction is outputted based on the generative model.

In some aspects, the techniques described herein relate to a method, wherein the prediction includes iteratively reassessing the dataset for the desired material with the properties of the desired material until the properties of the desired material satisfies a predetermined threshold.

In some aspects, the techniques described herein relate to a method, wherein the prediction is outputted only when the generative model satisfies the predetermined threshold.

In some aspects, the techniques described herein relate to a method, wherein the prediction is outputted in fewer computing cycles compared to a conventional computation of the one or more datasets.

In some aspects, the techniques described herein relate to a method, wherein the desired material and properties of the desired material are received and analyzed using one or more Density Functional Theory (DFT) models.

In some aspects, the techniques described herein relate to a method, wherein at least one of: the creating integrates classical machine learning and quantum machine learning; the creating includes using experiment via Bayesian Optimization (BO) to predict the desired material; or the creating uses uncertainty-driven active learning cycles to create the new dataset for the desired material.

In some aspects, the techniques described herein relate to a method, wherein the new dataset for the desired material includes at least two of electronic properties, charge density, force vectors, interatomic potential, or a scalar value property associated with the desired material.

In some aspects, the techniques described herein relate to a method, further including synthesizing the desired material based on the prediction.

In some aspects, the techniques described herein relate to a method, wherein the at least semi-supervised learning uses uncertainty-driven active learning.

In some aspects, the techniques described herein relate to a method, wherein the at least two machine learning models are configured such that the prediction can be outputted in fewer processing cycles compared to conventional computing systems.

In some aspects, the techniques described herein relate to a method, wherein the at least two machine learning models are used in parallel.

In some aspects, the techniques described herein relate to a method, wherein the at least two machine learning models are used in serial.

In some aspects, the techniques described herein relate to a system, including: a non-transitory memory storing instructions; and one or more processors in communication with the non-transitory memory, wherein the one or more processors execute the instructions to: receive, at at least one computing device, one or more datasets corresponding to a desired material; create, using at least two machine learning models associated with the at least one computing device, a new dataset for the desired material, wherein the at least two machine learning models are trained, using at least semi-supervised learning, based on the one or more datasets, to model properties of the desired material; and output, using the at least one computing device, a prediction including the desired material.

In some aspects, the techniques described herein relate to a method, including: predicting, using at least one processor, using at least two machine learning models, a new material with desired properties by: predicting the new material using at least two machine learning models, wherein the at least two machine learning models are trained based on the desired properties; analyzing the prediction of the new material via the at least two machine learning models; and outputting the prediction once the prediction exceeds a predetermined threshold associated with the at least two machine learning models.

In some aspects, the techniques described herein relate to a method, including: creating an active learning cycle, using at least two machine learning models, by: generating training data based on property data, iteratively reprocessing the property data and the training data until a predetermined threshold is met, wherein the predetermined threshold includes a set of characteristics, and once the predetermined threshold is met, generating a predictive model; generating a machine learning model using the predictive model; and applying the machine learning model to predict a new material having the characteristics.

### BRIEF DESCRIPTION OF THE DRAWINGS

FIG. 1A illustrates an illustration for generative atomistic design, in accordance with one embodiment.

FIG. 1B illustrates a method for generating predictive models for material generation, in accordance with one embodiment.

FIG. 1C illustrates a system for reiterative model training with quantum capabilities, in accordance with one embodiment.

FIG. 2 illustrates a system for outputting one or more atomic structure datasets trained via classical and/or quantum machine learning, in accordance with one embodiment.

FIG. 3 illustrates a system for outputting trained and refined generative machine learning models for candidate atomic structures, in accordance with one embodiment.

FIG. 4 illustrates a system for refining trained machine learning models for candidate atomic structures, in accordance with one embodiment.

FIG. 5A illustrates a training architecture for material generation, in accordance with one embodiment.

FIG. 5B illustrates a generating architecture for material generation, in accordance with one embodiment.

FIG. 5C illustrates an alternative architecture for material generation, in accordance with one embodiment.

FIG. 5D illustrates an alternative architecture for material generation, in accordance with one embodiment.

FIG. 5E illustrates an alternative architecture for material generation, in accordance with one embodiment.

FIG. 5F illustrates an alternative architecture for material generation, in accordance with one embodiment.

FIG. 5G illustrates an alternative architecture for material generation, in accordance with one embodiment.

FIG. 6A illustrates a method for creating an active learning cycle using at least two machine learning models, in accordance with one embodiment.

FIG. 6B illustrates a method for predicting a new material with desired properties, in accordance with one embodiment.

FIG. 7A illustrates an environment in which various functions of the present disclosure may operate, in accordance with one embodiment.

FIG. 7B illustrates a dataset creation/loading and machine learning model training environment, in accordance with one embodiment.

FIG. 7C illustrates a prediction subsystem environment, in accordance with one embodiment.

FIG. 7D illustrates a generative training model subsystem environment, in accordance with one embodiment.

FIG. 7E illustrates a material selection and validation subsystem environment, in accordance with one embodiment.

FIG. 7F illustrates atomic structure conditions and environments, in accordance with one embodiment.

FIG. 7G illustrates a multi-layer perceptron environment, in accordance with one embodiment.

FIG. 7H illustrates a hardware and software architecture for a generative atomistic design (GAD) application, in accordance with one embodiment.

FIG. 7I illustrates a relationship between atomic structure training and inference data, in accordance with one embodiment.

FIG. 7J illustrates user and expert interactions with computing platforms, in accordance with one embodiment.

FIG. 8 illustrates a network architecture, in accordance with one possible embodiment.

FIG. 9 illustrates an exemplary system, in accordance with one embodiment.

FIG. 10 illustrates a system for active learning via constant model reevaluation and restructuring, in accordance with one embodiment.

FIG. 11 illustrates a collection of possible industrial benefits of novel atomic structure discovery through machine-learned modeling, in accordance with one embodiment.

### DETAILED DESCRIPTION

Currently, generating novel materials that fulfill specific needs involves extremely time-consuming efforts that often involve running a number of experiments that are designed based on expert knowledge, and then refining those experiments and adjusting those experimental parameters in order to approach a more optimal solution and outcome. For example, those researching in this space struggle with the vastness and complexity of the materials design space (due often to a multitude of variables influencing material properties). Additionally, materials science often requires expertise in various fields, making collaborative efforts essential for breakthroughs. Further issues are exacerbated through traditional trial-and-error approaches. Such approaches are often very resource-intensive and costly, impacting the feasibility of potential discoveries.

The outcome of such approach is a desired physical material that retains the properties of interest, but which must undergo many cycles of experimentation and evaluation to determine the resulting material's level of compliance and utility. As such, previous efforts to perform advanced materials research and development are typically slow and lead to protracted development cycles. Such efforts are typically siloed across organizations and infrastructures, which can increase friction and fail to capitalize on possible synergies. In addition, such efforts inherently bring with them greater data generation demands and higher computational costs. Further, such efforts are limited in the complexity and scale of systems that they can address.

Based on such known issues, many are turning to artificial intelligence (AI) for purposes of speeding up the research process for novel material generation, as well to drive down developmental costs. However, generating novel materials using AI faces several challenges that impact the effectiveness of the process, including issues of finding high quality (and availability) of materials data, issues with understanding the underlying principles governing materials behavior, and/or issues with high-cost barriers to involve an AI system.

In view of such considerations, the present disclosure introduces a generative atomistic design workflow for producing novel materials, including inorganic solid-state materials. The workflow integrates Density Functional Theory (DFT), Classical Machine Learning (CML), and/or Quantum Probabilistic Machine Learning (QPML). Further, the disclosure herein details a unique formulation that accelerates the research and optimization of known materials as well as the generative discovery of novel functional materials. By leveraging DFT and publicly available data, CML and QPML algorithms may be trained to predict material properties and dynamics. These computational models inform and are corroborated by experimental fabrication, synthesis, and characterization experiments.

Furthermore, the system is able to leverage uncertainty-driven active learning cycles to reduce the data requirements for training these models. Such a workflow decreases data generation requirements and extends the simulation capacity to accommodate larger systems and enables generative design of new material configurations with desired properties.

As such, the present disclosure represents a significant advancement in the field of novel material generation by effectively addressing and resolving known issues. Through innovative approaches and methodologies, the disclosure herein has successfully overcome challenges and limitations that have previously impeded progress in the art (noted hereinabove). These novel solutions contribute to a more comprehensive understanding material generation, and in particular, integration of AI systems for purposes of generating new materials.

As such, by harnessing the power of AI systems in materials discovery, almost all markets can benefit from optimized properties and functionalities tailored to specific needs. For example, in manufacturing, new materials can provide enhanced efficiency, durability, and cost-effectiveness, driving innovation in product design and production processes. The healthcare industry may benefit from personalized and precisely engineered materials for medical devices and drug delivery systems. The energy sector may allow for improved performance and efficiency for renewable energy technologies and energy storage. The foregoing examples are not to be limiting in any manner and are intended as representations of the vastness of applicability of the new generated materials. Therefore, the transformative impact of these new materials, generated by the generative atomistic design workflow disclosed herein, extends beyond specific industries (and in fact may apply to any and all industries), fostering a new era of material technological advancement, creation, personalization, and sustainability.

### Definitions and Use of Figures

Some of the terms used in this description are defined below for easy reference. The presented terms and their respective definitions are not rigidly restricted to these definitions-a term may be further defined by the term's use within this disclosure. The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion. As used in this application and the appended claims, the term “or” is intended to mean an inclusive “or” rather than an exclusive “or”. That is, unless specified otherwise, or is clear from the context, “X employs A or B” is intended to mean any of the natural inclusive permutations. That is, if X employs A, X employs B, or X employs both A and B, then “X employs A or B” is satisfied under any of the foregoing instances. As used herein, at least one of A or B means at least one of A, or at least one of B, or at least one of both A and B. In other words, this phrase is disjunctive. The articles “a” and “an” as used in this application and the appended claims should generally be construed to mean “one or more” unless specified otherwise or is clear from the context to be directed to a singular form.

Various embodiments are described herein with reference to the figures. It should be noted that the figures are not necessarily drawn to scale, and that elements of similar structures or functions are sometimes represented by like reference characters throughout the figures. It should also be noted that the figures are only intended to facilitate the description of the disclosed embodiments-they are not representative of an exhaustive treatment of all possible embodiments, and they are not intended to impute any limitation as to the scope of the claims. In addition, an illustrated embodiment need not portray all aspects or advantages of usage in any particular environment.

An aspect or an advantage described in conjunction with a particular embodiment is not necessarily limited to that embodiment and can be practiced in any other embodiments even if not so illustrated. References throughout this specification to “some embodiments” or “other embodiments” refer to a particular feature, structure, material or characteristic described in connection with the embodiments as being included in at least one embodiment. Thus, the appearance of the phrases “in some embodiments” or “in other embodiments” in various places throughout this specification are not necessarily referring to the same embodiment or embodiments. The disclosed embodiments are not intended to be limiting of the claims.

Within the context of the present description, atomic structure (or atomic configuration, material configuration) may refer to the arrangement of atoms in any matter. For example, the atomic structure may contain information about the atomic species and coordinates along with the lattice vectors which may used to specify Periodic Boundary Conditions (PBC). In various embodiments, understanding atomic structure may be foundational in materials science, as it may dictate a material's properties, behaviors, and functionalities. For example, the atomic structure may include various arrangements (such as a crystal lattice in metals or a more complex configuration in amorphous materials), which in turn may allow for the atomic structure to function as a blueprint from which all macroscopic material properties emerge.

Within the context of the present description, quantum probabilistic machine learning (QPML) may merge the principles of quantum mechanics, probabilistic machine learning, quantum computing and quantum computing inspired techniques. In various embodiments, QPML may harness the power of quantum computers or quantum computing simulators to process and analyze vast and complex datasets related to material properties and behaviors. Additionally, due to the inherent quantum nature of materials at the atomic and subatomic levels, QPML may provide unique advantages in understanding and predicting material properties' characteristics. As such, this fusion may allow for faster computations for certain tasks, as well as more accurate insight into material phenomena. As a result, QPML may potentially revolutionize the design and discovery of new materials and enhance the capabilities already realized within classical machine learning.

Within the context of the present description, computational materials science (CMS) may employ computer simulations for understanding material properties. For example, CMS may use calculations to understand, predict, and design material properties and behaviors. In an embodiment, advanced mathematical models and algorithms may simulate the manner in which atoms, molecules, and larger structures interact and behave under various conditions. As such, CMS may offer a more efficient path to gaining insight into materials via bypassing one or more time-intensive and costly experimental methods. It should be noted that, in one embodiment, CMS may provide a form of virtual laboratory, which may enable researchers to explore vast landscapes of material configurations, which may lead to innovations in industries ranging from electronics to energy to aerospace.

### Descriptions of Exemplary Embodiments

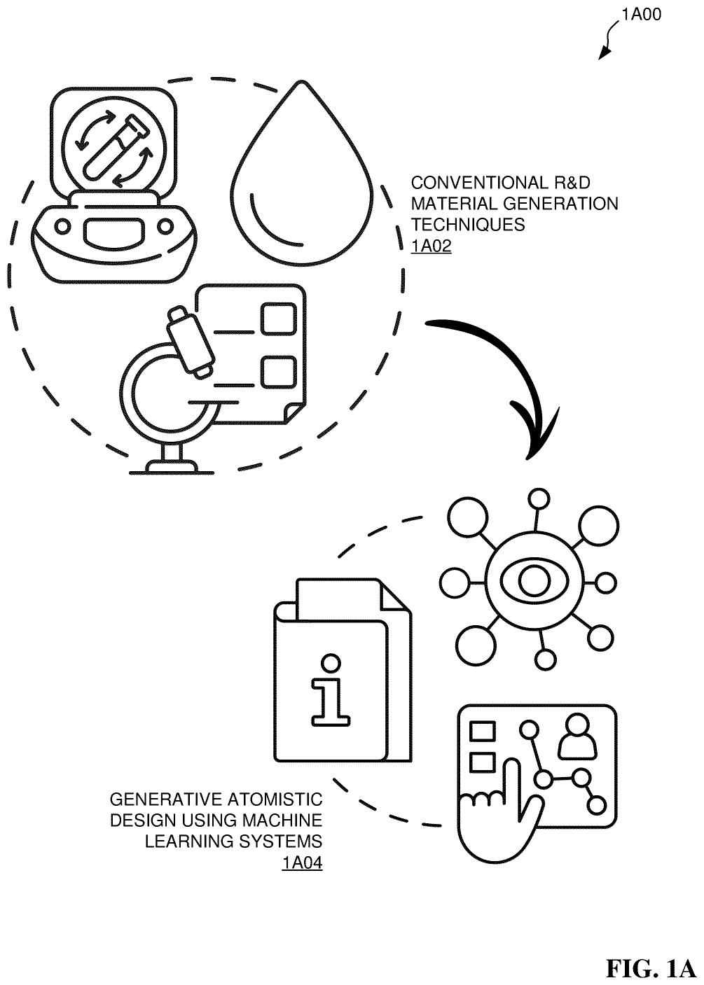

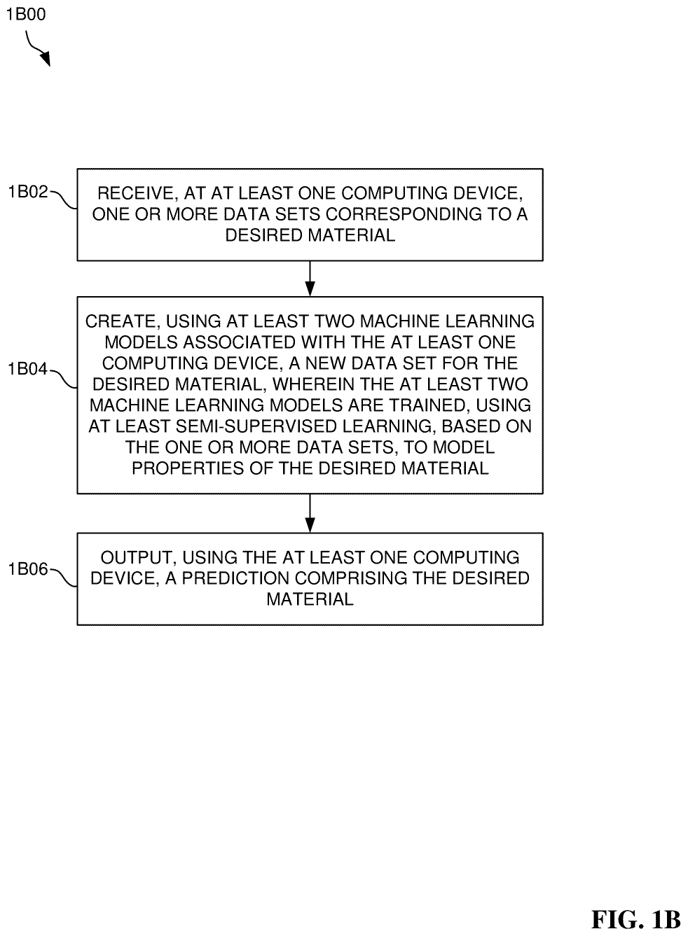

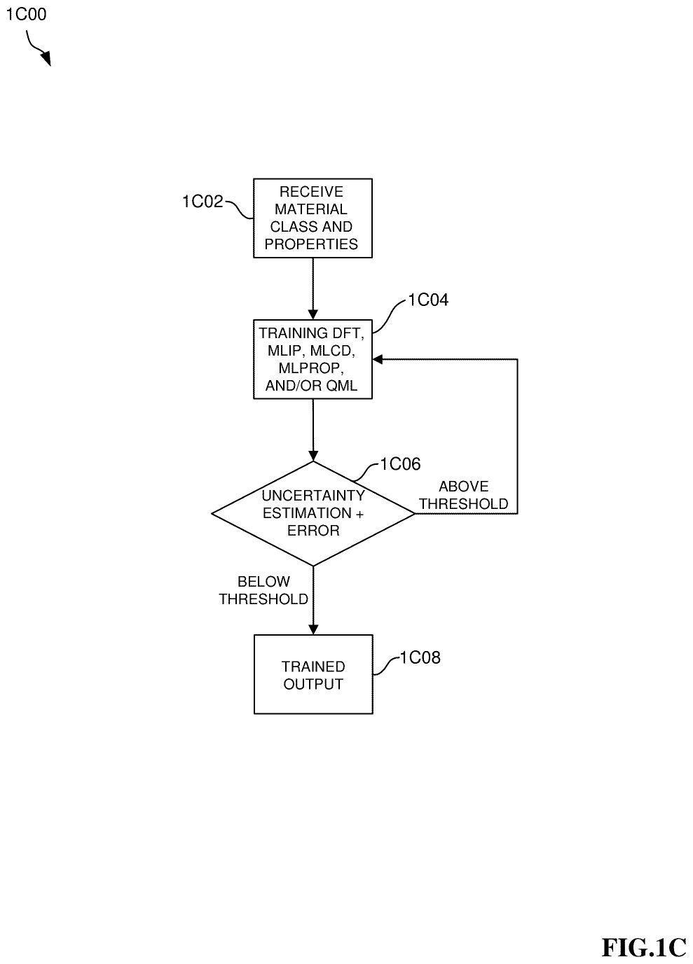

FIG. 1A illustrates an illustration 1A00 for generative atomistic design, in accordance with one embodiment. As an option, the illustration 1A00 may be implemented in the context of any one or more of the embodiments set forth in any previous and/or subsequent figure(s) and/or description thereof. Of course, however, the illustration 1A00 may be implemented in the context of any desired environment. Further, the aforementioned definitions may equally apply to the description below.

As shown, the illustration 1A00 includes conventional research and development material generation techniques 1A02. Such techniques may include a time-intensive approach to researching fundamental properties of materials, examining their composition, structure, and behavior under various conditions. However, like most forms of science, conventional research and development relies on key assumes, making a hypothesis, and then testing. Scientists often will craft educated guesses about potential material compositions, structures, or synthesis methods that could yield desired properties or functionalities. Subsequent tests may be used to validate or refute their conjectures.

Such techniques, however, include consistent and persistent challenges that impact its effectiveness and efficacy. For example, protracted timelines inherent in the iterative cycles of hypothesis formulation, experimentation, and analysis can impede the pace of innovation. High research costs, stemming from the need for specialized equipment and expertise, pose financial barriers, limiting the scope of projects and hindering broad accessibility. Additionally, the conventional techniques may inadvertently restrict the exploration of the expansive design space for materials, as researchers' preconceptions and biases may lead to the oversight of unconventional yet promising avenues.

Such known issues (and many more) may be resolved through generative atomistic design using machine learning systems 1A04. For example, by harnessing the power of advanced algorithms, machine learning expedites the testing of hypothesis structures, thereby navigating (in a much faster manner) the vast design space to propose novel materials with desirable properties. Moreover, the machine learning process optimize experimental design, efficiently suggesting conditions for material synthesis and characterization, minimizing resource-intensive trial-and-error approaches.

As such, machine learning may enable researchers to prioritize materials with high potential, streamlining the focus and allocation of resources. Further, machine learning systems may continuously adapt. For example, they may learn from training data to train them in a right way, which in turn, may allow the machine learning system to then generate additional data to improve predictions and recommendations. In essence, the integration of machine learning into materials R&D promises a paradigm shift, augmenting human capabilities and ushering in a more efficient, collaborative, and sustainable era in materials science.

In various embodiments, it is recognized that conventional experimental research can be expensive and time-intensive. The disclosure herein may allow for computer simulations for modeling and predicting material behavior in a manner that novel materials can be predicted, refined, outputted, and then synthesized.

With respect to the generative atomistic design using machine learning systems 1A04, it is to be appreciated that a number of potential architectures and flows may be used. That being said, the generative atomistic design using machine learning systems 1A04 may include, but not be limited to, material classes and properties that are specified (and which may be the basis for analysis by the machine learning system), and based on such trained information, the AI models and/or the machine learning models may be used to predict a resulting material conforming to the selected desired properties.

Further, in various embodiments, it is appreciated that the disclosure herein may include a generative machine learning system for generating a new material, using multiple predictive machine learning models for atomic level properties to create training data for a generative machine learning system, and then using the same training data to refine the output (i.e. generative materials) via a generative machine learning system. Additionally, it is to be understood that although the system is described as generating a new material (in the singular), any amount of new material (in the plural) may be generated through the process, system, and methods described in the present disclosure.

As such, as disclosed herein, the architecture used for the generative atomistic design using machine learning systems 1A04 may be used to predict generative materials. Such an architecture may include, but not be limited multiple predictive machine learning models used to predict the response, stability, properties, charge density, and/or forces of an atomic structure, an active learning cycle based on a generative machine learning model, and/or the use of quantum data (and Quantum Probabilistic Machine Learning) in the process of generating and/or refining atomic structures. As such, predictive machine learning, generative machine learning, quantum probabilistic machine learning, density function theory (DFT), experimentation, and active learning may be used in part or collectively to generate new atomic structures for materials.

More illustrative information will now be set forth regarding various optional architectures and uses in which the foregoing method may or may not be implemented, per the desires of the user/expert. It should be strongly noted that the following information is set forth for illustrative purposes and should not be construed as limiting in any manner. Any of the following features may be optionally incorporated with or without the exclusion of other features described. In particular, the foregoing illustration 1A00 may be shown in subsequent figures with a variety of different details, contexts and configurations.

FIG. 1B illustrates a method 1B00 for generating predictive models for material generation, in accordance with one embodiment. As an option, the method 1B00 may be implemented in the context of any one or more of the embodiments set forth in any previous and/or subsequent figure(s) and/or description thereof. Of course, however, the method 1B00 may be implemented in the context of any desired environment. Further, the aforementioned definitions may equally apply to the description below. As shown, the method 1B00 may receive, at at least one computing device, one or more datasets corresponding to a desired material. See operation 1B02. Within the context of the present application, a desired material may refer to any simulations of novel materials that fulfill specific needs and/or feature specific characteristics. For example, desired materials may comprise novel atomic structures designed for a variety of industrial uses in critical arenas such as, but not limited to, energy storage to advanced manufacturing. Further, such materials may include specialized creations (such as photovoltaics, semiconductors, battery electrolytes, etc.). Further descriptions relating thereto are provided in subsequent paragraphs. In particular, the description of the method 1B00 is intended to provide a detailed overview of the method and systems used to generate novel atomic structures. The architecture and systems (and exact flow of components) will be detailed in subsequent figures.

### Materials Dataset Creation/Loading

The one or more datasets may include a material dataset that is created/loaded for a desired material. In use, the one or more datasets may use an AI system, which may be leveraged to make accurate predictions regarding the properties that a specific material type may have. Such a system may significantly reduce the amount of time required in a physical lab to realize accurate simulations and accurate results. In one embodiment, the AI system may use a dataset creation/loading subsystem which may receive an initial dataset that may be taken in as initial phase input dataset. Such initial dataset may be in any appropriate format, and added to existing instances of input datasets. The updated instances of input datasets may then be used to load and/or create further datasets using one or more DFT-based tools for use in subsequent phases.

In one embodiment, the desired material and properties of the desired material may be received and analyzed using one or more Density Functional Theory (DFT) models. As such, DFT may be used as a computational approach for creating simulations of novel materials that fulfill specific needs and/or feature specific characteristics. For example, wherein a base level of testing may employ physical experimentation, an alternative method for determining atomic structure stability may employ computational methods within a DFT paradigm to help facilitate that process.

It is to be understood that DFT allows for computing properties based on atomic structure. Further, DFT may also function as a source of data such that, given an atomic structure, corresponding numbers may analyzed in DFT, and associated property data may be output, which in turn may then be passed to a machine learning model. Thus, the data driving the DFT subsystem may be based on synthetic and/or generated data that may otherwise be realized and recorded from an actual, physical experiment.

Contextually, the research and development process for creating novel materials that fulfill specific needs may be very time consuming and computationally intensive. As such, use of DFT processing may be faster than traditional physical development experimentation. For example, a materials developer may not have to (by way of practical example) order materials, wait a significant amount of time, get the desired materials into a lab, assign one or more team members to do the development and experimentation, perform routine material adjustments to the material during experimentation, and record measurements and results. Thus, DFT may prove significantly faster than traditional physical experimentation, as it is a simulation of actual physical experimentation. It should also be noted that DFT may require a lot of time and computing power to simulate the atomic structures. For example, by way of practical example, a typical DFT dataset may only measure around 10 GB, which may expand exponentially to arbitrary larger and larger sizes. As such, the methods disclosed herein assist with decreasing the amount of data that is tested using DFT.

Additionally, computational methods may (including DFT) rely on expert knowledge in a similar way to traditional development and experimental methods. A highly-trained experimentalist may have to decide which elements and/or compounds to put together as well as which actual material(s) to produce and physically test, and the same may be true in the computational domain of DFT. For example, in order to get useful and applicable information out of the DFT subsystem, there must still be an understandable starting point (i.e., the experimentalist must still start with a specific set of atoms and a particular configuration). Depending on the materials and associated properties brought into the generative atomistic design (GAD) application, such starting point may change, and the DFT computational method may learn something about what properties would result from that kind of system. As such, the set of atoms and particular configuration of the atoms may be configured as inputs into the DFT subsystem.

In one embodiment, DFT may assist in accelerating the process of predicting properties of atomic structures that are known, or that are provided to the DFT system. To this end, DFT may be used to create training data, such as for an AI system. Additionally, data may be collected from existing public repositories and used to train an AI system in order to learn resulting DFT-generated patterns. As such, DFT may be used to process computations so that the system may predict properties of materials that may not have previously been presented. Therefore, the DFT subsystem may be used to accelerate material(s) property prediction.

It is noted that predictive simulations via DFT processing may be very tiny, such as on the order of just a couple of hundred atoms. As such, part of the DFT subsystem process may be to extrapolate (or bridge) between two scales: a very tiny sample (perhaps only 200 atoms) and an exponentially larger atomic structure. By way of analogous example, an engineer may want to construct a bridge across an expanse. As a first step, the engineer may devise a scaled-down “model” for the ultimate large commercial structure intended to traverse a bridged area. In like manner, the experimental world is historically empirically macroscopic in that one may see large-scale results where each step in an atomic structure production may equate to a tiny block of material contributing to the larger structure.

Within a DFT framework, there may be different sub-approaches. For example, in a semiconductor system, a specific subdomain of DFT may be applied that is more accurate at predicting the properties of semiconductors. The known DFT framework for materials structure analysis has been developed by making direct comparisons between the different outputs from simulation and experimentation.

In one embodiment within the GAD application framework, the DFT subsystem may operate on a basic approximation of quantum mechanics in that matter is comprised of energy and waves that are distributed throughout space. As such, rather than trying to model every single electron, as well as all the associated waves of energy that may affect those electrons, DFT may analyze electron density at multiple points in space (hence the term “density functional theory”). Therefore, DFT may analyze points in space around a set of atoms and approximate electron density at those points in space. In an analogous embodiment, instead of a solar system-like relationship, where the nucleus is the sun and the electrons are planets rotating around, DFT may analyze the functional set (cloud) of energy waves like ripples on the on the surface of a lake. In further embodiment, DFT may be built based on the Schrodinger equation (built based on an approximation of the math), wherein the core benefit of DFT may be analyzing a known set of atomic types and locations, and approximating electron density around them.

As such, the DFT subsystem may be configured in a variety of ways to meet the needs of the material generation system.

In one embodiment, key assumptions may be relied upon in a DFT subsystem to speed up a development cycle. For example, if a Born-Oppenheimer approximation is used, DFT may treat the nucleus as a static particle and may focus processing cycles upon associated electron density information.

It is to be appreciated that, in the case of transition metal oxides, a DFT subsystem may be combined with quantum machine learning to successfully simulate atomic activity. For example, DFT is known to calculate a ground state accurately, but may not be designed predict excited states (which may play a key role in photovoltaics, semiconductors, and even battery electrolytes). Additionally, there may also be known electron systems outside the scope of typical DFT calculation capabilities. As such, in order to address these issues, quantum machine learning may be employed to assist DFT. Such a combination of DFT and quantum machine learning may assist, in one embodiment, with thermodynamic quantum statistical method derivation properties. For example, a gate drain source system may have a known issues of gate leakage. DFT, in combination with quantum machine learning, may be used to analyze such an issue to develop materials that would alleviate the gate leakage.

As discussed previously, in one embodiment, a DFT subsystem may be leveraged as a starting point for materials design. As such, proposed models may stem from a material that may comprise one or more properties including one or more particular values, and machine learning and/or AI systems may be used to propose a structure that might have those properties. Therefore, it should be noted that the DFT subsystem may introduce an additional step such as both property prediction as well as atomic structure generation. As such, this additional step may reduce the demand for specialized, expert knowledge because, as the space of possible materials may be so voluminous, materials datasets (which may measure in the millions) may not ever be fully explored when selecting a random number of structures. Thus, a spotlight approach used by the DFT subsystem may be employed to look at the range of all possibilities and highlight a starting structure that has a greater likelihood of success.

In one embodiment, the DFT subsystem may generate and output pairs of atomic structures and associated properties where the input may be the structure, and the output may be a prediction. In another embodiment, the DFT subsystem may generate pairs of atomic structures and associated properties that are used for training.

In a further embodiment, the DFT subsystem may provide information about the atomic structure and its charge density, which may be merely the position(s) of the electrons. As such, information regarding the atomic structure and its charge density may be used for gross properties analysis, where additional layers of quantum properties accessible via quantum probabilistic machine learning (QPML) may be introduced.

Within the context of the present description, charge density (CD) may refer to the distribution of electric charge. For example, CD may include a distribution of electric charge over a certain volume or surface (e.g., CD may measure how much electric charge is packed into a given space). In various embodiments, particularly when discussing atoms and molecules, charge density may provide insight into the electron distribution around atomic nuclei. As such, a detailed understanding of charge density may be crucial, as it may directly impact the material's electronic properties, chemical reactivity, and bonding characteristics. In practice, the analysis of charge density may provide researchers insight into the manner in which atoms and molecules interact, bond, and influence a larger material's properties.

Additionally, the method 1B00 may create, using at least two machine learning models associated with the at least one computing device, a new dataset for the desired material, wherein the at least two machine learning models are trained, using at least semi-supervised learning, based on the one or more datasets, to model properties of the desired material. See operation 1B04. In particular, the following paragraphs will detail how machine learning models and systems are used for training.

### Machine Learning

In use, a machine learning model training phase may be implemented using a machine learning model training subsystem controlled by a training manager that uses a training dataset (e.g., the output dataset from the DFT subsystem) to train one or more machine learning predictive material models.

In an applicable embodiment, the machine learning system may comprise three key parts, including receiving one or more desired properties for the atomic structure(s), employing multiple predictive systems that provide quantum properties, and using artificial intelligence (AI) models that may generate the one or more resulting materials that may encompass characteristics and criteria from the multiple predictive systems. As such, multiple predictive machine learning models may create training data for a single generative model in order to devise novel structures with the desired atomic structure properties.

In one embodiment, a machine learning model may be leveraged by analyzing a resulting generated structure, refining the structure using artificial intelligence via a method including predicting whether atoms within a structure need to adjust their relative positions to become more stable, and predicting the properties resulting from such stability. Additionally, the resulting generated structures may be filtered and passed back through DFT for verification (essentially performing a double-check by asking, “does DFT agree that that what machine learning suggested is actually true?”) Further, the machine learning process may also involve identifying and prioritizing conforming structures via experimentation and actual physical production to see whether, now that there may be actual materials upon which to experiment, the associated properties meet intended guidelines and threshold value(s).

For example, a typical material system may require 15,000 structures to be generated and passed through a machine learning model. In process, a subset of that data may be used for other models, where perhaps 15,000 structures may be required before the process begins to yield robust results. In such a data sample, a typical structure may focus on around 200 atoms (and/or any predetermined amount) within a single structure.

In one embodiment, machine learning models may draw on publicly-available atomic structure data repositories and leverage that atomic structure data to facilitate a machine learning workflow. In another embodiment, the collection of generated structures may be entirely internally generated, where all of the 15,000 (for example) structures may be created through a proprietary internal process, and thus not available elsewhere in the public domain. In addition, the overall process may include either a publicly-shared dataset or a proprietary (internally developed) dataset, and/or a combination of the two, depending on the atomic structures in question.

In another embodiment, the creating may integrate classical machine learning and quantum machine learning. For example, quantum data may be processed using classical machine learning on a classical computer, and quantum data may be processed using a quantum computing simulator on a quantum computer that uses a machine learning algorithm. In practice, classical machine learning and quantum machine learning models may employ the processing power of one or more quantum computers applied to classical data. Alternatively, quantum computers may be incorporated using quantum machine learning to calculate basic properties including thermodynamic properties and magnetic properties of much, much smaller systems than may be found in DFT systems. In a related embodiment, quantum computing simulators may be used instead of, or in addition to, actual quantum computing hardware.

It is to be appreciated that quantum data may relate to the behavior of matter and energy at the smallest scales, typically at the level of atoms and subatomic particles. As such, within the context of the present description, the machine learning models may use quantum computers for processing, but may also use and/or integrate quantum data for greater details on atomic behavior.

In one embodiment, the creating of the models may use uncertainty-driven active learning cycles to create the new dataset for the desired material. For example, the GAD application model may comprise an instance of an error computing module, which is a part of the active learning framework wherein model errors and uncertainty estimates are computed at the end of a training phase and/or a training epoch/regime, and a decision is made as to whether or not a newly evaluated structure must be recomputed with DFT. As such, if the errors and uncertainties exceed a selected threshold, the structure may be passed back to the DFT subsystem. This feedback mechanism may be used to enhance the predictive machine learning models (e.g., models in the form of one or more atomic structures, models in the form of periodic boundary conditions, etc.). Further, this feedback mechanism may also be used to enhance or confirm any one or more corresponding/associated global energy values, corresponding/associated interatomic force models, corresponding/associated charge density values, and/or corresponding global property values.

Within the context of the present description, uncertainty estimation (UE) in machine learning may quantify the confidence or reliability of a model's predictions. Additionally, rather than just offering a prediction, uncertainty estimation may also signal a level of certainty with regard to that prediction. As such, knowing the bounds and level of confidence may guide experimental efforts (such as by the machine learning system and/or models, etc.) when predicting the properties of a novel material. It should be noted that uncertainty estimation may play a pivotal role in active learning by quantifying a model's uncertainty with regard to one or more specific data points. Additionally, active learning may identify which samples in a collection might be most informative to label next, which may ensure that experimental resources are allocated most effectively. In notable embodiments, combining uncertainty estimation with active learning may significantly accelerate candidate materials' discovery and validation.

In one embodiment, the semi-supervised learning may combine the strengths of both labeled and unlabeled data to enhance model performance. For example, labeled data may include known outputs from DFT, and unlabeled data may include AI system generated data. As such, the use of semi-supervised learning may allow the machine learning models to exploit the information embedded in the unlabeled data (new potential structures), facilitating improved generalization to new, unseen instances of material generation. As such, the machine learning models may be trained specifically on the datasets originating initially from DFT.

In one embodiment, the at least semi-supervised learning may use uncertainty-driven active learning and predictive machine learning. It is noted that predictive machine learning may be a subset of machine learning (e.g., supervised machine learning) where machine learning models (e.g., machine-learned surrogate models) may be trained to make predictions about future or unseen data based on patterns identified from historical or known data. In some embodiments, criteria for training convergence may be set even when, after the learning rate is sufficiently low, no further improvement in the error may be observed. This process may be continued until the computed error, including uncertainty estimates, breaches a specified threshold. As such, one method of determining model error may be the use of a mean-square error computation and minimize the computed mean-square error between a predicted value from a predictive model and an expected value obtained from the determined ground truth. Typically, model uncertainties may be computed in terms of mean and standard deviation computations, and a chi-squared distribution may be used to compute a goodness value associated with the model uncertainties to compare with a threshold. In addition, it is envisioned that uncertainty estimates may be obtained using other statistical methods.

Machine learning algorithms learn to do what they do from data that they receive. As such, when high quality data forms the basis for machine learning analysis, the result may reliably be a high-quality model. It should be noted, however, that the performance of a machine learning model may be fundamentally limited by the quality of its data.

In an embodiment, the machine learning models may include a variety of models, including but not limited to machine learning interatomic potential (MLIP), machine learned charge density (MLCD), and/or machine learning property predictor (MLProp), each of which are detailed hereinbelow.

In another embodiment, at least one of a MLIP, MLCD, and MLProp may each compute analytical models based on training data from the DFT subsystem. Further, once the training data has been analyzed and/or adjusted, the process may again go through additional machine learning simulation. Additionally, the process may then verify whether or not the output atomic structure sample is below or above the predetermined threshold, wherein the system may double check the atomic structure from the DFT subsystem. It is recognized that the use of more than one machine learning model may increase the accuracy of what is generated. For example, a machine learning system that only uses MLIP for generating new material may not take into consideration charge density and/or other properties associated with the atomic structure. As such, each machine learning model may provide a different view and/or context on the atomic structure of the material generated. Further, the inclusion of multiple machine learning models may increase the accuracy of the resulting prediction for novel materials.

In one embodiment, the machine learning process may train on 80% of known data, perform verification with the remaining 20%, and determine whether the output data may actually still be non-conforming when compared with the threshold value(s). As a result, non-conforming samples may be passed back to DFT where additional data may be added to one of more of the machine learning models, and repeat the machine learning training process until the models perform within threshold guidelines. Of course, the machine learning process may segment the data into any percentage of training data and verification data as needed.

In one embodiment, the at least two machine learning systems may be configured such that the prediction can be outputted in fewer processing cycles compared to conventional computing systems. Thus, a machine learning model may not necessarily produce more accurate results than DFT, but machine learning may produce the same accuracy levels with a significant reduction in the amount of computational time invested. Additionally, as discussed, the inclusion of multiple machine learning models may increase the likelihood of accuracy with actual DFT simulation verification.

In one embodiment, the machine learning process may require a higher, more stringent threshold by which the sample atomic structure(s) may be compared to determine success, wherein the training process will likely take a longer time to complete, but yield greater volumes of usable data and mor accurate results. In another embodiment, the machine learning process may focus on fewer data points, wherein the learning process may be accelerated while simultaneously producing decreasingly accurate results. In still another embodiment, the MLIP, MLCD, and MLProp models may all receive the same atomic structure, but work to predict different threshold targets. Additionally, different atomic structures may be passed to DFT at different stages in the machine learning process.

### MLIP Model

With respect to MLIP, in one embodiment, the role of a machine learning interatomic potential (MLIP) model may be to predict a force vector on every atom within a structure that is based on specific configurations of atoms in relation to the total energy of that system. In practice, the MLIP model may take a snapshot of an atomic structure, including estimates of the forces applied to the atoms therein. Next, the MLIP model may allow a tiny movement in the direction of the forces that are pushing, followed by noting and recording the atoms' change(s) in location. For example, within a system at a hot temperature, the atoms within that structure typically move around a lot. By recording that movement information, the MLIP subprocess may construct a model to determine whether the material in question is stable. If the material is not stable, and the process progresses forward many steps in time, it may be expected that the structure will fly apart, and the determination may be made that such a structure will not hold together as a material. Alternatively, the MLIP model may determine that specific configurations of atoms are stable and may likely hold together well, and that you can expect that, once development progresses to the experimental stage, the stable material in question is something capable of being created in a lab and should remain stable.

Further, machine learning interatomic-potential (MLIP) be used as a machine learning model to predict the interactions between atoms in materials. MLIP models may train on a dataset of known atomic configurations (such as an initial dataset from DFT) and their associated energies and forces. Once trained, MLIP may rapidly predict the behavior of atomic systems under different conditions, making it more efficient than other conventional methods. For example, MLIP may enable researchers to simulate larger systems or longer timescales than previously attainable, which, as a result, may allow for a more in-depth exploration of material behaviors, properties, and transformations, and may lead to accelerated discovery and understanding of novel materials and phenomena. As such, MLIP may be used to model atomic level interactions.

### MLCD Model

With respect to MLCD, in one embodiment, the new dataset for the desired material may include electronic properties. As such, machine learned charge density (MLCD) may be used as a machine learning model to predict the density of electrons directly. In one embodiment, MLCD may be used to determine out a location and/or density of an electron field throughout a particular atomic structure for a set of atoms. In one embodiment, the MLCD model may apply machine learning to the prediction process in order to accelerate that process.

Further, machine learned charge density (MLCD) may be used as a machine learning model to predict the charge density given a specific material configuration, where the charge density may be a discretized scalar density field that spans the volume represented in the atomic structure.

In another embodiment, a collection of grid points may be plotted among the atoms in a sample atomic structure. For example, a charge density plot may identify a single scalar value for as many as 10 million grid points associated with 200 known atoms in three-dimensional space. Additionally, a grid point survey may reveal a collection of certain atoms in a certain concentration indicating a density of electrons at a specific point. As such, a collective and comprehensive view of charge density, as well as interactions may be modeled using MLCD.

### MLProp Model

With respect to MLProp, in one embodiment, the MLProp may encompass multiple different submodels, wherein the submodel(s) may each have an input (such as a structure) and an output (such as a single scalar value for a property that the material may have). In the instance of a wire comprised of pure copper, for example, a prediction regarding the electrical conductivity of the copper wire may be a single value output for that given structure.

Further, machine learning property predictor (MLProp) may be used as a machine learning model to predict a scalar value property associated with the entire material configuration. For example, MLProp properties may include, but not be limited to, band gap, electrical conductivity, formation energy, thermal conductivity, material strength, elastic modulus, hardness, density, thermal expansion, dielectric constant, magnetic properties, optical properties, corrosion resistance, chemical reactivity, wear resistance, sound absorption, tensile strength, fatigue life, etc. As such, properties of a material may be modeled using MLProp.

In one embodiment, the at least two machine learning models (e.g. MLIP, MLCD, MLProp, etc.) may be used in parallel. Alternatively, in another embodiment, the at least two machine learning models may be used in serial.

### Dataset Generation

In use, a dataset generation phase (controlled by a dataset prediction manager) may be implemented using a prediction subsystem that employs the previously-trained material models (e.g., the trained machine learning models such as MLIP, MLCD, MLProp, etc.) along with an input dataset comprising atomic structures and quantum data for dataset inference, prediction, and output.

In one embodiment, the GAD application may output, using the at least one computing device, a generative model. In one embodiment, the generative model may be designed to generate new data samples that resemble a given training dataset (such as from DFT). These generative models may learn the underlying patterns and structures in the training data and use such information to then generate new, similar instances. As such, the generative model may include new data samples based on DFT training data and one or more machine learning models (such as MLIP, MLCD, and/or MLProp).

In one embodiment, execution of the sequences of instructions to practice certain embodiments of the disclosure may be performed by a single instance of computer. According to certain embodiments of the disclosure, however, two or more instances of a computer system may be coupled by a communications link (e.g., LAN, public switched telephone network, or wireless network) and may perform the sequence of instructions required to practice embodiments of the disclosure using two or more instances of components of the one or more computer systems.

In one embodiment, the GAD application may determine, using the at least two machine learning models associated with the at least one computing device, an accuracy of the generative model. For example, the accuracy may be determined by comparing the generative model to the DFT training data, comparing the generative model to a likelihood of success to one or more of MLIP, MLCD, and/or MLProp models, etc.

In a related embodiment, the generative model may be outputted within an active learning computing environment. In this instance, the GAD application model may comprise an instance of an error computing module. For example, the error computing module may function as part of an active learning framework where model errors and uncertainty estimates are computed at the end of a training phase and/or a training epoch/regime, and a decision is made as to whether or not a newly evaluated structure must be recomputed with DFT. In this manner, after training data is provided to the GAD application, and new data is generated using the machine learning models, the GAD application may then determine whether the outputted data is sufficiently below a maximum threshold (of error) to then be evaluated with DFT. For example, if an output has a low chance of success (such as based on a charge density profile from MLCD), such an output would not be of sufficient quality to then be tested using DFT. In this manner, only data that has already been scrutinized and found of sufficient quality is then passed on back to DFT for further evaluation.

In one embodiment, the prediction may be output based on the generative model. Additionally, as discussed, the prediction, in one embodiment, may be outputted when the generative model meets the predetermined threshold. A desired threshold may be chosen by simulation/experimentation within a reasonable range. It is to be appreciated that the desired threshold may be determined as needed by the user, by the machine learning system, and/or by the context (a minimally used material may have a higher threshold of error than one that will be used in public transport, etc.). If a computed uncertainty and/or model error for a candidate material configuration (periodic or non-periodic atomic or molecular structures) is above the chosen threshold (i.e. maximum error level), then the candidate atomic structure may be passed back to the DFT subsystem to recompute accurate energy values, interatomic forces, charge density, and one or more property values via the machine learning models (e.g. MLIP, MLCD, MLProp, etc.). Further, the computed energy (e.g., global energy value), interatomic forces, charge density, and the one or more property values may then also be used to further train the MLIP, MLCD, and MLProp machine learning models.

Further, if the model error for a candidate material configuration (e.g., atomic structure) is below the chosen threshold, then the model is promoted to be included into an output dataset. In a related embodiment, the prediction may include iteratively reassessing the dataset for the desired material with the properties of the desired material until the properties of the desired material meet a predetermined threshold (i.e. be below a maximum error value).

In another embodiment, the prediction may be output using, at least in part, Bayesian Optimization (BO) to find, for example, an optimal set of parameters (including atomic structure) to satisfy a predetermined objective (such as compliant with the machine learning models). Based on the new material requirements, one or more available methods and/or tools (e.g., genetic algorithms, Monte Carlo methods, Bayesian optimization, etc.) may be used to generate inputs for the one or more atomic structures (either periodic atomic structures with associated period boundary conditions or non-periodic atomic structures such as molecules). Such inputs may function as appropriate kernels for creating desired structures as a part of an initial dataset.

Additionally, certain instances of an initial dataset may further comprise associated quantum data (for example, quantum states, Hamiltonian operators or functions, correlation functions, etc.) corresponding to the atomic structures and properties chosen and which typically may not be easily modeled using DFT techniques (and/or the machine learning models such as MLIP, MLCD, MLPRop, etc.). As such, in operation, the GAP application and system may work when trained on an initial dataset that is representative of a material class of interest, which includes a set of known materials (and/or atomic structures) and their associated properties, and the system may then learn and suggest newer configurations that may optimize a property or properties of interest.

In one embodiment, the prediction may be output in fewer computing cycles compared to a conventional computation of the one or more datasets. Once a predictive model has been established, a development cycle may begin by generating one or more structures using a second MLIP model process (or a second model process using MLCD and/or MLProp detailed hereinbelow). As such, the second MLIP model may inform as to whether structures are stable by predicting the forces on the atoms and may run simulations that evolve over time to demonstrate whether the predicted atomic structure remain stable or become unstable.

Second MLIP model processes may run simulations more quickly than otherwise traditional physical experimentation. In one embodiment, the second MLIP model process may generalize to one or more atomic structures that are deemed to be adequately similar to known training data. For example, given a hypothetical model where 10,000 atomic structures may be involved, a single material analysis may take as long as 30 minutes to complete. Employing the use of a second MLIP, however, may provide enough processing power to complete analysis for a single atomic structure in a fraction of a second, thus facilitating perhaps otherwise impossible workloads within a single MLIP model process.

In still another embodiment, predictions may be capable of being run on one or more proposed novel atomic structures that the DFT subsystem has not yet analyzed, itself.

In another functional embodiment, the second DFT subsystem may be employed to verify dataset(s) output for the one or more novel atomic structures. As such, the input to the second DFT subsystem may be the one or more atomic structures and the output data may include the properties that the one or more atomic structures possess, and both of those may be fed to subsequent downstream task(s).

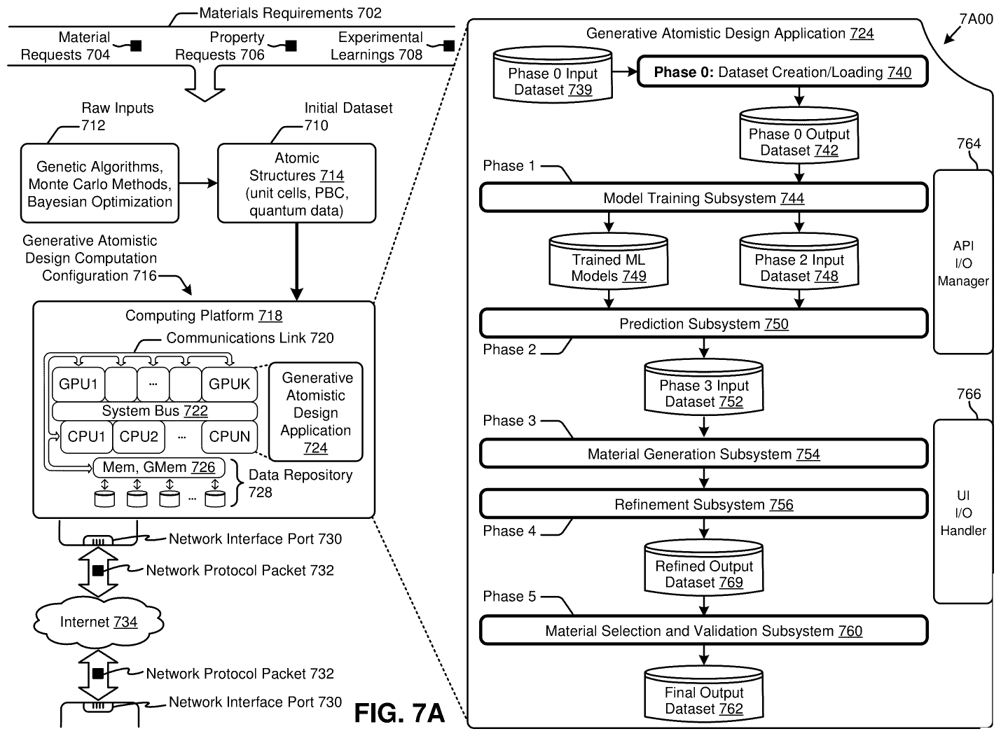

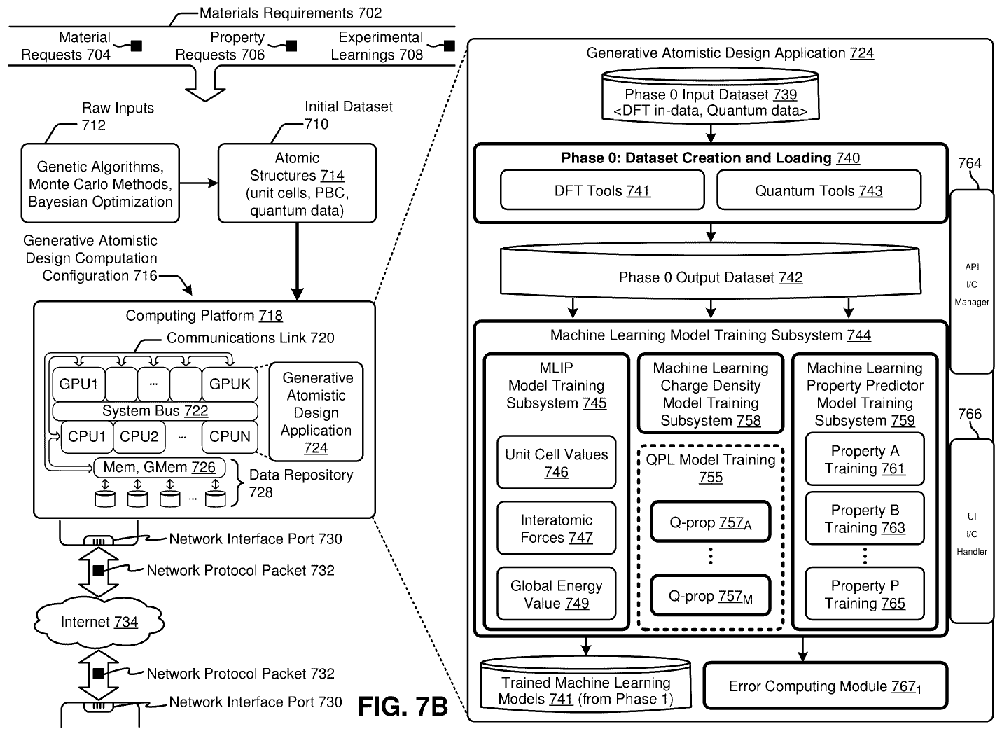

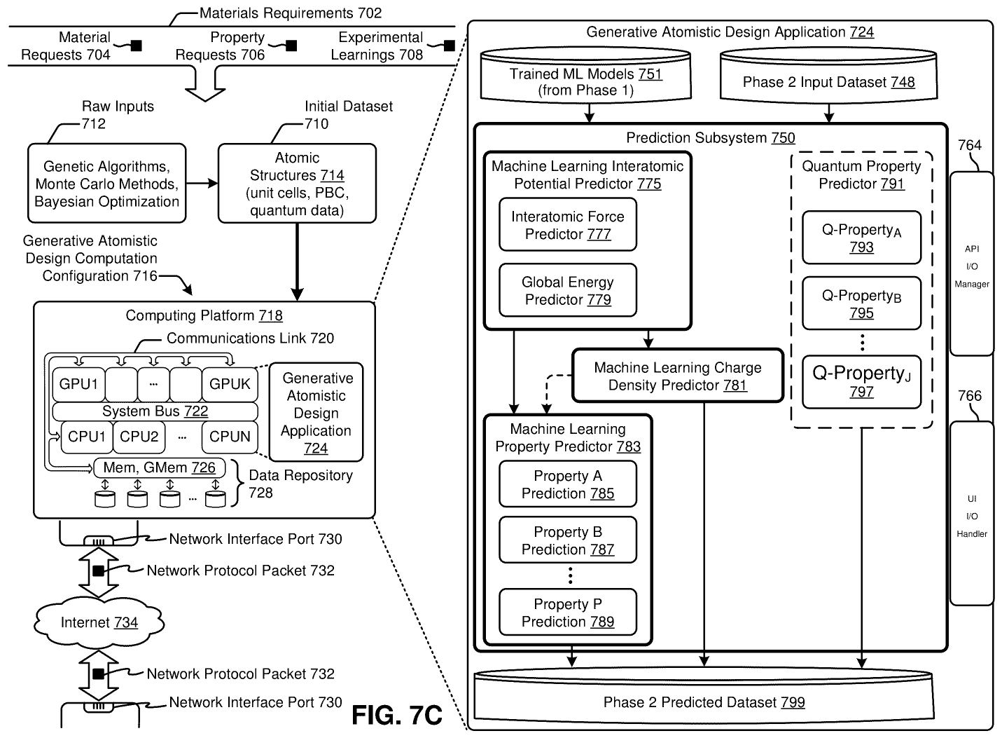

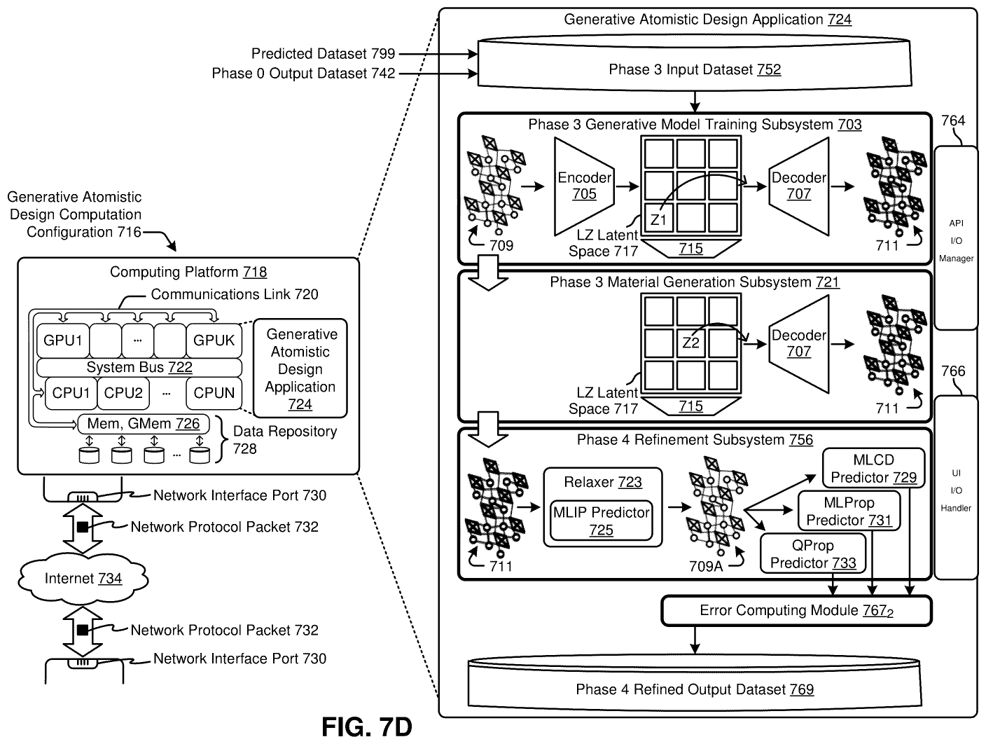

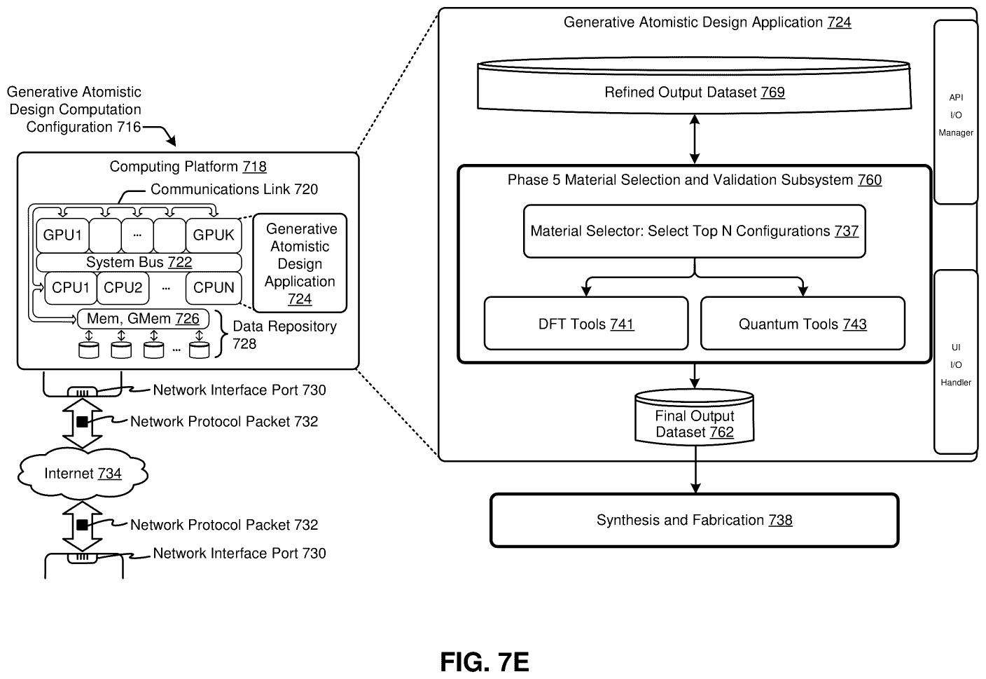

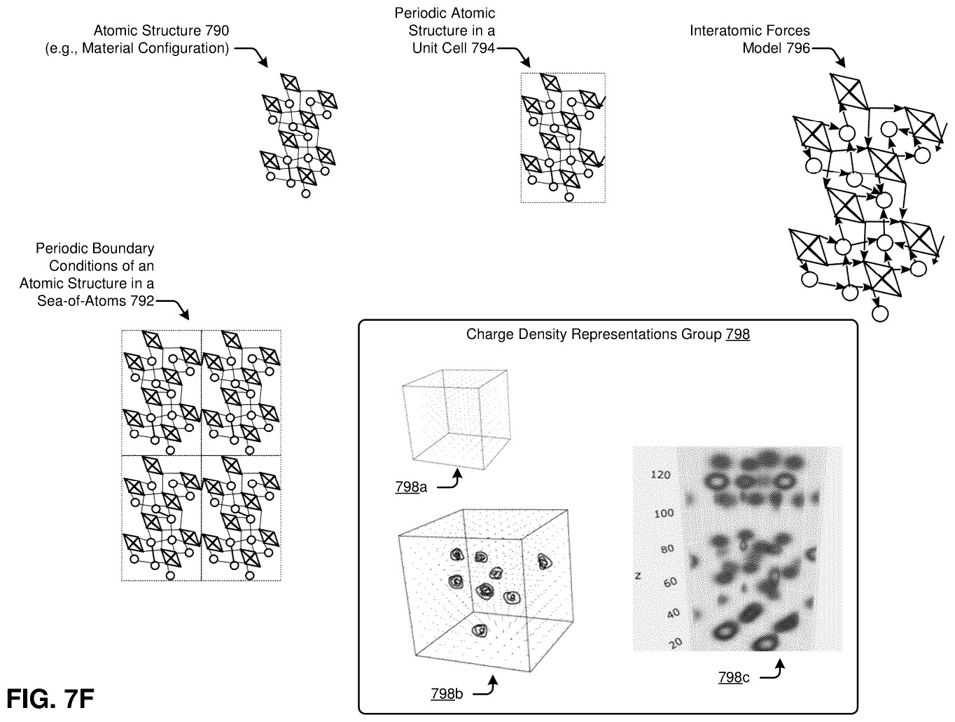

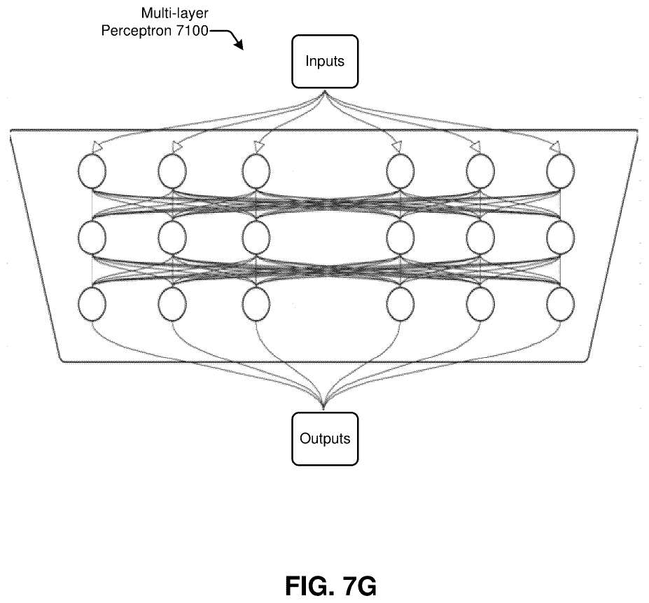

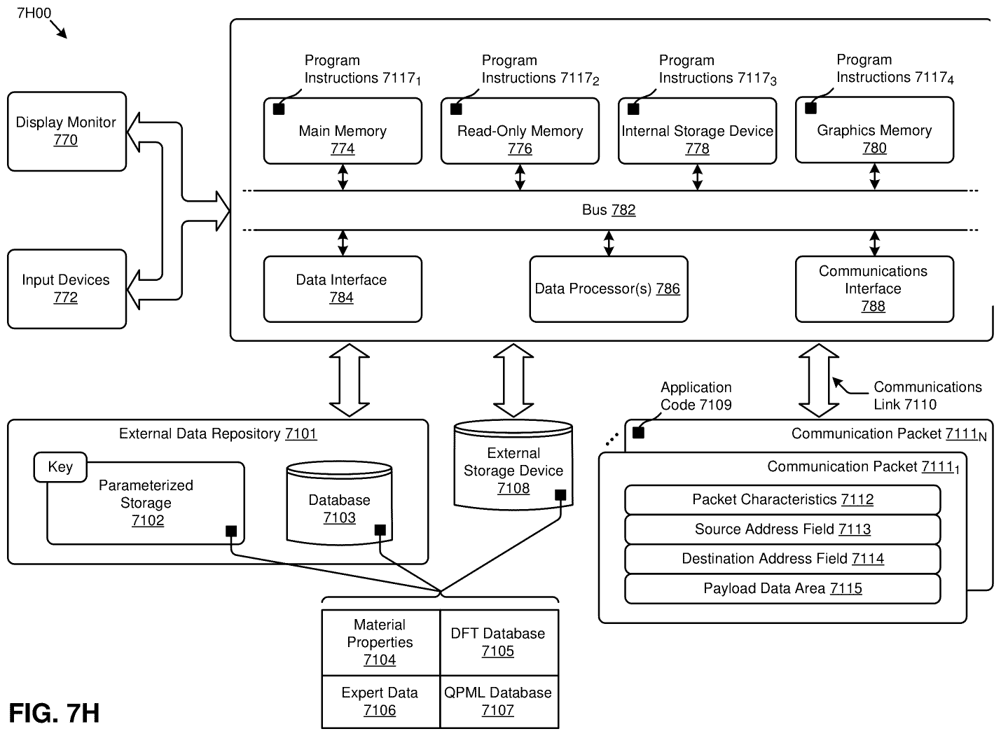

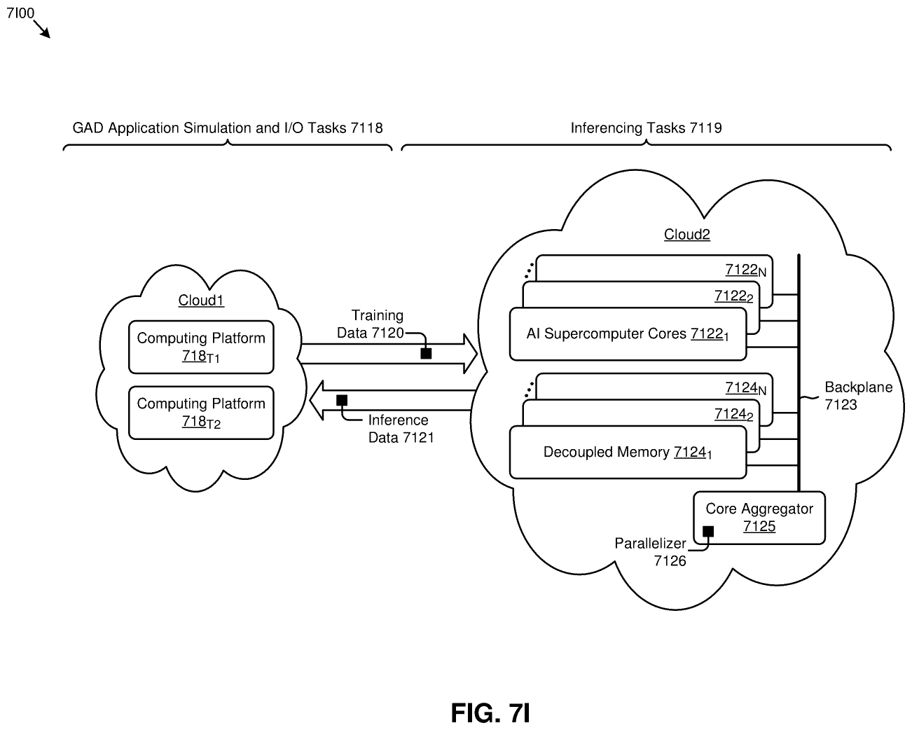

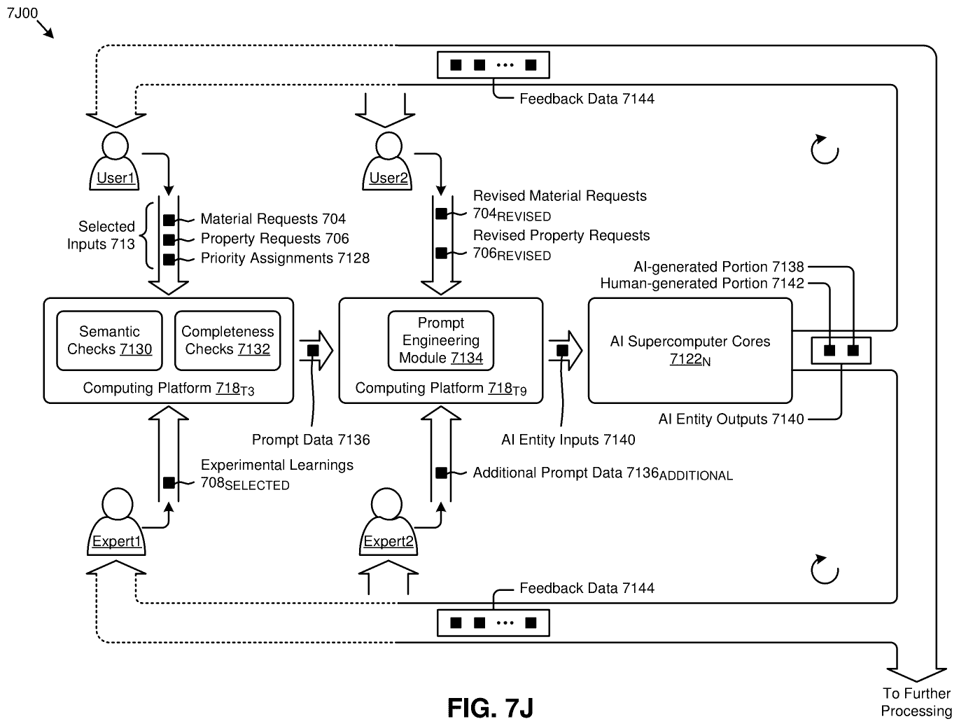

For example, a second MLCD may predict the charge density of the resulting one or more structures, and the second MLCD may then also predict the properties of the one or more structures, and again, those structures and associated properties those may be fed to subsequent downstream task(s). It should be noted that an overall model verification process, including second DFT, MLIP, MLCD, and MLProp models, may occur in a serial/linear mode, as well as in parallel. Further, it should be noted that, as used herein, a second model (or anything beyond an initial model) is simply a trained version of the initial (or prior) model. For example, as described in greater detail hereinbelow, the Trained ML Models (such as block 749 of FIG. 7A) may be used by the Prediction Subsystem 750 and/or the Refinement Subsystem 756. As such, prior models may represent a trained dataset which can then be used to predict and then generate new material.

In one embodiment, a second MLProp model may use the second MLCD output data to perform analytical functions on the one or more atomic structures that result from the second DFT subsystem processing. In another embodiment, a second MLProp model may focus on positioning and types of atoms, wherein another model type may analyze the positioning and types of atoms with regard to the atoms' charge density data output from the second MLCD model.

In this manner, the MLIP, MLCD, and/or MLProp models may be used repeatedly (in an active learning environment) to test and refine potential new candidates for material generation. As such, this process (of continual experimentation and testing) may be akin to a neural network. For example, a neural network may learn patterns within the second MLIP model. Additionally, such formulae may be part of the second DFT subsystem and the second MLIP neural network may inherently learn patterns based on known training data. In other words, a neural network may learn the relationship between one or more atomic types, their respective positioning, and resulting forces thereupon. Additionally, within the active learning environment in which this analysis may take place, each iterative analysis may increase in efficiency (as it may build upon the subsequent learned information and data).

In one embodiment, an additional QPML component may provide supplemental information to be added into the modeling process for the one or more atomic structures and their respective properties. For example, as there may be some aspects of material science that may not have been affected at modeling, an additional QPML model may augment the analysis of the known set of one or more atomic structures.

In any case, quantum data may be integrated within the predictive machine learning phase (in combination with MLIP, MLCD, and/or MLProp models) to assist with analyzing and predicting novel stable materials.

For example, a spin hybridization process, in which there may be specific types of atoms that are interacting with one another, may comprise electrons in specific orbitals with particular spins that correlate with one another and may not be modeled within a typical DFT subsystem. Such information may affect the downstream properties during material analysis. As such, the modeling and verification process may run through the second MLIP, MLCD, MLProp models, as well as include an additional QPML component to yield a prediction with higher of fidelity and/or accuracy.

In another tangible example, an additional QPML may be much more accurate in predicting excited states of atomic structures, where the systems may become energized and the electrons therein may jump to higher orbitals (as opposed to settled or relaxed material states), than might be achieved using just a DFT processing model.

In one embodiment, the additional QPML approach may be based on a Hamiltonian learning process, in which the quantum learning model may attempt to create an equation representing states of the one or more atomic structures and their respective evolution over time. Based on the new material requirements, one or more available methods and/or tools (e.g., genetic algorithms, Monte Carlo methods, Bayesian optimization, etc.) may be used to generate inputs for the one or more atomic structures (e.g. periodic atomic structures with associated period boundary conditions, non-periodic atomic structures such as molecules, etc.). Such inputs may function as appropriate kernels for creating desired structures as a part of an initial dataset. Certain instances of an initial dataset may further comprise associated quantum data (for example, quantum states, Hamiltonian operators or functions, correlation functions, etc.) corresponding to the atomic structures and properties chosen and which typically may not be easily modeled using DFT techniques. As such, quantum data may be included in the predictive machine learning process to increase the likelihood of success of the resulting novel material. Previously, it was discussed that a higher number of machine learning models (MLIP, MLCD, MLProp) may assist with increasing a higher accurate prediction. In like manner, inclusion of quantum data within the context of the machine learning analysis may also assist with a higher accurate prediction.

In one embodiment, a second MLProp prediction model may comprise only one property, or a single number for a single structure. In addition, it may be possible to perceive multiple different properties made available to a second MLProp prediction model. As such, the process may involve multiple different property models for specific properties. For example, one model that takes in the structure and predicts electrical conductivity and/or another model that takes in the structure and predicts heat capacity and other properties may be employed. As a result, a typical scenario may comprise multiple MLProp models that may result in different property values.

In one embodiment, threshold values against which candidate atomic structures may be measured (including but not limited to charge density, properties, among others) may take one or more aspects and/or properties into consideration when determining whether a threshold value has been met or satisfied. It should be noted that a threshold parameter may typically depend on the scale of the threshold value(s) themselves.

In another embodiment, when seeking to further refine the one or more atomic structures via DFT and the machine learning models, a second MLProp model may be chosen to retrain and incorporate analyses in order to generate candidate atomic structures that selectively optimize one or more specific properties that, for example, may maximize electrical conductivity or stability. Additionally, with this particular embodiment, confidence that the candidate atomic structures are stable may be achieved due to the fact that other known properties, such as charge density data, may be output with the candidate structure data which may be indicative of stability. In still another embodiment, it is to be appreciated that the second MLProp model analyses may be bypassed (or skipped), wherein the process may still generate candidate atomic structures that may be more likely to result in less accurate or stable structures.

In one embodiment, the second DFT subsystem may compute charge density via a second MLCD. In one embodiment, the generated model output may provide a full output including not only the position and location of atoms within the atomic structure(s), but also the charge density as well. Thus, when the modeling process passes candidate atomic structure datasets back through the second DFT subsystem, the dataset may function as a starting (or index) point for a particular property, such as charge density, which may alleviate what might typically require an entire computationally-expensive process to refine a prediction about charge density. As an example, when a given predicted charge density is introduced to the second DFT subsystem as part of a candidate atomic structure dataset, the resulting refining process may require just two additional optimization steps instead of scores or even hundreds.

### Materials Generation

With respect to generation of the materials, a materials generation phase may be implemented using a material generation subsystem that receives an input dataset comprising multiple atomic structures (or atomic structures with periodic boundary conditions (PBCs)) along with corresponding/associated charge densities and/or one or more predicted properties (i.e., property values) associated with each of the corresponding periodic atomic structures. The material generation subsystem may train a generative machine learning model to generate new material configurations comprising unit cells, and/or periodic unit cells, and/or other representations of atomic structures.

In one embodiment, the creating of the machine learning models may experiment via Bayesian Optimization (BO) to predict the desired material. For example, charge density may determine the property of a candidate atomic structure. As such, electrons' position(s) around the atoms in an atomic structure may determine which outside (external) forces interact with the structure and in what way(s) those forces may impact the structure (such as the manner in which light bounces off of a structure, or the manner in which electrons move through the structure). Thus, charge density may be considered a foundation of atomic structure properties. It is to be appreciated that charge density is but one example and other properties may be used as a basis for a foundation of atomic structure properties.

In one embodiment, the generation of one or more candidate atomic structures may comprise a compression-and-decompression phase. The training phase in question may collect and compile a list of values representing a candidate atomic structure, train a machine learning model to apply the compressed compilation from an original material construction to reconstruct a novel flavor of the original atomic structure, and measure the resulting novel structure with known threshold parameters for the composition. As such, within the machine learning model, a latent space, which may include possible combinations of compressed numbers, may result from a reconstruction/decompression. Further, any combinations from the latent space that are not previously known combinations in the machine learning model may become new training data for the model to be used in subsequent iterations of the verification process. In one embodiment, therefore, different vectors may be selected and novel candidate atomic structures may be realized. For example, a quasi-random list of 64 values with which to generate a novel candidate atomic structure may be selected that may be found to be a valid novel material and may take advantage of the associated predictive machine learning model(s) that have been trained, using the compression and decompression process, to deliver an already trusted dataset for novel atomic structures.

It must be emphasized that the way in which the initial vector from which the generative model may originate is selected may be determinative. Additionally, the benefit that may be derived from the compression and decompression processes may be based on a latent space organized around a specific property being trained. Further, a machine learning training model may focus on a specific region of a latent space known to host a notable concentration of vectors, wherein the specific property sought to be trained may appear at a relatively high frequency, thus potentially making that latent space a desirable location from which to procure one or more additional material samples for generating candidate atomic structures.

In one embodiment, one or more vector databases may be compiled from a generative modeling process. Additionally, a similarity between vectors in the vector databases may be determined.

### Materials Refinement

With respect to materials refinement, in use, a refinement phase may be used where material configurations (atomic structures, unit cells, etc.) previously generated by a generative machine learning model may be received by a refinement subsystem to refine the one or more generated structures. Additionally, the refinement subsystem may analyze charge density and property values of the generated structure. In one embodiment, the refinement subsystem may be used to relax the atomic structures and make them stable by adjusting the locations of atoms so as to reduce interatomic forces inside the atomic structures. The foregoing may create refined datasets with refined atomic structure systems.

In one embodiment, the second MLIP may inform as to which forces are currently affecting atoms in a candidate atomic structure. Thus, those forces may be comparatively high, indicating that the structure may be unstable (i.e. error notification). Alternatively, however, the forces acting upon the atoms in the structure may, predictably, not be very high, which may allow the refinement subsystem allow the structure to relax. As such, the position of the atoms within the resulting structure may shift to a greater or lesser degree such that the atomic structure may become more stable (through an active learning cycle, in one embodiment, of iteratively processing and reviewing the atomic structure). In practice, the iterative nature of recording tiny shifts in the atomic structure during this phase may entail recording the state of the structure, taking a small step in time, noting changes in the structure and calculating forces in effect, and repeating the process until the maximum force(s) applied to the structure fall below a known error threshold, at which time the relaxing process may terminate.

Within the context of the present description, structure relaxation may refer to the process by which an atomic structure is modified. For example, structure relaxation may include adjusting an atomic or molecular configuration to minimize its energy, resulting in a more stable (or equilibrium) state. In practice, when atoms or molecules are initially placed in a simulated environment, they may not be in their most energetically favorable positions. In response to this condition, structure relaxation may allow these atoms or molecules to “settle” into their lowest energy configurations, which may often correspond to the state the atoms or molecules might naturally assume, themselves.

In addition, structure relaxation may become particularly efficient in the context of MLIP. For example, traditional methods might require intensive computational resources to relax complex atomic structures. In the case of MLIP, however, the process may be expedited significantly by leveraging machine learning to predict the manner in which atoms may move to achieve their minimum energy state based on patterns and data on which it was previously trained. As such, MLIP model may allow for faster, and often more accurate, relaxation processes, which may aid in the efficient exploration and understanding of material behaviors and properties. As such, the machine learning models may be used to determine atomic structures with greater stability.

In another embodiment, the second MLIP model may receive output comprising one or more candidate atomic structures from the generative model, wherein the second MLIP modeling process may look at every single atom and may identify previously unseen and/or unrecorded atoms in close proximity to other atoms, and may predict force vector(s) on that atom. As such, the second MLIP model may highlight any affecting forces and their respective magnitude(s), may establish one or more predictions about the atoms in the structure, and may then allow all of the atoms to move in response to the forces at work upon the structure, all the while recording the movement and interrelationships over time.

In still another embodiment, the second MLIP modeling process may be used to reduce forces by employing a heuristic and determining that one or more forces are too high (the atomic structure is in an “unsettled” state). As such, the second MLIP modeling may then affect a tiny movement of one or more atoms in an effort to reduce the force(s) being applied. Thus, the second MLIP may be thought of as being used to perform molecular dynamics (that is, allowing the atoms to move or shift based on the forces being applied).

In one embodiment, the process of refining the collection candidate atomic structures may involve additional compression-and-decompression exercises based on the generative model(s), wherein the process may relax the structure(s) within the second MLIP model, recompute charge density, and reassess the state of any additional QPML properties in order to output further refined candidate atomic structures based on the initial generative ML model. It is to be recognized that reassessing the state may include any additional properties including, but not limited to MLProp, QPML, etc.

As such, a semi-supervised learning environment may be used to create new data sets with atomic structures and properties based on machine learning models (MLIP, MLCD, MLProp). Further, as discussed, the new data sets may be further scrutinized (and/or supplemented) with quantum data and/or a quantum machine learning model. Lastly, having an active learning cycle based on the generative model assist with further refining the material prediction output such that the material prediction output is stable.

Further, this arrangement of training a machine learning system with an initial dataset, and then using the trained data as the basis for then generating new materials helps to create a process whereby the machine learning system has a solid foundation to create additional datasets based on the initial dataset, and then the additional datasets create a data-rich environment where novel materials can be generated and refined in a highly accurate manner.

As shown, the method 1B00 may output, using the at least one computing device, a prediction comprising the desired material. See operation 1B06.

In various embodiments, a material selection and validation phase may be implemented using a material selection and validation subsystem, which may take in a refined dataset (e.g., a refined output dataset) and select one or more candidate materials, and their corresponding/associated data, which may be stored in a final output dataset. Additionally, the material properties and charge densities of the candidate materials may be accurately computed in this phase using the DFT tools and models. In addition to the foregoing candidate materials as computed in this material selection and validation phase, other properties and/or aspects of a material and its simulation model counterparts may be stored in final output dataset for eventual synthesis and production.

In one embodiment, the GAD application may synthesize the desired material(s) based on the prediction. Outputting a prioritized collection of candidate atomic structures may involve further verification of the top <n> candidates within the final output set, and may entail then selecting a subset of candidates that may be chosen for actual physical synthesis and experimentation. Additionally, the generation process may further comprise synthesizing the desired material based on prediction(s).

Within the context of the present description, synthesis may refer to the process of creating new materials. In one embodiment, synthesis may be achieved by inducing chemical reactions between different substances. Synthesis may involve carefully selecting and combining precursors, as well as applying external factors (such as heat, pressure, and/or light), to instigate desired reactions, where the goal may be the production of novel compounds or materials with specific properties or structures. For example, a researcher may synthesize a new ceramic material designed to withstand extreme temperatures, or may create a new polymer with unique mechanical properties. Thus, synthesis may be a foundation of materials discovery, enabling the creation of new materials that might not exist naturally. It is to be appreciated that synthesis (the process of actually creating the material) builds upon the machine learning system output, which includes the actual atomic structure.

FIG. 1C illustrates a system 1C00 for reiterative model training with quantum capabilities, in accordance with one embodiment. As an option, the system 1C00 may be implemented in the context of any one or more of the embodiments set forth in any previous and/or subsequent figure(s) and/or description thereof. Of course, however, the system 1C00 may be implemented in the context of any desired environment. Further, the aforementioned definitions may equally apply to the description below.

As shown, the system 1C00 may receive one or more datasets corresponding to desired material classes and properties associated therewith. See operation 1C02. In practice, artificial intelligence and/or machine learning systems may be leveraged to make accurate predictions regarding the properties that a specific material type may have, and significantly reduce the amount of time required in a physical lab to realize accurate simulations and accurate results. An initial dataset creation/loading phase may be implemented using dataset creation/loading subsystem, which receives initial dataset that may be taken in as initial phase input dataset, in any appropriate format, and added to existing instances of input datasets. The updated instances of input datasets may then be used to load and/or create further datasets using one or more DFT-based tools for use in subsequent phases.

In one embodiment, the one or more datasets (including the desired material and properties of the desired material) may be received and analyzed using one or more Density Functional Theory (DFT) models. As such, DFT may be used as a computational approach for creating simulations of novel materials that fulfill specific needs. For example, wherein a base level of testing may employ physical experimentation, an alternative method for determining atomic structure stability may employ computational methods within a DFT paradigm to help facilitate that process. As such, the initial training dataset may be received and/or analyzed using DFT.

Additionally, the GAD application may train active learning models by reiteratively applying a combination of two or more machine learning subsystems, including, but not limited to MLIP, MLCD, MLProp, and QPML subsystems. See operation 1C04.

In one embodiment, at least two of a MLIP, MLCD, and MLProp may each compute analytical machine learning models based on training data from the DFT subsystem (per operation 1C02). Further, once the training data has been analyzed and/or adjusted, the system 1C00 may again go through additional machine learning simulation. Additionally, the output atomic structure sample may then be verified whether or not the output atomic structure sample is below or above the predetermined threshold, wherein the system may double check the atomic structure from the DFT subsystem.

In another embodiment, the machine learning process (via operation 1C04) may require a higher, more stringent threshold by which the sample atomic structure(s) may be compared to determine success. Additionally, in one embodiment, the training process may take a longer time to complete based on the higher threshold, but yield greater volumes of usable data and more accurate results. In another embodiment, the machine learning process may focus on fewer data points, and the learning process may be accelerated while simultaneously producing decreasingly accurate results. In still another embodiment, the MLIP, MLCD, and MLProp models may all receive the same atomic structure, but work to predict different threshold targets. Additionally, different atomic structures may be passed to DFT at different stages in the machine learning process.

In one embodiment, an additional QPML component may provide supplemental information to be added into the modeling process for the one or more atomic structures and their respective properties. For example, as there may be some aspects of material science that may not have been affected at modeling, an additional QPML model may augment the analysis of the known set of one or more atomic structures.

Further, the GAD application of the system 1C00 may evaluate the resulting uncertainty estimates and error calculations to determine whether additional training may be necessary to achieve optimal active learning model creation (see decision point 1C06) and, ultimately, output a trained model for generative purposes as in 1C08 when the error is below a maximum error threshold. If the uncertainty estimates and error calculations exceeds the maximum error threshold, the system 1C02 may loop the dataset back to the machine learning subsystems (including but not limited to MLIP, MLCD, MLProp, and QPML subsystems) for further analysis and refinement.

In one embodiment, the prediction of the desired material may be output based on the generative model, and in one instance, may be outputted when the generative model satisfies a predetermined error threshold. A desired threshold may be chosen by simulation/experimentation within a reasonable range, and if a computed uncertainty and/or model error for a candidate material configuration (periodic or non-periodic atomic or molecular structures) is above the chosen error threshold, then the candidate atomic structure may be passed back to the DFT subsystem to recompute accurate energy values, interatomic forces, charge density, and one or more property values. The computed energy (e.g., global energy value), interatomic forces, charge density, and the one or more property values may then also be used to further train the MLIP, MLCD, and MLProp machine learning models (as well potentially the QPML). Thus, if the model error for a candidate material configuration (e.g., atomic structure) is below the chosen error threshold, then the model may be promoted to be included into the output dataset. In a related embodiment, the prediction may include iteratively reassessing the dataset for the desired material with the properties of the desired material until the properties of the desired material meet a predetermined threshold.

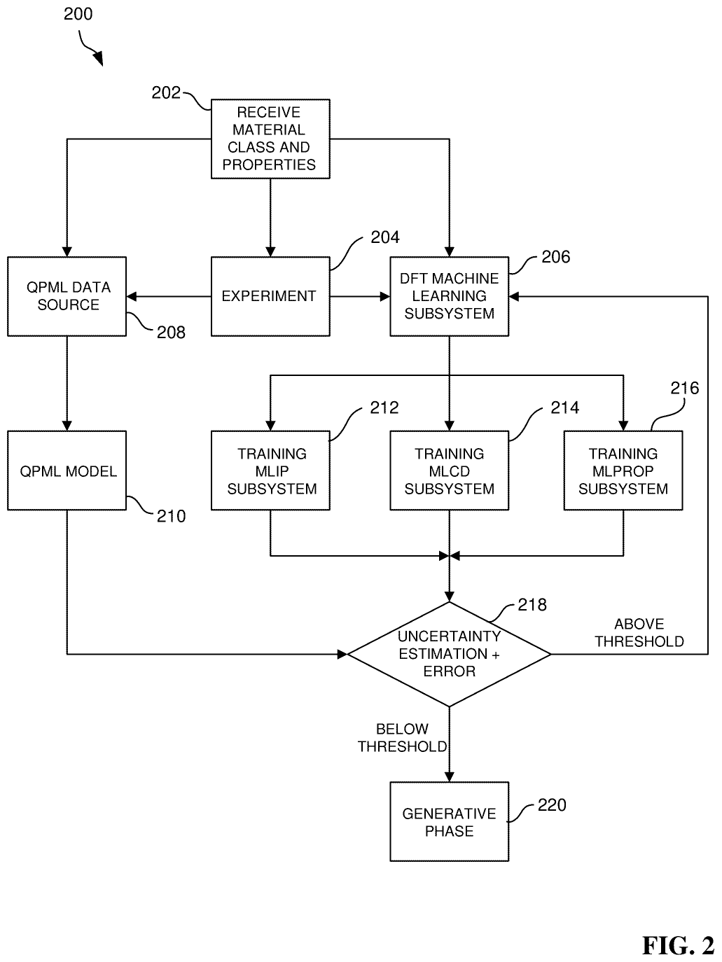

FIG. 2 illustrates a system 200 for outputting one or more atomic structure datasets trained via classical and/or quantum machine learning, in accordance with one embodiment. As an option, the system 200 may be implemented in the context of any one or more of the embodiments set forth in any previous and/or subsequent figure(s) and/or description thereof. Of course, however, the system 200 may be implemented in the context of any desired environment. Further, the aforementioned definitions may equally apply to the description below.

As shown, a generative atomistic design (GAD) application system may receive one or more material classes and associated desired properties. Such material classes and associated desired properties may define an initial dataset from which machine learning subsystems may further train a predictive atomic structure model. See operation 202. In practice, the dataset(s) received in operation 202 may be used to conduct physical experimentation 204, used as the source of density functional theory (DFT)-driven machine learning training subsystem 206, and/or supplemented via additional quantum machine learning (QPML) source data 208.

In one embodiment, physical experimentation 204 may further comprise experimental learnings in the form of measurement data (e.g., tables, files, records, etc.) such as measurements deriving from surface differential reflectivity (SDR), diffuse reflectance spectroscopy (DRS), diffraction data, interference waveforms, etc. Additionally, it is to be appreciated that the experimental data may not be limited to the properties listed in this disclosure and may, in the future, include newer properties not yet discovered or conceived of as of the filing date of this disclosure. Additionally, the intended area of exploration (via the physical experimentation 204) may inform which related density functional theory (DFT) computations (including any molecular dynamics simulations) or other quantum mechanical computations using techniques such as quantum many-body perturbation models (e.g., random phase approximation (RPA), GW approximation, etc.), density matrix renormalization group, dynamical mean field theory, variational quantum eigensolver (VQE), variational quantum thermalizer (VQT), etc.) may be performed to generate the initial training dataset.

In another embodiment, the DFT machine learning subsystem 206 may analyze and align the raw atomic structure dataset(s) to be used in subsequent GAD application training and generative operations, including a predictive material model training subsystem, prediction subsystem, material generation subsystem, refinement subsystem, and/or validation subsystem, as and when required. It is to be appreciated that the DFT machine learning subsystem 206 may use density functional theory to compute datasets that comprise electronic structures and properties of atomic configurations (e.g., atomic structures, charge densities, global energy, interatomic forces, global properties, etc.).

In yet another embodiment, additional QPML source data 208 may optionally enhance the initial atomic structure dataset(s) (from operation 202) via QPML model 210, which may be used to further compare predicted quantum property values from quantum property learning model 210 with generated quantum property values that result from DFT subsystem machine learning.

In various embodiments, DFT machine learning subsystem 206 may facilitate more focused machine learning via machine learning subsystems: machine learning interatomic potential (MLIP) model training subsystem 212, machine learning charge density (MLCD) model training subsystem 214, and machine learning property predictor (MLProp) model training subsystem 216.

In one embodiment, the MLIP model training subsystem 212, controlled by a training manager, may further comprise an atomic structure model training capability, which may receive as input one or more atomic structures, unit cells, PBCs from the atomic structure dataset(s), which in turn may be used to train one or more periodic atomic structure models inside the MLIP model. In related embodiments, a portion of the machine learning MLIP model may be trained using one or more unit cells and/or periodic atomistic structures as received in an input atomic structure dataset.

In another embodiment, MLCD model training subsystem 214 may receive at least one set of charge density values used to train the machine learning charge density model (MLCD model) with the at least one set of charge density values for a specific material configuration received as input from DFT machine learning subsystem 206. The set of charge density values received may include a discretized scalar density field model that spans a volume representing an atomic structure, where the charge density values are scaler values distributed throughout a unit cell.

In still another embodiment, MLProp model training subsystem 216 receives at least one material property model and trains an internal predictive model on the specific material property/properties selected. The machine learning property model may be used to predict a scalar value of a global property associated with the entire material configuration (including the atomic structure) associated with it. Additionally, the global property examples may include, but not be limited to, band gap, electrical conductivity, formation energy, etc.

It is to be appreciated that the DFT machine learning subsystem 206 may use any or all of the MLIP subsystem 212, MLCD subsystem 214, and/or MLProp subsystem 216. Additionally, the DFT machine learning subsystem 206 may use the MLIP subsystem 212, MLCD subsystem 214, and/or MLProp subsystem 216 in a parallel or serial fashion. For example, in one embodiment, the initial dataset may be passed in parallel to all of the machine learning subsystems (the MLIP subsystem 212, MLCD subsystem 214, and/or MLProp subsystem 216). In another embodiment, the initial dataset may be provided sequentially first to the MLIP subsystem 212, which in turn may pass it along to MLCD subsystem 214, which in turn may pass it along to MLProp subsystem 216.

In an embodiment, after machine learning modeling in MLIP model training subsystem 212, MLCD model training subsystem 214, and MLProp model training subsystem 216, one or more trained models may be passed to an error computing module 218, wherein model errors and uncertainty estimates may be computed and a decision made as to whether the newly evaluated atomic structure satisfies (i.e. is below) an error maximum threshold. As such, if the errors and uncertainties exceed a selected threshold, the atomic structure may be passed back to the DFT machine learning subsystem 206, where the predictive machine learning models (e.g., models in the form of one or more atomic structures, models in the form of periodic boundary conditions, etc.) may be used to re-evaluate the atomic structure.

In addition, it should be noted that this feedback mechanism may be used to enhance or confirm any one or more of corresponding/associated global energy values, corresponding/associated interatomic force models, corresponding/associated charge density values, and/or corresponding global property values. In another embodiment, criteria for training convergence may be set even when, after the learning rate is sufficiently low, no further improvement in the error may be observed. As such, this feedback mechanism may be continued until the computed error, including uncertainty estimates, is below the selected threshold and the newly evaluated atomic structure may be passed to the system 300 (described hereinbelow) for outputting trained and refined generative machine learning models for candidate atomic structures.

In one embodiment, various sets of machine learning material models of atomic structures, associated periodic boundary conditions, associated global energy predictive models, associated interatomic force models, associated charge density values, and one or more associated global property value predictive models may be output in operation 220. These various sets of machine learning material models may then be used in other phases to perform various tasks/steps including inference/prediction, relaxation, estimation, refinement, and/or other tasks/steps not listed here.

It is to be appreciated that the system 200 may be intended primarily to train a GAD system. Once the GAD system is sufficiently trained (with a refined dataset that has passed through the machine learning model subsystems), the GAD system may then be used to generate new materials (via the system 300).

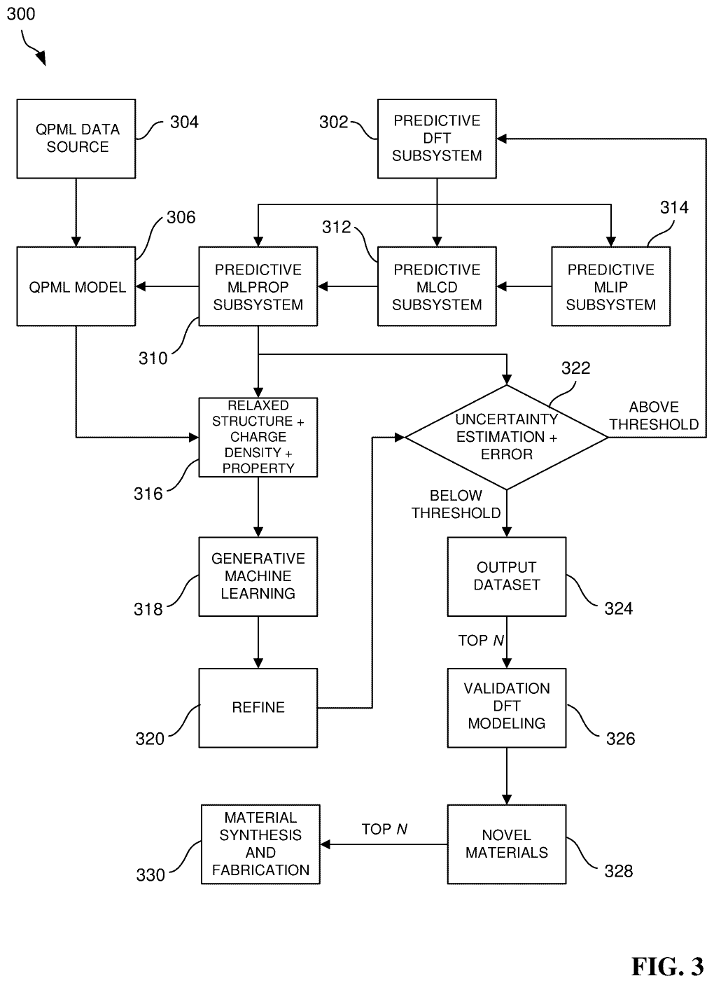

FIG. 3 illustrates a system 300 for outputting trained and refined generative machine learning models for candidate atomic structures, in accordance with one embodiment. As an option, the system 300 may be implemented in the context of any one or more of the embodiments set forth in any previous and/or subsequent figure(s) and/or description thereof. Of course, however, the system 300 may be implemented in the context of any desired environment. Further, the aforementioned definitions may equally apply to the description below.

As shown, a generative atomistic design (GAD) application system may create one or more atomic structure generative models based on the machine learning model output from operation 220. In some embodiments, the GAD application may undertake a dataset generation phase that is implemented using a prediction subsystem. As such, in the dataset generation phase, predictive machine learning may be used to predict and/or infer datasets for generative machine learning that follows. In practice, the machine learning model output from operation 220 may be used as the source data for DFT predictive subsystem 302 and associated MLIP predictive modeling subsystem 310, MLCD predictive modeling subsystem 312, and MLProp predictive modeling subsystem 314.

In one embodiment, DFT prediction subsystem 302 may comprise MLIP predictive models, MLCD predictive models, MLProp predictive models, and/or any other predictive models included in trained predictive models. In one embodiment, the DFT prediction subsystem 302 may include a MLIP prediction. Further, the MLIP predictive model may include the capability to predict interatomic forces, as well as the capability to predict global energy values, of candidate atomic structures. In related embodiments, the predictive models may be based on various neural network models such as graph neural networks, convolutional neural networks, transformers, tensor field networks, multi-layer perceptrons, etc. In addition, the output of the DFT predictive modeling subsystem 302 may be stored in memory as a data structure, which may be forwarded to other subsystems, subroutines, functions, or capabilities in various phases of the GAD application.

In some embodiments, QPML predictive model 306, which may draw source input from quantum properties data 304, may also be used to predict quantum property values and may be further used as a supplement to, and/or in combination with, the output of the DFT predictive modeling subsystem 302 to provide higher fidelity global property values where and when feasible for predictive modeling output via predictive error computing module 322. Further, an output from the MLProp predictive modeling subsystem 310 may also serve as an input to the QPML Model 306 such that atomic structure data may be further scrutinized and supplemented with quantum data.

In one embodiment, the GAD application system may conduct uncertainty estimation and error calculation upon the predictive modeling output via predictive error computing module 322, wherein model errors and uncertainty estimates may be computed with regard to the predictive model and a decision made as to whether further adjustment to the atomic structure are need, in which case, the system 300 returns back to the DFT predictive modeling subsystem 302. As such, if the errors and uncertainties exceed a predetermined and selected error threshold, the atomic structure may be passed back to the DFT predictive modeling subsystem 302. In one embodiment, this feedback mechanism may be used to enhance the predictive machine learning models (e.g., models in the form of one or more atomic structures, models in the form of periodic boundary conditions, etc.). Again, it should be noted that this feedback mechanism may be used to enhance or confirm any one or more of corresponding/associated global energy values, corresponding/associated interatomic force models, corresponding/associated charge density values, and/or corresponding global property values.

In an alternative embodiment, the GAD application system may further refine the one or more resulting predictive models created via DFT prediction subsystem 302 (and associated MLIP predictive modeling subsystem 310, MLCD predictive modeling subsystem 312, and MLProp predictive modeling subsystem 314) by passing, via combination operation 316, the resulting atomic structure output candidate model from DFT predictive subsystem 302, and, optionally, additional QPML source data 304 by way of QPML model 306, to generative machine learning subsystem 318 and iterative refinement subsystem 320 before ultimately performing uncertainty estimation and error calculation upon the predictive modeling output via predictive error computing module 322.

In one embodiment, the relaxer function of the refinement subsystem 320 may implement any one or more types of relaxation processes such as simulated annealing or other mechanisms that may relocate atoms slowly to simulate time-varying interatomic forces. Thus, this mechanism could be, in principle, any local or global optimization algorithm (gradient based or gradient-free). Typically, in various embodiments of these applications, such algorithms may be local, gradient-based optimization algorithms (e.g., the Broyden-Fletcher-Goldfarb-Shanno algorithm (BFGS algorithm), the fast inertial relaxation engine (FIRE), conjugate-gradient method, and/or quasi-Newton methods, etc.).

It is to be noted that, within the context of the present description, additional details of relaxer function subprocess components and behavior, specifically, iterative refinement subsystem 320, are addressed below with the detailed description of subsystem 400 for refining trained machine learning models for candidate atomic structures (specifically for relaxing the atomic structure to achieve greater stability).

In an embodiment, once convergence within predictive error computing module 322 has been realized and uncertainty estimation and error threshold measurements are recorded below specified error levels, the GAD application system may compile a final collection of one or more candidate generative model atomic structure composition(s) expected to be present in the desired associated properties of a viable material. That is, if the model error for a candidate material configuration (e.g., atomic structure) is below the chosen error threshold, then the model is promoted to be included into refined output dataset 324.

In a subsequent embodiment, the selected <n> candidate atomic structures may be passed, again, through validation DFT modeling 326 and/or quantum modeling for even further validation (e.g., by way of generating their formation energy and/or confirming material properties). Through this process, the best <n> candidate material configurations may be selected based on a goodness factor (including likelihood of success, etc.) when comparing any number of candidate material configurations. In some embodiments, <n> may be an integer provided by a user/expert. For example, in one embodiment, if materials that minimize formation energy are designed, where n might be desired to be 20, 20 materials may be drawn from the phase 3 dataset based on whichever of those 20 materials have the lowest predicted formation energy. Thus, with an understanding that there may be a limited number of best candidates to be selected and promoted to undergo accurate simulation using validation DFT modeling 326, it should be noted that this technique is many orders of magnitude more efficient than alternative approaches (e.g., where a nearly unlimited number of candidates of unknown promise are subjected to further simulation).

In one embodiment, the resulting final output dataset(s) of novel materials 328 may be yet further pared down to a subset of <n> novel material configurations prior to actual physical synthesis and fabrication 330. In practice, the final datasets of novel atomic structures (e.g., unit cells, periodic atomic structures, material configurations, etc.) considered to be best for one or more macro properties, may be placed in the final output dataset along with their associated charge density values, interatomic forces, global energy values, and global properties, wherein characteristics of the foregoing novel materials are known to be accurate, at least in that they have computed to the accuracy level of validation DFT processing. Further, foregoing quantum modeling may also be used to calculate and/or validate various quantum properties of selected materials.

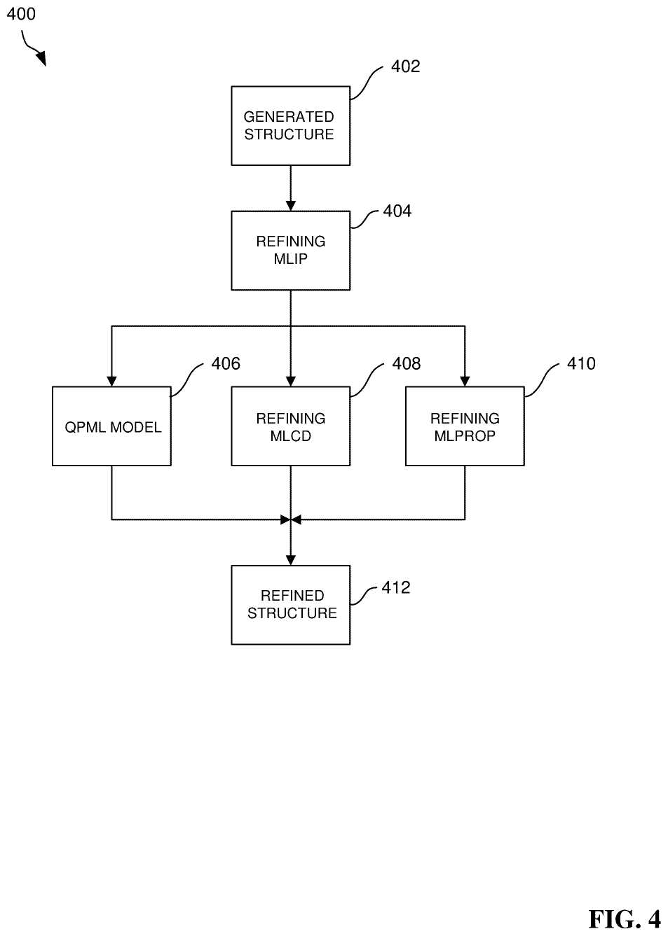

FIG. 4 illustrates a subsystem 400 for refining trained machine learning models for candidate atomic structures, in accordance with one embodiment. As an option, the system 400 may be implemented in the context of any one or more of the embodiments set forth in any previous and/or subsequent figure(s) and/or description thereof. Of course, however, the system 400 may be implemented in the context of any desired environment. Further, the aforementioned definitions may equally apply to the description below.

Within the context of FIG. 3, the subsystem 400 is a detailed look at the refinement subsystem 320, including subcomponents and steps which may be taken to further refine the atomic structure.

As shown, a generative atomistic design (GAD) application system may perform one or more relaxer functions to adjust the position(s) of the one or more atoms within a generated atomic structure. In practice, the generated structure model output 402 may commence based on output from the generative machine learning subsystem 318. This data may be used as the source data for MLIP refining subsystem 404, which may further refine the dataset(s) through, optionally, QPML refining modeling 406, MLCD refining subsystem 408, and MLProp refining modeling subsystem 410.

In an embodiment, the process of relaxing an atomic structure may allow the atoms and/or molecules to settle into their locally lowest energy configurations, which may often correspond to the manner in which they would naturally arrange themselves in the physical world. It should be noted that the process(es) may be repeated by feeding the relaxed structure to an MLIP predictor (used to predict the charge density of a relaxed configuration of an atomic structure at any point during the simulation) and continuing the process of relaxation until the maximum force on any atom is below a known stable maximum force threshold value.

When the maximum force on any atom is below a known stable maximum force threshold value, the now refined structure 412 may be passed back through predictive error computing module 322 (and continue as discussed within the context of the system 300).

It is to be noted that, within the context of the present description, the entirety of this relaxer function subprocess (subsystem 400 for refining trained machine learning models for candidate atomic structures) may take place within iterative refinement subsystem 320 of FIG. 3 for outputting trained and refined generative machine learning models for candidate atomic structures.

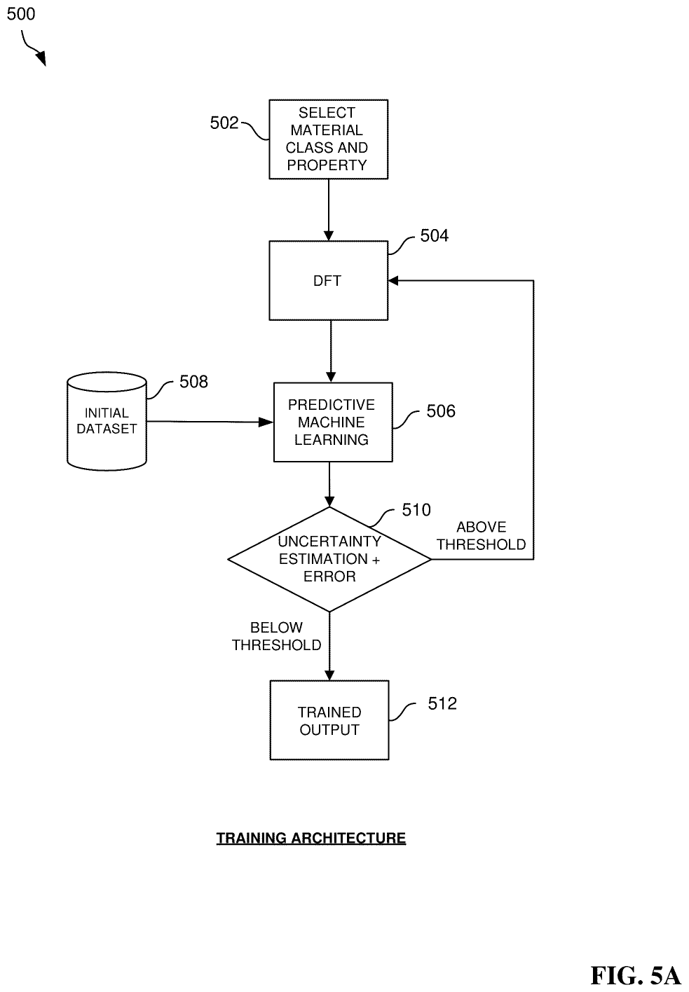

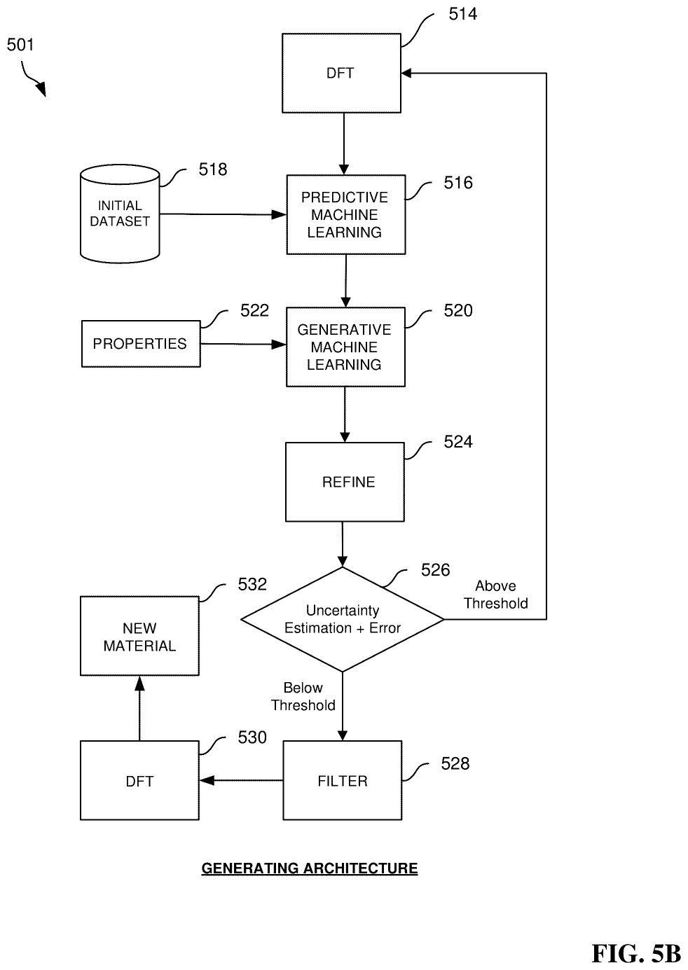

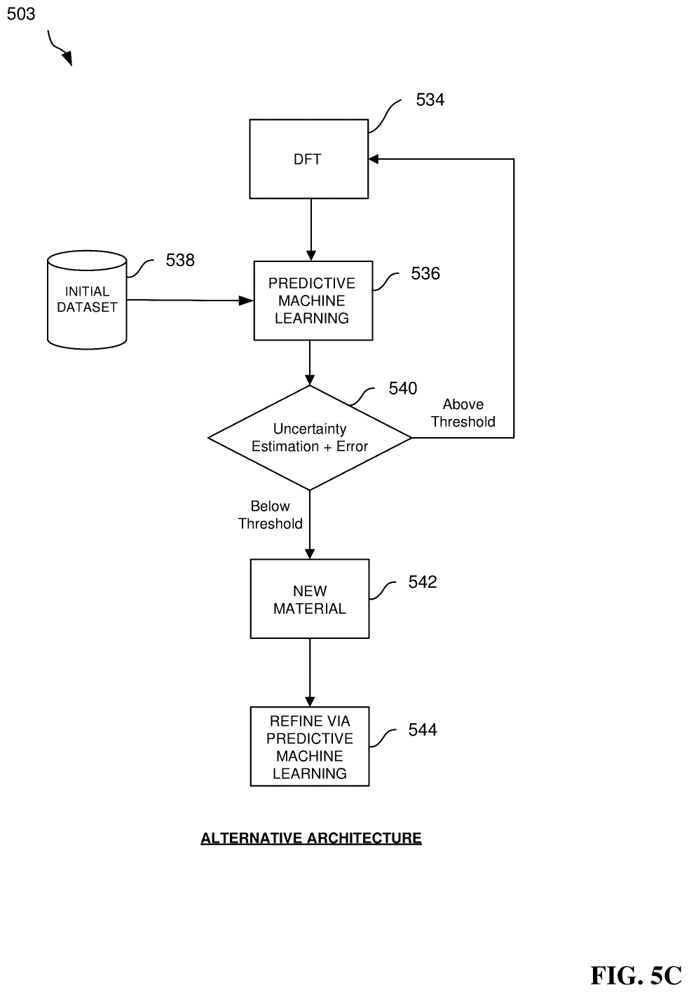

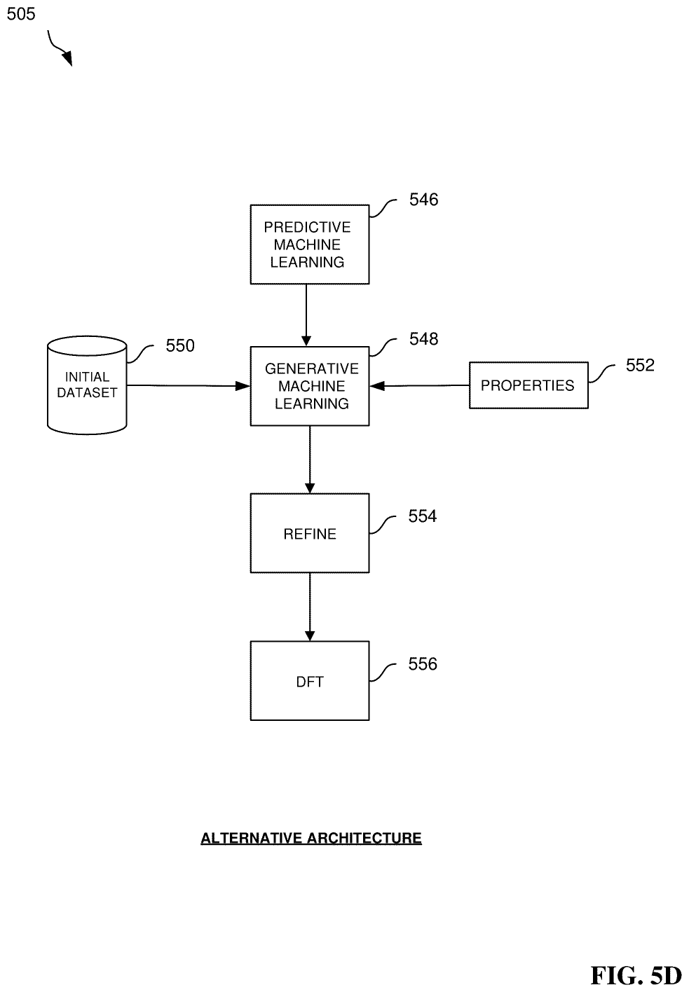

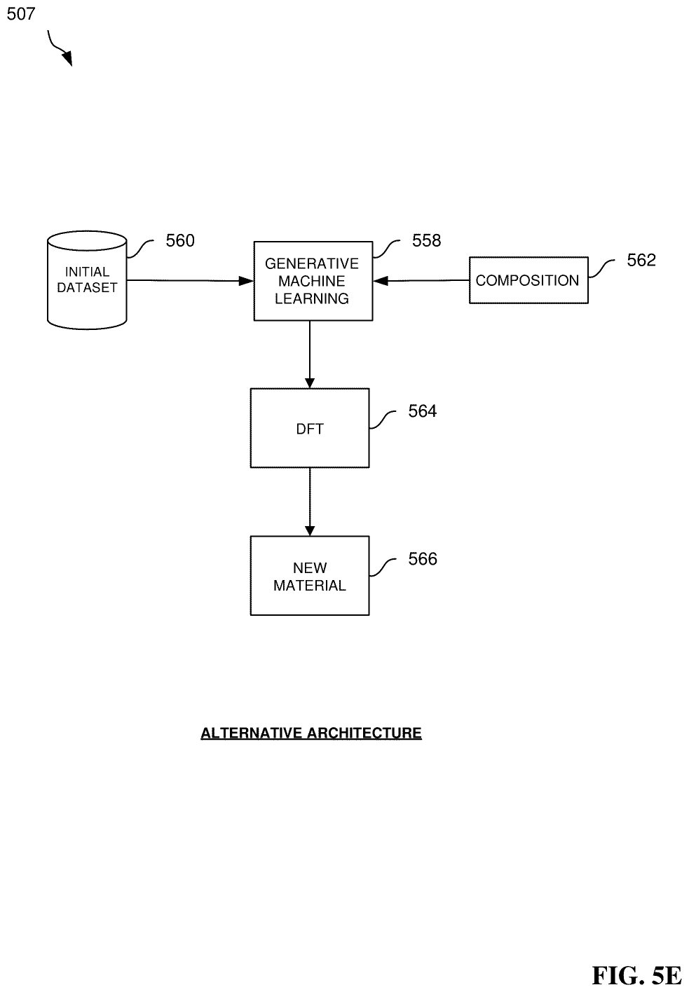

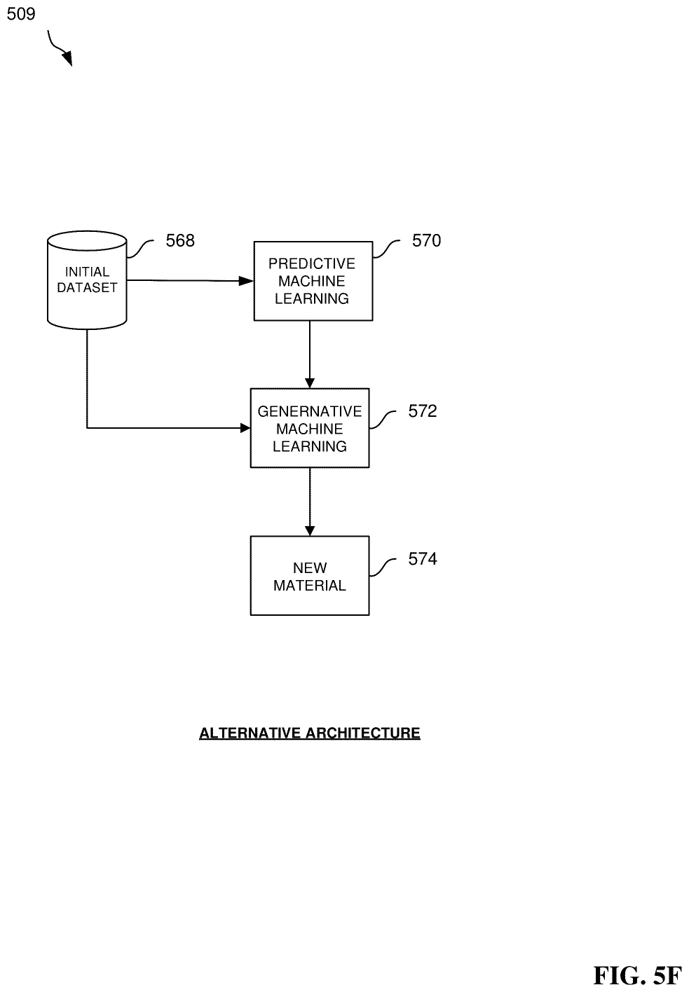

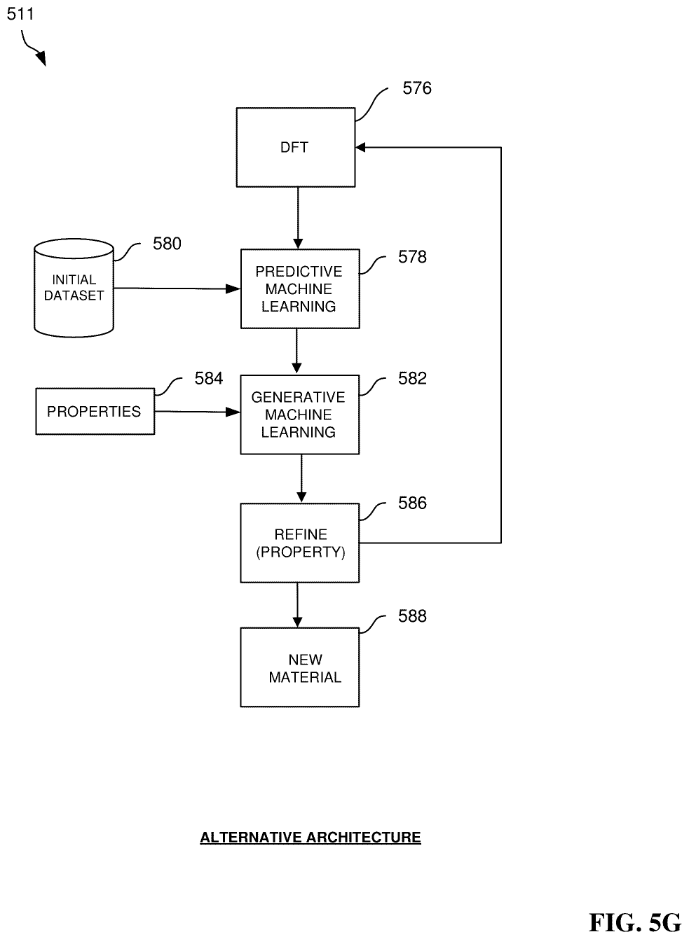

FIG. 5A illustrates a training architecture 500 for material generation, in accordance with one embodiment. As an option, the architecture 500 may be implemented in the context of any one or more of the embodiments set forth in any previous and/or subsequent figure(s) and/or description thereof. Of course, however, the architecture 500 may be implemented in the context of any desired environment. Further, the aforementioned definitions may equally apply to the description below.

As shown, the training architecture 500 is used, in one embodiment, as a training architecture for training a machine learning system.

In terms of flow, the architecture 500 shows that a material class and property are selected. See operation 502. DFT is used to understand and predict the properties of the materials with the selected material class and property. See operation 504. After the DFT analysis, predictive machine learning may be used, in combination with input from an initial dataset 508. See operation 506. It is then determined whether an uncertainty estimation and error are above or below an error threshold. See decision 510. If it is above the error threshold, the architecture 500 returns back to DFT (operation 504) to reanalyze the data and the machine learning models of the predictive machine learning (operation 506) may be used to reassess the data. Once the uncertainty estimation and error are below an error threshold, a trained output is provided. See operation 512.

FIG. 5B illustrates a generating architecture 501 for material generation, in accordance with one embodiment. As an option, the architecture 501 may be implemented in the context of any one or more of the embodiments set forth in any previous and/or subsequent figure(s) and/or description thereof. Of course, however, the architecture 501 may be implemented in the context of any desired environment. Further, the aforementioned definitions may equally apply to the description below.

As shown, the training architecture 501 is used, in one embodiment, as a generating architecture for training a machine learning system. The architecture 501 builds upon the architecture 500. In another embodiment, one purpose of the architecture 500 may be to have a sufficiently robust (and correct) dataset that materials can then be generated using the architecture 501. In other words, the architecture 500 ensures that the machine learning system is properly trained in terms of processing datasets properly and in a manner that coincides with a DFT validated output. Thus, the architecture 501 begins with the premise that the machine learning system has been properly trained.

In terms of flow, the architecture 501 shows analysis by DFT. See operation 514. It is to be appreciated that the trained output (per operation 512) may be used as the source data for the DFT analysis (per operation 514), as well as machine learning models via the predictive machine learning (per operation 516). In one embodiment, the trained output (per operation 512) may be the initial dataset 518. The generative machine learning may then be used to generate new structures (per operation 520). In one embodiment, quantum data (such as via quantum machine learning modeling) may be received via properties 522. Further, the atomic structure may be refined (per operation 524). As discussed herein, the refinement step is shown in greater detail via FIG. 4. An uncertainty estimation and error determination is made (per decision 526) such that if an error threshold is surpassed, the atomic structure and/or dataset is passed back to DFT (per operation 514) for further processing. When the determination (per decision 526) is below the error threshold, the resulting collection of atomic structures may be further filtered (per operation 528). A subset of the atomic structure dataset is then passed on to DFT for further validation and verification. See operation 530. New materials that are validated via DFT may then be outputted. See operation 532.

In taking a step back and evaluating the training architecture of FIG. 5A and/or the generating architecture of FIG. 5B, it is noted that with the two phases of training and generating, based on initial data, the system is trained using predictive machine learning models (such as MLIP, MLCD, and/or MLPRop, etc.) and then evaluated using uncertainty quantification and active learning. Based on the training, the input data can then be used in a generative model, where the predictive machine learning models (such as MLIP, MLCD, and/or MLPRop, etc.) may be used to refine the output. Such a generation of materials may be made more robust by implementing an active learning cycle, and then validating the output by DFT.

It is to be appreciated that FIGS. 5C-5G described hereinbelow represent alternative arrangements, architectures, and flows compared to the training architecture of FIG. 5A and/or the generating architecture of FIG. 5B. It is to be recognized that FIGS. 5C-5G may be supplemented with parts, flows, and/or components as needed with elements from the training architecture of FIG. 5A and/or the generating architecture of FIG. 5B.

FIG. 5C illustrates an alternative architecture 503 for material generation, in accordance with one embodiment. As an option, the architecture 503 may be implemented in the context of any one or more of the embodiments set forth in any previous and/or subsequent figure(s) and/or description thereof. Of course, however, the architecture 503 may be implemented in the context of any desired environment. Further, the aforementioned definitions may equally apply to the description below.

As shown, the architecture 503 represents an assembly where training and generation is in a single flow. DFT is used to understand and predict the properties of the materials with the selected material class and property. See operation 534. Such information from DFT, as well as an initial dataset 538 are provided to predictive machine learning (operation 536), where machine learning models (such as MLIP, MLCD, MLProp) may be applied to predict how the atomic structure may function and respond. It is then determined whether an uncertainty estimation and error are above or below an error threshold. See decision 540. If it is above the error threshold, the architecture 503 returns back to DFT (operation 534) to reanalyze the data and the machine learning models of the predictive machine learning (operation 536) may be used to reassess the data. Once the uncertainty estimation and error are below an error threshold, a new output is provided. See operation 542. After the new material is outputted, it may then be further refined via predictive machine learning (operation 544) such as by the machine learning models (such as MLIP, MLCD, MLProp, etc.).

In comparing the architecture 503 to the training architecture of FIG. 5A and/or the generating architecture of FIG. 5B, the architecture 503 represents a simplified architecture. In one embodiment, the architecture 503 may represent a brute force method where atomic forces and/or properties (i.e. structure numeration) are evaluated to determine the likelihood of success of a novel structure. Thus, the architecture 503 may be made more robust by using generative models to replace a brute force approach. Further, the addition of multiple machine learning models (such as two models rather than a single model) may be used to increase the accuracy of the resulting novel material.

FIG. 5D illustrates an alternative architecture 505 for material generation, in accordance with one embodiment. As an option, the architecture 505 may be implemented in the context of any one or more of the embodiments set forth in any previous and/or subsequent figure(s) and/or description thereof. Of course, however, the architecture 505 may be implemented in the context of any desired environment. Further, the aforementioned definitions may equally apply to the description below.

As shown, the architecture 505 represents an assembly where training and generation is in a single flow. Predictive machine learning (operation 546) may be used as a starting point for evaluating datasets. Such datasets may then be provided to the generative machine learning 548 to generate new potential structures. The generative machine learning 548 may receive input from an initial dataset 550 and properties 552 (including but not limited to physical properties, quantum data properties, etc.). Further, an output of the generative machine learning 548 is then refined (operation 554) where the atomic structure may be analyzed to determine if the arrangement can be relaxed and/or modified to achieve greater stability. Further, an output from refine (operation 554) can the be validated via DFT 556.

In comparing the architecture 505 to the training architecture of FIG. 5A and/or the generating architecture of FIG. 5B, the architecture 505 represents a simplified architecture. In one embodiment, the architecture 505 may include taking an initial data set and training a generative model, which in turn, may be refined using at least one predictive machine learning model (such as MLIP, MLCD, MLProp, etc.). It is to be understood that use of multiple machine learning models may assist in making the resulting prediction (for novel material) more accurate and stable. Such an architecture 505 likewise may be made more robust by having an active learning cycle and/or including quantum data in the determination of novel material.

FIG. 5E illustrates an alternative architecture 507 for material generation, in accordance with one embodiment. As an option, the architecture 507 may be implemented in the context of any one or more of the embodiments set forth in any previous and/or subsequent figure(s) and/or description thereof. Of course, however, the architecture 507 may be implemented in the context of any desired environment. Further, the aforementioned definitions may equally apply to the description below.

As shown, the architecture 507 represents an assembly where training and generation is in a single flow. An initial dataset 560 and composition 562 (of structure, of desired properties of material, etc.) may be provided to generative machine learning (operation 558) to generate new materials. An output from the generative machine learning (operation 558) may then be evaluated using DFT (operation 564) resulting in new material (operation 566).

In comparing the architecture 507 to the training architecture of FIG. 5A and/or the generating architecture of FIG. 5B, the architecture 507 represents a simplified architecture that focuses primarily on using generative models. In one embodiment, the architecture 507 may use a dataset to train the generative model, which in turn is validated with DFT. Such an architecture 507 may be made more robust by using predictive machine learning models (such as MLIP, MLCD, MLProp, etc.), using a property predictor, and/or quantum data. Further, the architecture 507 may be modified to include an active learning cycle and a refinement step.

FIG. 5F illustrates an alternative architecture 509 for material generation, in accordance with one embodiment. As an option, the architecture 509 may be implemented in the context of any one or more of the embodiments set forth in any previous and/or subsequent figure(s) and/or description thereof. Of course, however, the architecture 509 may be implemented in the context of any desired environment. Further, the aforementioned definitions may equally apply to the description below.

As shown, the architecture 509 represents an assembly where training and generation is in a single flow. An initial dataset 568 may be provided to predictive machine learning (operation 570) to be analyzed via machine learning models (such as MLIP, MLCD, and/or MLProp) and generative machine learning (operation 572) to generate new materials. An output new material (operation 574) may result from the generative machine learning (operation 572).

In comparing the architecture 509 to the training architecture of FIG. 5A and/or the generating architecture of FIG. 5B, the architecture 509 represents a simplified architecture that focuses on property prediction. Such an architecture 509 can be supplemented, at a minimum, with an active learning cycle, a refinement step and/or validation step of the output from the generative model, and/or a conditional generation step.

FIG. 5G illustrates an alternative architecture 511 for material generation, in accordance with one embodiment. As an option, the architecture 511 may be implemented in the context of any one or more of the embodiments set forth in any previous and/or subsequent figure(s) and/or description thereof. Of course, however, the architecture 511 may be implemented in the context of any desired environment. Further, the aforementioned definitions may equally apply to the description below.

As shown, the architecture 511 represents an assembly where training and generation is in a single flow. DFT is used to understand and predict the properties of the materials with the selected material class and property. See operation 576. Such information from DFT, as well as an initial dataset 580 are provided to predictive machine learning (operation 578), where machine learning models (such as MLIP, MLCD, MLProp) may be applied to predict how the atomic structure may function and respond. The generative machine learning (operation 582) may receive input from the predictive machine learning (operation 578) as well as from properties 584 (including but not limited to physical properties, quantum data properties, etc.). Further, an output of the generative machine learning (operation 582) is then refined (operation 586) where the atomic structure may be analyzed to determine if the arrangement can be relaxed and/or modified to achieve greater stability. An active learning cycle may loop results from the refine (operation 586) back to DFT (operation 576), which may result in a new material (operation 588).

In comparing the architecture 511 to the training architecture of FIG. 5A and/or the generating architecture of FIG. 5B, the architecture 511 represents a simplified architecture that focuses primarily on composition of the material (and not as much on the structure of the material). Such an architecture 511 can be supplemented, at a minimum, with having a quantum data model (such as a quantum probablistic machine learning model) and an uncertainty quantification to determine whether the resulting material is below an error threshold.

As indicated above, it is to be appreciated that FIGS. 5C-5G described herein represent alternative arrangements, architectures, and flows compared to the training architecture of FIG. 5A and/or the generating architecture of FIG. 5B. In comparing globally FIGS. 5C-5G to the training architecture of FIG. 5A and/or the generating architecture of FIG. 5B, a few key points may be made. The architecture used to predict generative materials may be made generally more robust (i.e. more accurately predicted and generated novel materials) by: 1) including an active learning cycle based on the generative machine learning model; 2) including multiple predictive machine learning models (and in particular a charge density model such as MLCD); and/or 3) including quantum data (and Quantum Probabilistic Machine Learning) in the process of generating and/or refining atomic structures. As such, predictive machine learning, generative machine learning, quantum probabilistic machine learning, DFT, experimentation, and active learning may be used in part or collectively to generate new atomic structures for materials. Further, the elements used may be selectively integrated as needed to create novel materials with desired properties. It is to be appreciated that other elements of FIGS. 5A and/or 5B (or any of the other figures disclosed herein) may be integrated and used as needed within the context of FIGS. 5C-5G.

In one embodiment, the FIGS. 5C-5G represent a variety of other configurations, which may be appropriate for a given context, depending on the novel material that is to be generated. Thus, a context of the novel material may dictate the exact architecture and arrangement needed in order to produce a novel material conforming to material specifications. As such, a flexible and variety of arrangements is feasible based on the disclosure herein.

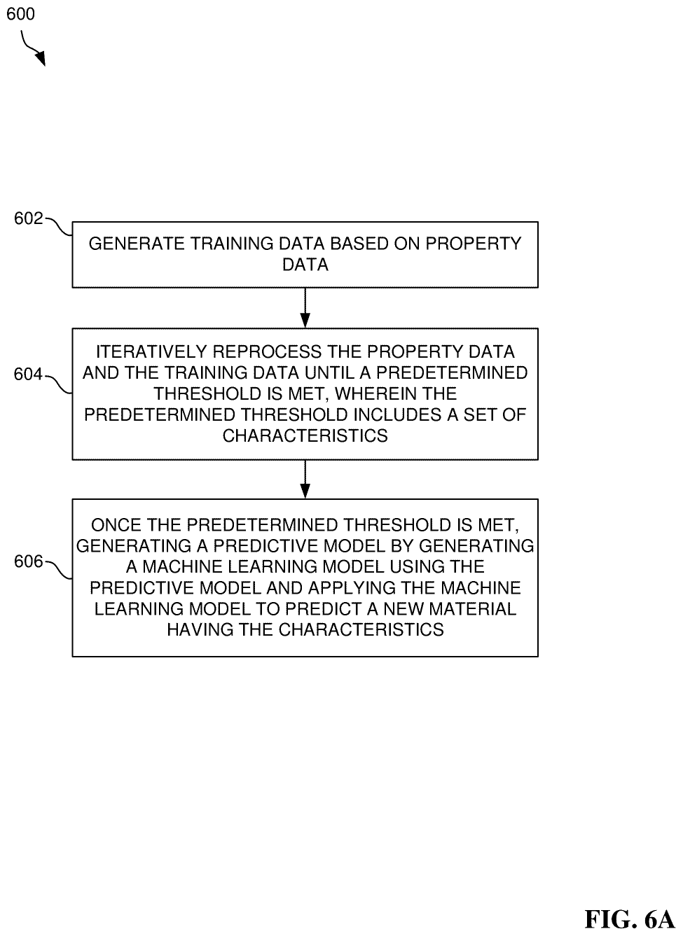

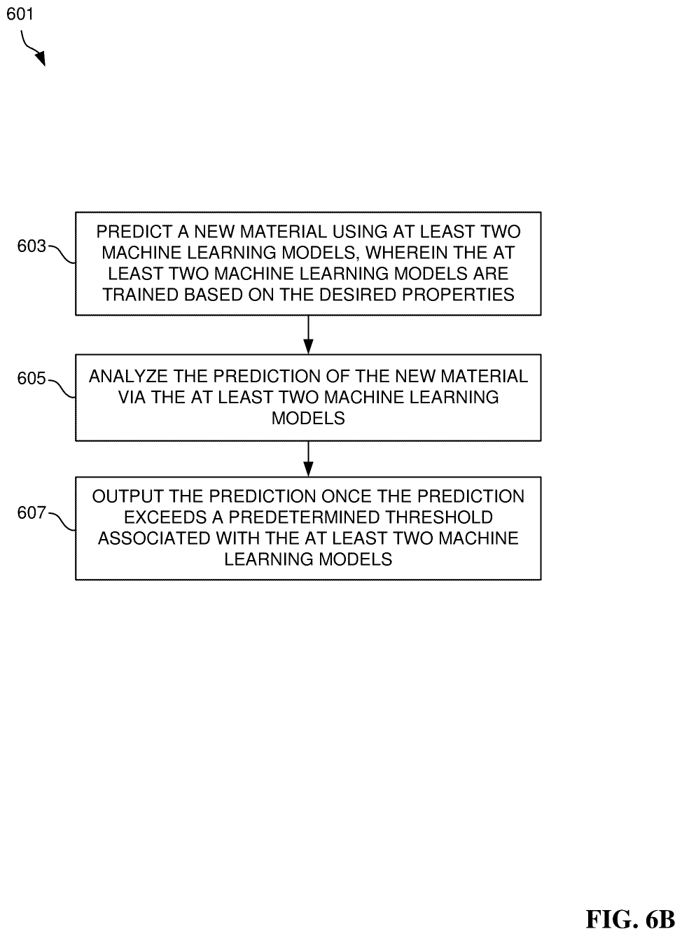

FIG. 6A illustrates a method 600 for creating an active learning cycle using at least two machine learning models, in accordance with one embodiment. As an option, the method 600 may be implemented in the context of any one or more of the embodiments set forth in any previous and/or subsequent figure(s) and/or description thereof. Of course, however, the method 600 may be implemented in the context of any desired environment. Further, the aforementioned definitions may equally apply to the description below.

As shown, the method 600 may generate training data based on property data. See operation 602. Additionally, the method 600 may iteratively reprocess the property data and the training data until a predetermined threshold is met, where the predetermined threshold includes a set of characteristics. See operation 604. Further, the method 600 may, once the predetermined threshold is met, generate a predictive model by generating a machine learning model using the predictive model and applying the machine learning model to predict a new material having the characteristics. See operation 606.

In comparing the method 600 to the method 1B00, it is noted that the method 600 specifically highlights, in one embodiment, a reiterative nature of processing the property data and training data until the predetermined threshold is met. In other words, there is a greater emphasis on the active learning cycle that may be used by the system.

In one embodiment, the desired material and properties of the desired material may be received and analyzed using one or more DFT models. As such, DFT may be used as a computational approach for creating simulations of novel materials that fulfill specific needs. For example, computational methods may be used within a DFT paradigm to help facilitate the process of experimenting with atomic structures. In addition, active learning models may be trained by reiteratively applying a combination of two or more machine learning subsystems, including, but not limited to MLIP, MLCD, MLProp, and QPML subsystems. Further, the resulting uncertainty estimates and error calculations may be evaluated to determine whether additional training may be necessary to achieve optimal active learning model creation and output a trained model for generative purposes.

In another embodiment, at least two of a MLIP, MLCD, and MLProp may each compute analytical models based on training data from the DFT subsystem. Further, once the training data has been analyzed and/or adjusted, the method 600 may again go through additional machine learning simulation. Additionally, method 600 may then verify whether or not the output atomic structure sample is below or above the predetermined threshold, where the system may double check the atomic structure from the DFT subsystem.

FIG. 6B illustrates a method 601 for predicting a new material with desired properties, in accordance with one embodiment. As an option, the method 601 may be implemented in the context of any one or more of the embodiments set forth in any previous and/or subsequent figure(s) and/or description thereof. Of course, however, the method 601 may be implemented in the context of any desired environment. Further, the aforementioned definitions may equally apply to the description below.

As shown, the method 601 may predict a new material using at least two machine learning models, where the at least two machine learning models are trained based on the desired properties. See operation 603. Additionally, the method 601 may analyze the prediction of the new material via the at least two machine learning models. See operation 605. Further, the method 601 may output the prediction once the prediction exceeds a predetermined threshold associated with the at least two machine learning models. See operation 607.

In comparing the method 601 to the method 1B00, it is noted that the method 601 specifically highlights, in one embodiment, a dependency on threshold values and whether a candidate atomic structure's composition and performance surpasses or falls short of those threshold values.

In one embodiment, the at least semi-supervised learning may use uncertainty-driven active learning. For example, criteria for training convergence based on the semi-supervised learning may be set even when, after the learning rate is sufficiently low, no further improvement in the error is observed.

In an embodiment, at least two of a MLIP, MLCD, and MLProp may each compute analytical models based on training data from the DFT subsystem. Further, quantum data (and quantum probabilistic machine learning models) may model the atomic structure data based on training data from the DFT subsystem. Further, once the training data has been analyzed and/or adjusted, the process may again go through additional machine learning simulation (hence the active learning process of the method 601). Additionally, the process may then verify whether or not the output atomic structure sample is below or above the predetermined error threshold, where the system may double check the atomic structure from the DFT subsystem.

In various embodiments, it is recognized that conventional experimental research can be expensive and time-intensive. The disclosure herein may allow for computer simulations for modeling and predicting material behavior in a manner that novel materials can be predicted, refined, outputted, and then synthesized.

FIG. 7A depicts an environment in which embodiments of the present disclosure can operate. As an option, one or more aspects shown in environment 7A00, including any instancing of the shown components, may be combined to realize one or more of the techniques described herein.

FIG. 7A discloses a generative atomistic design application (GAD application) for design of materials, where the GAD application may be implemented to execute using a generative atomistic design computation configuration 716 on a computing platform such as 718. Generative atomistic design application 724 (GAD application) for designing materials executes on at least one processing element, which may be a central process unit (CPU) and/or a graphics processing unit (GPU). In some embodiments the generative atomistic design application is further customized to execute on one or more specialized processing elements such as a neuromorphic processor, and/or an array processing unit, and/or a field-programmable gate array (FPGA) or other operational component. Furthermore, generative atomistic design computation configuration 716 itself is not limited to an implementation in any specific semiconductor technology and may be, wholly or in part, implemented using a quantum computing platform or a quantum computer or an opto-electronics based computing platform or a photonics computer or electro-optical computer.

In one embodiment, computing platform 718 comprises processing elements such as one or more CPUs (e.g., CPU1 through CPUN) and/or one or more GPUs (e.g., GPU1 through GPUK), system bus 722, and a processor memory and/or graphics memory 726. In addition to system bus 722, there may also include one or more communication links 720 or a network-on-chip (NOC) links in the computing platform to allow communication between various processing elements and also between the various processing elements and the peripheral interfaces such as connections to external storage devices, external data repository 728, and/or one or more network interface ports 730 optionally coupled to the internet 734 and communicating via one or more packets such as network protocol packet 732 using at least one communication protocol.

The generative atomistic design application 724 may be customized to run on one or more CPUs and/or one or more GPUs depending on the performance needed to achieve a certain computation throughput and the size of the problem being solved. A portion of the GAD application may run on one or more GPUs and another portion of the GAD application may run on one or more CPUs. Yet another portion of the GAD application may run on some specialized processing elements. The GAD application may comprise specialized software subsystems and it may further use some standard middleware or software components (e.g., libraries etc.) and application program interfaces (APIs) for accessing data from storage, etc. In no case, must it be construed that the GAD application contains only standard or open source or other freely available libraries and frameworks only. The GAD application is custom built for the generative design of various materials at the atomistic level with a primary focus on solid state materials.

The paradigm of using the GAD application starts with one or more users/experts seeking to design one or more materials of interest. Several inputs may be received that comprise materials requirements 702. Materials requirements 702 may further comprise specific materials with specific compositions (chemical and/or physical) in the form of material requests 704 (for example, a solid state electrolyte, an amorphous ceramic, etc.), and one or more material property requests (e.g., global property or gross property or macro property) 706 (for example, conductivity or ionic conductivity, dielectric constant, dielectric strength, etc.) and is not limited to just these properties. In the context of this disclosure, material requests could be in the form of material classes (for example, pure metals, metal alloys, semiconductors such as silicon; or germanium or compound semiconductors such as gallium arsenide or indium phosphide, polymers such as thermoplastics, thermosetting plastics, elastomers, biopolymers, ceramics such as porcelain, silicon carbide, alumina; or amorphous ceramics such as glass, composites such as fiberglass; or biomaterials such as hydroxyapatite, collagen, biodegradable polymers; or solid-state electrolytes such as beta-alumina, lithium phosphorous oxynitride; or polymer based solid electrolytes such as polyethylene oxide with lithium salt; or piezo electric materials, ferroelectric materials, or smart materials such as nitinol, electrorheological and magnetorheological fluids and gels; or high temperature materials like Inconel and titanium alloys, hybrid materials, photonic materials, amorphous metals and amorphous polymers, etc., but not limited to these alone).

The material classes may also include newer kinds of materials and known material types. Material properties requested may include one or more of several global properties such as electrical conductivity, ionic conductivity, thermal conductivity, hardness, dielectric constant, dielectric strength, corrosion resistance, reactivity, surface tension, electrical resistivity, electric susceptibility, electrostriction, permittivity, piezoelectric constants, Seebeck coefficient, hysteresis, diamagnetism, hall coefficient, magnetostriction, permeability, pyromagnetic coefficient, piezo magnetism, bulk modulus, density, ductility, elasticity, mass diffusivity, specific heat, luminosity, photosensitivity, refractive index, transmittance, photoelasticity, boiling point, melting point, thermal expansion coefficient, etc., but not limited to just these properties. Materials requirements 702 may further comprise experimental learnings 708 in the form of measurement data (e.g., tables, files, records, etc.) such as measurements deriving from surface differential reflectivity (SDR), diffuse reflectance spectroscopy (DRS), diffraction data, interference waveforms, etc. The experimental data is not limited to the properties listed in this disclosure and may, in the future, include newer properties not yet discovered or conceived of as of the filing date of this disclosure. The intended area of exploration will inform what density functional theory (DFT) computations (including any molecular dynamics simulations) or other quantum mechanical computations using techniques such as quantum many-body perturbation models (e.g., random phase approximation (RPA), GW approximation, etc.), density matrix renormalization group, dynamical mean field theory, variational quantum eigensolver (VQE), variational quantum thermalizer (VQT), etc.) will be performed to generate the initial training dataset.

Based on the new material requirements, a material expert may use one or more of available methods and tools (e.g., genetic algorithms, or Monte Carlo methods, Bayesian optimization, etc.) to generate inputs 712 one or more atomic structures (either periodic atomic structures with associated period boundary conditions or non-periodic atomic structures such as molecules), which are appropriate kernels for creating desired structures as a part of initial dataset 710. Certain instances of any initial dataset 710 may further comprise associated quantum data (for example, quantum states, Hamiltonian operators or functions, correlation functions, etc.) corresponding to the atomic structures and properties chosen and which cannot be modeled using DFT techniques. This system only works when trained on an initial dataset that is representative of a material class of interest. Therefore, the system must always start from a set of “known” materials and their associated properties. It can then learn from them and suggest newer configurations that may optimize a property or properties of interest.

In the context of this disclosure, atomic structure (sometimes also known as atomic configuration or material configuration) refers to an arrangement of atoms in matter. Atomic structures may be periodic or non-periodic. Furthermore, this atomic structure may also refer to other atomic scale structures (e.g., molecular structures, structure of ligands, etc.). An atomic structure or atomic configuration may contain information about the atomic species/atomic type and atomic location such as coordinates along with the lattice vectors, which define their periodic boundary conditions (PBCs). In the context of this disclosure, a periodic boundary condition associated with an atomic structure is a simulation artifice to represent a “sea-of-atoms” (e.g., see sea-of-atoms 792 in FIG. 7F), where the atomic structure is imagined to repeat indefinitely in one or more directions. To simulate a finite subset of a sea-of-atoms, a non-periodic atomic structure that repeats may be coupled with a set of boundary conditions in a unit cell (comprising lattice vectors that define periodic boundary conditions of the atomic structure) to create a periodic atomic structure for simulation. The set of boundary conditions (e.g., periodicity of location information, forces, bonds, etc.) are called the periodic boundary conditions (PBCs). The atomic structure (e.g., the unit cell 794 in FIG. 7F) may comprise a non-periodic atomic structure 790 along with the associated periodic boundary conditions. In some embodiments, different representations of atomic structures 714 can equivalently capture the boundary conditions of the same periodic atomic structures.

Atomic structures are foundational in materials science, as a particular atomic structure controls a material's properties, behaviors, and the material's functionalities. Whether it is a simple arrangement like a crystal lattice in metals or a more complex configuration in amorphous materials, the atomic structure is the embodiment from which all global/macroscopic material properties emerge.

The generative atomistic design application 724 in 7A receives several inputs that may comprise initial dataset 710 comprising one or more atomic structures 714, corresponding or associated quantum data, etc. The GAD application is configured to execute in phases. Strictly as an example, a phase 0 might be configured to prepare, create, load, and/or receive one or more models of materials starting from atomic structures 714 (e.g., periodic atomic structures), and/or quantum data, and/or atom types, and/or atom locations, etc., whereas further phases are configured to perform operations as may be required for additional machine learning materials and/or additional model training and/or for making additional predictions or inferences pertaining to any or all of interatomic forces, global energies, interatomic potential, charge densities, global properties, quantum properties, material configuration/atomic structure generation, atomic structure refinement, material selection, etc.

In one embodiment as shown in 7A, the GAD application (e.g., generative atomistic design application 724) comprises subsystems for 5 phases:
Phase 0 is a dataset creation/loading phase implemented using dataset creation/loading subsystem 740, which receives initial dataset 710 that may be taken in as phase 0 input dataset 739, in any appropriate format, and added to any existing instances of phase 0 input datasets. The updated instances of phase 0 input datasets are then used to load and/or create further datasets (for example, phase 0 output dataset in 742) using one or more DFT-based tools for use in other phases.
Phase 1 is a machine learning model training phase implemented using machine learning model training subsystem 744 controlled by a training manager that uses a training dataset (e.g., phase 0 output dataset 742) to train one or more machine learning predictive material models (such as multi-layer perceptron 7100).
Phase 2 is a dataset generation phase (controlled by a dataset prediction manager) using predictive machine learning and implemented using prediction subsystem 750 that uses the material models trained in Phase 1 (e.g., trained machine learning models 749) along with phase 2 input dataset 748 comprising atomic structures, quantum data, etc., for inference/prediction/output of phase 2 datasets (e.g., phase 2 prediction subsystem 750).
Phase 3 is a materials generation phase implemented using material generation subsystem 754 that receives input dataset 752 comprising multiple atomic structures (or atomic structures with PBCs) along with the corresponding/associated charge densities, and/or one or more predicted properties (i.e., property values) associated with each of the corresponding periodic atomic structures. The material generation subsystem trains a generative machine learning model to generate new material configurations comprising unit cells, and/or periodic unit cells, and/or other representations of atomic structures.
Phase 4 is a refinement phase (e.g., refinement subsystem 756) in which the material configurations (atomic structures, unit cells, etc.) generated by a generative machine learning model are consumed by refinement subsystem 756 that refines the generated structures and associated charge density and property values to relax the atomic structures to make them stable by adjusting the locations of atoms so as to reduce interatomic forces inside of them and create refined datasets (e.g., refined output dataset 769).
Phase 5 is a material selection and validation phase that is implemented using material selection and validation subsystem 760, which takes in a refined dataset (e.g., refined output dataset 769) and selects one or more candidate materials and their corresponding/associated data, which are stored in a final output dataset (e.g., final output dataset 762). The material properties and charge densities of the candidate materials may be accurately computed in this phase using the density functional theory tools and models. In addition to the foregoing candidate materials as computed in this phase, other properties and/or any aspect of a material and/or its simulation model counterparts can be stored in final output dataset 762.
Any aspects of any operational element of any of the foregoing phases of computation within the generative atomistic design application can be controlled via a user interface (e.g., user interface handler 766), and/or via an application programming interface (e.g., API I/O manager 764).

Machine learning (ML) is a subset of techniques of artificial intelligence that involves teaching computers to identify patterns and make decisions from data without being explicitly programmed for specific tasks. In the context of this disclosure, and in the context of materials science, machine learning can be employed to predict material properties, to predict material behaviors, and to design new materials. By analyzing precomputed data (e.g., including or based on atomic structures and/or including or based on observed or calculated system dynamics and/or including or based on observed or calculated material properties), machine learning algorithms can efficiently identify relationships that might be absent (or obscured) when using traditional analytical methods. This offers the potential to accelerate the discovery of innovative materials, optimize manufacturing processes, and enhance the understanding of complex material behaviors. Machine learning algorithms may be implemented using any of the generally known methods. However, the customization of some of the generic methods for specialized applications in the context of the GAD application for generation of new materials is merely one of the many subjects of this disclosure.

### Phase 0: Dataset Creation Phase

In order to support the various execution phases, the generative atomistic design application 724 uses several APIs, data structures, and static or constant data and functions, which may be distributed into and/or accessible by several subsystems. One of these subsystems, used in phase 0 of the dataset creation phase, is a density functional theory subsystem that supports density functional theory (DFT) tools and functions (e.g., the shown DFT tools 741). The density functional theory subsystem uses density functional theory as is known in the study of physics, chemistry, or materials science to compute datasets (e.g., phase 0 output dataset 742) that comprise electronic structures and properties of atomic configurations (e.g., atomic structures, charge densities, global energy, interatomic forces, global properties, etc.) The dataset creation phase optionally may also use one or more subsystems for creating or loading quantum properties, models, and functions that optionally provide quantum property models and values to the phase 0 output dataset 742. Phase 0 output dataset 742 and phase 0 input dataset 739 may be used by later phases such as Phase 1 through Phase 5, and/or by their related subsystems (e.g., predictive material model training subsystem 744, prediction subsystem 750, material generation subsystem 754, a refinement subsystem 756 and/or validation subsystem 760), as and when requested.

Density functional theory (DFT) is a computational approach used in physics, chemistry, or material sciences to investigate material properties, and more often, the electronic properties of many-body systems, like atoms, molecules, ligands, ions, and/or solids. Instead of trying to track each electron's movement (or each hole's movement in solid-state materials, for example, semiconductors) or each ion's movement, which can very complex both physically and computationally, DFT focusses on the overall electron density in one such case (e.g., how electrons are distributed in space) and simplifies the problem, which makes it feasible to predict how atoms, molecules, etc. behave in configurations—thereby helping the design of new materials—as well as chemical reactions. Although DFT focusses on simplifying the many-body problem mentioned above, DFT is still computationally very expensive. The present disclosure uses machine learning methods and subsystems to further speed up the vast number of computations needed to design materials at the atomic scale.

One aspect of the GAD application is to use predictive machine learning to speed up computations that would otherwise be done by a density functional theory (DFT) subsystem and/or quantum property calculation methods (e.g., quantum many-body perturbation theory (e.g., RPA, GW), density matrix renormalization group, dynamical mean field theory, variational quantum eigensolver (VQE), and variational quantum thermalizer (VQT), etc.), which are computationally expensive. In the context of this disclosure, predictive machine learning (used in some phases of the GAD application) is a subset of machine learning (e.g., supervised machine learning) where machine learning models (e.g., machine-learned surrogate models) are trained to make predictions about future or unseen data based on patterns identified from historical or known data. The primary goal is to output specific values or classifications based on input features. For instance, in the context of this disclosure, in materials science, predictive machine learning might be used to predict the conductivity, strength, or melting point of a material based on its atomic structure.

One further subsystem used in GAD application includes the in-memory data, functions, and APIs related to one or more models trained on material classes and properties that comprises a set of numeric weight values associated with pairs of “artificial neurons” in one or more layers, which may be selected for further use in the various phases of the generative atomic design application simulation. At least one material class and properties model could be related to material requests 704 and property requests 706.

Some embodiments may train (e.g., for the purpose of quantum property learning (QPL)), quantum property learning model 755. Such learned quantum properties (e.g., quantum property Q-Prop 757A through quantum property Q-Prop 757M) may be included in the GAD application. This QPL model can be used, for example, when a material structure needs to be refined using certain quantum properties.

In order to initiate a workflow, a material class may be selected and at least one associated property to target material generation. In one example embodiment, an inorganic material for use as a battery's solid-state electrolyte with high ionic conductivity may be targeted. In this case, the material class would be “solid-state electrolyte” and an associated property would be “ionic conductivity.” This information is used to inform the use of DFT to create a dataset for use in one or more of the following phases.

The dataset creation phase of the simulation (phase 0 portion of the GAD application simulation) outputs as a part of the dataset creation phase output into (1) one or more representations of atomic structures, (2) one or more atomic species and coordinates, (3) one or more corresponding/associated periodic boundary condition models (if any) comprising lattice vectors, and (4) one or more corresponding/associated interatomic force models. Some or all of the foregoing may include characterization of forces on each individual atom, and/or one or more corresponding global energy values, and/or one or more corresponding volumetric charge density values along with a set of other simulation parameters including any of a variety of properties (e.g., macro or gross properties and/or quantum properties, etc. as may be identified or selected by a user/expert. The dataset creation phase output dataset is used in the machine learning model training phase and/or it may be used in the dataset generation/prediction phase and/or any other phase that may receive it and use it as needed.

### Phase 1: Machine Learning Model Training Phase

In the embodiment shown in FIG. 7B, the machine learning model training phase is used for materials learning, and is implemented using machine learning model training subsystem 744. Machine learning model training subsystem 744 receives the dataset creation phase output (for example, phase 0 output dataset) and, in response to the received dataset, machine learning model training subsystem 744 trains the machine learning model for materials learning.

Machine learning model training subsystem 744 further comprises a machine learning interatomic potential model training subsystem (e.g., MLIP model training subsystem 745, which is controlled by a training manager. As shown, the MLIP model training subsystem further comprises an atomic structure model training capability, which receives as input one or more atomic structures, unit cells, PBCs, (e.g., unit cell values 746, etc.) from the input dataset (e.g., phase 0 output dataset created by DFT tools), and optionally, by quantum tools 743)), which in turn is used to train one or more periodic atomic structure models inside the MLIP model. In some embodiments, a portion of the machine learning interatomic potential model may be trained using one or more unit cells and/or periodic atomistic structures as received in an input dataset.

In the context of this disclosure the term an “atomic structure” may refer to a structure of atoms or molecules, along with associated periodic boundary conditions, and is not limited to these alone (e.g., ions, ligands, etc., may also be included).

MLIP model training subsystem 745 may receive as input, one or more periodic boundary conditions (PBCs) along with the associated atomic structures in the input dataset as applicable to the corresponding/associated atomic structures. The atomic structures' model training capability includes consideration of periodic boundary conditions so as to train one or more periodic boundary conditions (e.g., see the illustration of a unit cell 794 in FIG. 7F) that allow simulation to proceed on a limited size atomic structure. The results of such a simulation can be interpreted to repeat, subject to the constraints of any applicable periodic boundary conditions.

In the context of this disclosure, periodic boundary conditions (PBCs) comprise parameters, techniques, tools and methods used in simulations where a system of atoms or molecules, etc., (e.g., a group of atoms) is configured to appear to repeat indefinitely in all directions. By using PBCs, researchers can simulate a small portion of a larger system while effectively mimicking the behavior of a much larger system, or an infinite system. This technique is especially useful in reducing computational costs while still capturing the essence of large-scale phenomena.

The MLIP model training subsystem 745 may further comprise global energy value training capability that is used to train the MLIP model along with the periodic atomic structures. MLIP is trained to model the potential energy surface or the interatomic potential that can be further used to conduct structural optimizations (for example, energy minimization computations) or run long range/larger scale molecular dynamics simulations.

MLIP model training subsystem 745 further comprises interatomic force model training capability 747 that uses at least one interatomic force model in the training phase to compute the interatomic potentials over one or more atomic structures. An illustration to visualize interatomic forces is an interatomic force in a material configuration (e.g., see interatomic force model 796 in FIG. 7F), where the arrows indicate forces exerted on one another by atoms. Interatomic force models are often parameterized on data that ignores ultrashort-ranged interactions and very long-ranged interactions, and the physics of a core wavefunction overlap may not be included in the model. In one explanation of the art, interatomic forces are the forces between the atoms of a molecule and/or an atomic structure due to the electrostatic force between the nuclei and electrons of atoms, and between the nucleus of one atom and the electrons of the other atom or atoms. In some embodiments an interatomic force may be obtained by taking a derivative of the total energy with respect to the atomic coordinates along a path.

In the context of this disclosure machine learning interatomic potential (MLIP) refers to any method that uses machine learning to model and learn the interactions between atoms in materials. In legacy methods (e.g., DFT-based methods) simulation of atomic interactions require complex and computationally intensive calculations. However, the MLIP methods train models with one or more datasets of known atomic configurations and their corresponding/associated energies and forces. Once trained, the trained models can rapidly predict the behavior of atomic systems under different conditions, making it vastly more efficient, computationally and timewise, than legacy methods. In the context of materials science, MLIP methods can enable simulation of larger systems or longer timescales than are feasible with legacy methods. This allows for a more in-depth exploration of material behaviors, properties, and transformations, leading to accelerated discovery and understanding of novel materials and their related phenomena.

Phase 1 machine learning model training subsystem 744 further comprises a machine learning charge density model training subsystem (MLCD model training subsystem) (e.g., machine learning charge density model training subsystem 758) that receives at least one set of charge density values (to visualize, see illustrations of spatial representation 798a, spatial representation 798b, and graphical simulation data view 798c of the shown charge density representations group 798) that is used by the machine learning charge density model training subsystem to train the machine learning charge density model (MLCD model) with the at least one set of charge density values for a specific material configuration received as input.

The set of charge density values that is received as input is a discretized scalar density field model that spans a volume representing an atomic structure, where the charge density values (shown as dense blobs) are scaler values distributed throughout a unit cell shown as a 3D box with many points distributed throughout. Example graphical simulation data view 798c depicts a 3D view of graphical simulation data of charge density in one embodiment. The charge density values may be in the context of electron charge density, hole charge density, nuclear charge density, ionic charge density, or any charged matter as applicable. The GAD application and the MLCD predictive model can be used in the context of any scalar density field.

In the context of this disclosure, a “machine learning charge density model” refers to a machine learning model trained to predict the charge density associated with a specific material configuration. In this context charge density is a discretized scalar density field that spans the volume represented in the associated atomic structure/material configuration and is represented by a set of charge density values. In other words, charge density refers to the distribution of an electric charge over a certain volume or surface. It measures how much electric charge is packed into a given space. In materials science, particularly when discussing atoms and molecules, charge density provides insights into the electron distribution around atomic nuclei. A detailed determination of charge density is crucial, as it directly impacts the material's electronic properties, chemical reactivity, and bonding characteristics. By modeling charge density, an accurate prediction of atomic and/or molecular interactions, bonds, and the global (e.g., gross and macroscopic) properties of materials can be made.

Phase 1 machine learning model training subsystem 744 further comprises a machine learning property predictor model training subsystem (e.g., machine learning property predictor model training subsystem 759). The machine learning property training subsystem receives at least one material property model and trains an internal predictive model on the specific material property/properties selected. The machine learning property model is trained to predict a scalar value of a global property associated with the entire material configuration (including the atomic structure) associated with it. As discussed hereinabove, the global property examples include band gap, electrical conductivity, formation energy, etc., and is not limited to these alone. In the embodiment of FIG. 7B, examples of global properties of materials under training comprise Property A training 761, and Property B training 763 through Property P training 765, where for example property training could pertain to an electrical conductivity or a band gap value or any one of a number of properties of a material.

In some embodiments, the model training subsystem may optionally include a quantum property learning (QPL) model training subsystem such as 755 that is a machine learning model that trains one or more quantum property models to learn other quantum properties (e.g., quantum property Q-prop 757A through quantum property Q-Prop 757M), which may include quantum properties such as bipartite entanglement spectrum, thermal density matrix, ground state energy of a Hamilton operators or functions, spin expectation values, etc. In one embodiment, quantum property learning may be implemented using variational quantum thermalizer (VQT) as is known in the art.

The generative atomistic design application model also comprises an instance of error computing module 7671, which is a part of this active learning framework where model errors and uncertainty estimates are computed at the end of a training phase and/or a training epoch/regime, and a decision is made as to whether or not a newly evaluated structure must be recomputed with DFT using DFT tools 741. If the errors and uncertainties exceed a selected threshold, the structure will be passed back to the DFT subsystem. This feedback mechanism is used to enhance the predictive machine learning models (e.g., models in the form of one or more atomic structures, models in the form of periodic boundary conditions, etc.). Further, this feedback mechanism is used to enhance or confirm any one or more of, corresponding/associated global energy values, corresponding/associated interatomic force models, corresponding/associated charge density values, and/or corresponding global property values.

Following this approach, the machine learning models are trained to sufficient accuracy with fewer expensive DFT computations. To compute predictive model errors, the shown instance of error computing module 7671 compares machine learning interatomic potential model training subsystem outputs (e.g., from MLIP model training subsystem 745), machine learning charge density model outputs (e.g., from machine learning charge density model training subsystem 758), and machine learning property predictor model training outputs (e.g., from machine learning property predictor model training subsystem 759) with their corresponding ground truth values generated by the DFT tools and functions subsystem. In this case, the shown instance of error computing module computes errors by comparing the predicted quantum property values from quantum property learning models with generated quantum property values (e.g., from the phase 0 output dataset generated by operation of quantum tools 743. Once model training converges based on a threshold for error, which is either set by a user/expert or by other means (e.g., self-consistently determined within the active learning cycle), the error is assessed, and if the performance is not satisfactory (e.g., where the error breaches a predetermined error-level threshold), phase 0 is repeated with additional phase 0 input and more data is added to phase 0 output dataset 742. Thereafter, the training processes of Phase 2 are repeated.

In some embodiments, criteria for training convergence may be set even when, after the learning rate is sufficiently low, no further improvement in the error is seen. This process is continued until the computed error, including uncertainty estimates, breaches the selected threshold. One method of determining model error is to use a mean-square error computation and minimize the computed mean-square error between a predicted value from a predictive model and an expected value obtained from the determined ground truth. Typically, model uncertainties may be computed in terms of mean and standard deviation computations, and a chi-squared distribution may be used to compute a goodness value associated with the model uncertainties to compare with a threshold. It is also possible to obtain uncertainty estimates using other statistical methods.

The output of Phase 1 (such as a machine learning model training phase) is various sets of machine learning material models of atomic structures, associated periodic boundary conditions, associated global energy predictive models, associated interatomic force models, associated charge density values, and one or more associated global property value predictive models. These various sets of machine learning material models are then used in other phases to perform various tasks/steps including inference/prediction, relaxation, estimation, refinement, and/or other tasks/steps not listed here.

### Phase 2: Dataset Generation Phase

In some embodiments, such as the embodiment shown in FIG. 7C, the generative atomistic design application model (GAD application) includes Phase 2 in the execution/simulation regime, which is a dataset generation phase that is implemented using prediction subsystem 750. In the dataset generation phase, predictive machine learning is used to predict and/or infer datasets for generative machine learning that follows.

Prediction P subsystem 750 comprises machine learning interatomic potential prediction 775, which comprises a capability to receive one or more atomic structures and one or more corresponding/associated periodic boundary conditions, MLIP predictive models, MLCD predictive models, machine learning proportionality (MLProp) predictive models, and/or any other predictive models included in trained predictive models 751. The MLIP predictive model includes the capability to predict interatomic forces as is shown by interatomic force predictor 777, and further includes the capability to predict global energy values as is shown by global energy predictor 779. In various embodiments, the predictive models may be based on various neural network models such as graph neural networks, convolutional neural networks, transformers, tensor field networks, multi-layer perceptrons (e.g., as shown by multi-layer perceptron 7100 in FIG. 7G), etc.

Prediction subsystem 750 may also comprise machine learning charge density predictor 781 that receives one or more predicted and/or learned structures from Phase 1, and uses machine learning inference to infer or predict corresponding/associated charge density values.

In some embodiments, prediction subsystem 750 may also comprise machine learning property predictor 783 that uses previously trained global property models (or macro property models) that predict values of one or more global properties (e.g., property A prediction 785, property B prediction 787, and property C prediction 789).

The outputs of the prediction subsystem are stored in memory in a data structure such as phase 2 predicted dataset 799, which may be forwarded to other subsystems or subroutines or functions or capabilities in various phases of the GAD application.

In some embodiments, quantum property learning predictive model (e.g., quantum property predictor 791) and/or any model trained in a previous training phases may also be used to predict quantum property values (e.g., quantum property values Q-Property_{A} 793 and quantum property value Q-Property_{B} 795 and quantum property value Q-Property_{J} 797), which may be further used to provide higher fidelity global property values where and when feasible.

### Phase 3: Material Generation Phase

In the context of a generative atomistic design application (a GAD application), “material generation and refinement” is a term used to refer to learning and generation of newer atomic structures (or molecular structures or material configurations). In one embodiment, such as is shown in FIG. 7D, Phase 3 of the GAD application is a material generation phase is implemented using a generative model training subsystem (e.g., generative model training subsystem 703), a portion of which also serves the function of material generation (e.g., as implemented by material generation subsystem 721). In one embodiment, the generative model training subsystem 703 and/or material generation subsystem 721 uses generative machine learning, and is trained to learn and create material configurations (e.g., atomic structures, molecular structures etc.) that may yield new materials with optimized properties. The generative model training subsystem 703 and/or material generation subsystem 721 may be implemented using any known or new generative machine learning models.

In the embodiment shown in FIG. 7D, generative model training subsystem 703 is implemented using an auto encoder (AE) unit or variational auto encoder (VAE) unit. The generative model may be implemented using hybrid auto encoders with diffusion, or hybrid variational auto encoders with diffusion, generative adversarial networks (GANs), or any number of other variations. The generative model is trained to reconstruct the input structure and charge density using mean squared error (MSE). The Kullback-Leibler divergence (e.g., KL divergence) is the final loss term. This causes the model to create a latent space with zero mean and unit variance, which facilitates sampling.

The AE unit or VAE unit in generative model training subsystem 703 may be implemented wholly in software or partially in software and partially in hardware or substantially in hardware that is configurable under software control. The AE/VAE unit trains on a material dataset that may be generated by another phase such as the dataset generation phase. The AE/VAE unit implemented using encoder-decoder architecture comprises material encoder 705, data-structure 715 for storing one or more latent vectors (e.g., latent vector Z1) in an LZ latent space 717, a property predictor and material decoder 707 that generates candidate material configurations such as material configuration 711 (which may be, for example, a candidate periodic atomistic structure in a representation as a unit cell, and/or in a representation involving atomic structures with PBC, molecular structures, etc.) along with the associated charge density values and global property values, which are stored in a data structure.

The material encoder, the property predictor, and the material decoder may have the same or a different number of training epochs. Generally, the material encoder, the property predictor, and the material decoder (e.g., material generator) are trained together in a sequence until a convergence is achieved using an input such as atomic structure 709 and some associated property/properties value. (Note that there may be variations in the exact manner of training between different embodiments, and one single method is not mandated.) Additionally, it is to be appreciated that the atomic structure 709 includes a trained dataset of atomic structures, and that the atomic structure 709A includes a refined dataset of atomic structures.

Any/all of the foregoing predictors may be implemented using a network of predictive multi-layer perceptrons (e.g., multi-layer perceptron 7100) or any other predictive network such as a graph neural network etc., that can perform inferencing and/or make predictions that are portions of or derive from training data. In some cases, a machine learning property predictor can be configured to predict property values using mean squared error methods.

In the material generation portion of Phase 3, the material generation subsystems comprising a previously trained instance of material decoder 707 is provided a latent vector (such as latent vector Z2) as input, and the decoder generates a material configuration (periodic or non-periodic atomic structure, molecular structure etc.) as output. Of note, the generated instance of material configuration 711 may not be stable. Also, as the latent vector Z2 changes, so does the generated material. In one embodiment, a latent vector such as latent vector Z1 or latent vector Z2 in the latent space LZ may be implemented using an array of 64 values. The size “64” of a latent vector may be chosen arbitrarily or may be chosen based on experimentation, and without loss of generality, can be any whole number value that is computationally reasonable and can be used to represent the vast space of material configurations that can be generated. The latent vector Z2 is sampled from the latent space LZ using a selected method (e.g., random sampling, neighborhood sampling, etc.). Latent vector Z2 is passed to the trained decoder, which transforms the latent vector into an atomic structure/material configuration as the case may be. The generated atomic structure/material configuration is then passed to a refinement phase.

Generated candidate material configurations may not be “stable” and may have unbalanced interatomic forces that may render a real-world material with such a material configuration (e.g., atomic structure, molecular structure, etc.) as to be unstable and prone to disintegration/decomposition. Therefore, unstable material configurations-such as non-periodic atomic structures, periodic atomic structures, or molecular structures that are deemed unstable-must be structurally relaxed or discarded so they do not disintegrate/decompose.

### Phase 4: Refinement Phase

Material configurations (periodic or non-periodic atomic structures, and molecular structures, etc.) generated in a material generation phase (e.g., in Phase 3) may have very high and/or unbalanced interatomic forces and/or global energy values, which may render such material configurations unstable in the real world. To obtain stable materials with desired properties, such unstable material configurations must be relaxed, and this relaxation is done by refinement subsystem 756. As shown, refinement subsystem 756 includes an instance of a relaxer 723 that in turn comprises a machine learning interatomic potential predictor (e.g., MLIP predictor 725). The relaxer implements any one or more types of relaxation processes such as simulated annealing or other mechanisms that relocates the atoms slowly to simulate time-varying interatomic forces. This mechanism could be, in principle, any local or global optimization algorithm, gradient based or gradient-free. Typically, in various embodiments of these applications, they would be local, gradient-based optimization algorithms (e.g., the Broyden-Fletcher-Goldfarb-Shanno algorithm (BFGS algorithm), the fast inertial relaxation engine (FIRE), conjugate-gradient method, and/or quasi-Newton methods, etc.).

A machine learning interatomic potential predictive model may be used to quickly determine the values of the interatomic forces on relaxed material configurations during the relation process. Further, a machine learning charge density predictor such as MLCD predictor 729, a machine learning property predictor such as MLProp predictor 731, along with a quantum property predictor such as qProp predictor 733 may be used, singly or in combination, to determine the charge density profile, the global property values, and the quantum property values of a relaxed material configuration. This may cause the relaxer to adjust the position of one or more atoms in a generated structure to further reduce the interatomic forces on the atoms in the structure until a stable material configuration is obtained.

In one embodiment, the atoms and/or molecules, when initially placed in a simulated environment, may not be in their lowest energy position. In operation, relaxer 723 adjusts the positions of the one or more atoms in response to the forces predicted by MLIP predictor 725. The process of relaxing a structure (e.g., structure relaxer 790) allows the atoms and/or molecules to settle into their locally lowest energy configurations, which often correspond to how they would naturally arrange themselves in reality in the physical world. The processes of FIG. 7D may be repeated by feeding the relaxed structure to MLIP predictor 725 and continuing the process of relaxation until the maximum force on any atom is below a stable maximum force threshold value, which may be set by a user/expert. For example, an acceptable stable maximum force threshold value Fmax may be in the range FX £ Fmax £ FY for a given material under consideration.

Machine learning charge density predictor 781 is used to predict the charge density of a relaxed configuration of an atomic structure at any point during the simulation. A machine learning property predictor (e.g., MLProp predictor 783) is used to predict one or more property value predictions (e.g., property A prediction 785, property B prediction 787, property C prediction 789) associated with the relaxed configuration of atomic structure 709. The extensive use of machine learning based inference/prediction significantly reduces the need to use DFT-based calculations, which can be one or more orders of magnitude computationally expensive.

After the generation of a material configuration, the goodness estimate for the result is computed in terms of a model error and/or model uncertainty estimates. Specifically, in Phase 4, model error is computed as mean squared error taken as a square of the difference between the phase 4 predicted values after the refinement steps and the expected values from the raw outputs (e.g., atomic structures, charge density values, property values, etc.) of the generative model.

To compute predictive model errors, the shown instance of error computing module 7672 compares machine learning charge density model values (e.g., from MLCD predictor 729, ML property model values (e.g., from MLProp predictor 731),), and quantum property values (e.g., from Qprop predictor 733) with their corresponding ground truth values generated by the DFT tools and functions subsystem.

A desired threshold is chosen by simulation/experimentation within a reasonable range, and if a computed uncertainty and/or model error for a candidate material configuration (periodic or non-periodic atomic or molecular structures) is above the chosen threshold, then the candidate unit cell/atomic structure is passed back to the DFT subsystem in phase 0 to recompute accurate energy values, interatomic forces, charge density, and one or more property values accurately. The computed energy (e.g., global energy value), interatomic forces, charge density, and the one or more property values are used to further train the MLIP models, the MLCD models, and the machine learning property models in Phase 1. If the model error for a candidate material configuration (e.g., atomic structure) is below the chosen threshold, then the model is promoted to be included into refined output dataset 768.

### Phase 5: Material Selection and Validation Phase

In the embodiment of FIG. 7E, generative atomistic design application 724 (GAD application) and refined output dataset 769 hold material configurations and/or atomic structures and their corresponding/associated interatomic potential, charge density, and generated energy structures generated by material generation subsystem 721 in the material generation phase (e.g., in Phase 3), which had been subsequently refined by refinement subsystem 756. Refined output dataset 769 is queried to determine the best M candidate material configurations (e.g., the shown top N configurations 737) that are predicted to exhibit the property values in a desired range. The best N candidate material configurations can be based on a goodness factor for comparing any number of candidate material configurations. In some embodiments, <n> is an integer provided by a user/expert. For example, in one embodiment, if materials that minimize formation energy are designed, where n might be desired to be 20, 20 materials may be drawn from the phase 3 dataset based on whichever of those 20 materials have the lowest predicted formation energy. These selected materials are passed to DFT tools 741 and/or to quantum models for validation (e.g., by way of generating their formation energy or by way of confirming material properties). Now, with an understanding that there is a limited number of best candidates to be selected and promoted to undergo accurate simulation using DFT tools, it can be seen that this technique is many orders of magnitude more efficient than alternative approaches (e.g., where a nearly unlimited number of candidates of unknown promise are subjected to further simulation).

The set of atomic structures (e.g., unit cells, periodic atomic structures, material configurations, etc.) that are considered to be best for one or more macro properties, are placed in the final output dataset 762 along with their associated charge density values, interatomic forces, global energy values, and global properties. Characteristics of the foregoing materials are known to be accurate, at least in that they have computed to the accuracy level of DFT tools 741. Further, foregoing quantum tools 743 are used to calculate and/or validate various quantum properties of selected materials.

In some embodiments, one or more atomic structures/material configurations in final output dataset 762 may be used to synthesize or fabricate a physical material (e.g., via synthesis and fabrication process 738).

FIG. 7G depicts a multi-layer perceptron. Any known technique can be used to establish and assign values to individual neurons of a perceptron. As used herein a multilayer perceptron (MLP) refers to a feedforward artificial neural network consisting of interconnected neurons, An MLP can implement nonlinear activation functions. Example embodiments are organized into at least three layers (e.g., as shown in FIG. 7G).

FIG. 7H is a block diagram of an instance of a computer system 7H00 suitable for implementing embodiments of the present disclosure. Computer system 7H00 includes bus 782 or other communication mechanism for communicating information. The bus interconnects subsystems and devices such as a central processing unit (CPU), or a multi-core CPU (e.g., data processor 786), a system memory (e.g., main memory 774, or an area of random access memory (RAM) implemented in any technology), a non-volatile memory in any technology such as FLASH memory or phase change memory or a non-volatile storage device or non-volatile storage area (e.g., read-only memory 776), a graphics memory (e.g., graphics memory 778), internal storage device 780 or external storage device 7108 (e.g., magnetic or optical or photonic or quantum electronic or ferroelectric or semiconductor or phase change memory), data interface 784, and communications interface 788 (e.g., PHY, MAC, Ethernet interface, modem, etc.). The aforementioned components are shown within a processing element partition, however other partitions are possible.

Computer system 7H00 further comprises display 770 (e.g., CRT or LCD or OLED or 3D holographic display), various input devices 772 (e.g., keyboard, cursor control, camera, light pen, etc.), and an external data repository 7101.

According to an embodiment of the disclosure, computer system 7H00 performs specific operations by data processor 786 executing one or more sequences of one or more program instructions contained in a memory. Such instructions (e.g., program instructions 71171, program instructions 71172, program instructions 71173, and/or program instructions 71174, etc.) can be contained in or can be read into a storage location or memory from any computer readable/usable storage medium such as a static storage device or a hard drive. The sequences can be organized to be accessed by one or more processing entities configured to execute a single process or configured to execute multiple concurrent processes/tasks/threads to perform work. A processing entity can be hardware-based (e.g., involving one or more cores of homogenous or heterogenous processing elements) or software-based, and/or can be formed using a combination of hardware and software that implements logic and/or can carry out computations and/or processing steps using one or more processes and/or one or more tasks and/or one or more threads or any combination thereof.

According to an embodiment of the disclosure, computer system 7H00 performs specific networking operations using one or more instances of communications interface 788. Instances of communications interface 788 may comprise one or more networking ports that are configurable (e.g., pertaining to speed, protocol, physical layer characteristics, media access characteristics, etc.), and any particular instance of communications interface 788 or port thereto can be configured differently from any other particular instance. Portions of a communication protocol can be carried out in whole or in part by any instance of communications interface 788, and data (e.g., packets, data structures, bit fields, etc.) can be positioned in storage locations within communications interface 788, or within system memory, and such data can be accessed (e.g., using random access addressing, or using direct memory access DMA, etc.) by devices such as data processor 786.

Communications link 7110 can be configured to transmit (e.g., send, receive, signal, etc.) any type of communications packets (e.g., communication packet 7111₁, . . . , communication packet 7111_{N}) comprising any organization of data items. The data items can comprise payload data area 7115, destination address 7114 (e.g., a destination IP address), source address 7113 (e.g., a source IP address), and can include various encodings or formatting of bit fields to populate packet characteristics 7112. In some cases, the packet characteristics include a version identifier, a packet or payload length, a traffic class, a flow label, etc. In some cases, payload data area 7115 comprises a data structure that is encoded and/or formatted to fit into byte or word boundaries of the packet.

In some embodiments, hard-wired circuitry (or hardware) may be used in place of or in combination with software instructions to implement aspects of the disclosure. Thus, embodiments of the disclosure are not limited to any specific combination of hardware circuitry and/or software (program instructions). In embodiments, the term “logic” shall mean any combination of software or hardware including but not limited to a microcode implementation or a programmable gate array (PGA/FPGA) implementation that are used to implement all or part of the disclosure.

The term “computer readable medium” or “computer usable medium” as used herein refers to any medium that participates in providing instructions to data processor 786 for execution. Such a medium may take many forms, including but not limited to, non-volatile media and volatile media. Non-volatile media includes, for example, optical or magnetic disks such as hard drives or disk drives or tape drives. Volatile media includes dynamic or static memory such as RAM or a register file or a sequential memory or even a phase-change memory.

Common forms of computer readable media include, for example, floppy disk, flexible disk, hard disk, magnetic tape, or any other magnetic medium; CD-ROM or any other optical medium; punch cards, paper tape, or any other physical medium with patterns of holes or micro-dots of phase altered materials; RAM, PROM, EPROM, FLASH-EPROM, phase-change memory or any other memory chip or cartridge; or any other non-transitory computer readable medium. Such data can be stored, for example, in any form of external data repository 7101, which in turn can be formatted into any one or more storage areas, and which can comprise parameterized storage 7102 accessible by a key (e.g., filename, table name, block address, offset address, etc.).

In the context of this disclosure a database comprising material classes and their properties, structures, and functional models of density functional theory-based implementations and data, experimental data from materials testing and validation, quantum properties data (e.g., quantum states, Hamiltonian operators or functions, correlation functions, etc.), quantum property learning functions and models, (e.g., quantum machine learning data, functions, and models), and various other simulation parameters may be stored on external data repository 7101 and/or a portion of the database may be stored on external storage device 7108. Further, a portion of the database may be stored on parameterized storage 7102 and furthermore, a portion of the database may be stored in or on database server 7103, and/or in or on any one or more external storage devices. Strictly as examples, any one or more of material properties 7104, DFT database 7105, expert data 7106, and/or Qt modeling language (QPML) database 7107 can be stored in parameterized storage.

Execution of the sequences of instructions to practice certain embodiments of the disclosure are performed by a single instance of computer system 7H00. According to certain embodiments of the disclosure, two or more instances of computer system 7H00 coupled by communications link 7110 (e.g., LAN, public switched telephone network, or wireless network) may perform the sequence of instructions required to practice embodiments of the disclosure using two or more instances of components of computer system 7H00.

Computer system 7H00 may transmit and receive messages such as data and/or instructions organized into a data structure (e.g., communications packets). The data structure can include program instructions (e.g., application code 7109), communicated through communications link 7110 and communications interface 788. Received program instructions may be executed by data processor 786 as it is received and/or stored in the shown storage device or in or upon any other non-volatile storage for later execution. Computer system in 7H00 may communicate through a data interface 784 to a database server 7103 on an external data repository 7101. Data items in a database may be accessed using a primary key (e.g., a relational database primary key) or the data items may be accessed using simple functional APIs where a key may or may not be used/needed.

Processing element partition 768 is merely one sample partition. Other partitions can include multiple data processors, and/or multiple communications interfaces, and/or multiple storage devices, etc. within a partition. For example, a partition can bound by a multicore processor (e.g., possibly including embedded or collocated memory), or a partition can bound a computing cluster having a plurality of computing elements, any of which computing elements are connected directly or indirectly to a communications link. A first partition can be configured to communicate to a second partition. A particular first partition and particular second partition can be congruent (e.g., in a processing element array) or can be different (e.g., comprising disjoint sets of components).

A module as used herein can be implemented using any mix of any portions of the system memory and any extent of hard-wired circuitry including hard-wired circuitry embodied as a data processor 786. Some embodiments include one or more special-purpose hardware components (e.g., power control, logic, sensors, transducers, etc.). Some embodiments of a module include instructions that are stored in a memory for execution so as to facilitate operational and/or performance characteristics pertaining to the present disclosure. A module may include one or more state machines and/or combinational logic circuits used to implement or facilitate the operational and/or performance characteristics pertaining to the present disclosure.

Various implementations of database server 7103 comprise storage media organized to hold a series of records or files such that individual records or files are accessed using a name or key (e.g., a primary key or a combination of keys and/or query clauses). Such files or records can be organized into one or more data structures (e.g., data structures used to implement or facilitate aspects of the present disclosure. Such files, records, or data structures can be brought into and/or stored in volatile or non-volatile memory. More specifically, the occurrence and organization of the foregoing files, records, and data structures improve the way that the computer stores and retrieves data in memory, for example, to improve the way data is accessed when the computer is performing operations pertaining to the present disclosure, and/or for improving the way data is manipulated when performing computerized operations pertaining to the present disclosure.

FIG. 7I depicts a multi-cloud computing environment 7100. As shown, a first computing cloud (shown as cloud1) is interconnected with a second computing cloud (shown as cloud2). Any known inter-cloud communication technique or combination of inter-cloud communication techniques can be used to facilitate bi-directional data communication that may be demanded when interfacing GAD application simulation and I/O tasks 7118 with inferencing tasks 7119.

In some cases, cloud1 hosts multiple different tenants in a manner such that data corresponding to each of the multiple different tenants may be segregated based on physical or logical boundaries. For example, a first tenant may run a first instance of a GAD application in a first computing platform (e.g., computing platform 718_{T1}), whereas a second tenant may run a second instance of a GAD application in a second computing platform (e.g., computing platform 718_{T2}). In this manner, first tenant-specific data can be securely segregated from any other tenant's tenant-specific data.

In this example partitioning of FIG. 7I, the computing facilities of cloud1 are substantially dedicated to performing GAD application simulation and I/O tasks, whereas the computing facilities of cloud2 are substantially dedicated to performing inferencing. More particularly, the computing facilities of cloud1 perform simulation tasks and then orchestrate communication to/from cloud2 such that the computing facilities of cloud2 can be substantially dedicated to performing inferencing. To do so in this example partitioning, modules situated on cloud1 send training data 7120 to modules of cloud2, and modules of cloud2 send inferencing data 7121 to modules of cloud1.

In the example embodiment as is shown in 71, the modules of cloud2 are configured particularly so as to be able to perform inferencing tasks using any number of AI supercomputer cores (e.g., AI supercomputer cores 7122₁, AI supercomputer cores 7122₂, AI supercomputer cores, supercomputer cores 7122_{N}), individual ones of, or combinations of the foregoing AI supercomputer cores have core-dedicated local memory. Any individual ones of, or combinations of the AI supercomputer cores can access any of a plurality of decoupled memories (e.g., decoupled memory 7124₁, decoupled memory 7124₂, decoupled memory 7124_{N}). Moreover, individual ones of the AI supercomputer cores can be aggregated into tenant-specific core groups and configured dynamically so as to comport with then-current inferencing and other then-current computing tasks as demanded by particular tenant's operation of that tenant's GAD application. To accommodate such aggregation and dynamic configuration, a core aggregator 7125, possibly in coordination with a corresponding instance of parallelizer 7126 can allocate, aggregate and train a tenant-specific set of the foregoing AI supercomputer cores and/or populate the foregoing decoupled memories with tenant-specific data. Communication of configuration data, training data and inferencing data to and from individual ones of the tenant-specific AI supercomputer cores as well as communication of configuration data, training data and inferencing data to and from individual ones of the tenant-specific set of the foregoing AI supercomputer cores and the decoupled memories can be accomplished by inter-module communications over backplane 7123. Any known techniques for backplane communications in combination with any known technologies for backplane communications can be used. For example, a backplane might be implemented using a high-speed, low-latency fabric based on optical transceivers.

It should be noted that the foregoing AI supercomputer cores can include many millions of perceptrons. Moreover, it should be noted that the foregoing AI supercomputer cores can be configured to accept extremely long training sequences (e.g., tens of thousands of parameters, or hundreds of thousands of parameters, and longer).

FIG. 7J shows a use case 7J00 depicting how one or more human users/experts may interact with components of a multi-platform computing environment. The human interactions with the particular shown computing platforms are being presented merely as examples of how human-generated and/or human-selected information can be used to drive an AI supercomputer. More particularly, the figure is being presented to illustrate how humans may operate respective computing platforms (e.g., computing platform 718_{T3}, and 718_{T9}) so as to prepare data (e.g., AI entity inputs 7140) that is in turn provided to an AI supercomputer.

In this particular use case, the foregoing respective computing platforms are configured to interact with users and/or experts through various user interfaces (e.g., graphical user interfaces, text user interfaces, command line interfaces, etc.) that are purpose-designed to support particular types of interactions. Strictly for purposes of illustration, two types of purpose-specific computing platforms are now briefly described.

Material Specification Platform: A first computing platform 718_{T3} is purpose-designed to support receiving and checking human-generated data (e.g., material specifications). This is depicted by provision of human-determined sets of selected inputs 713 to computing platform 718_{T3}. The human-selected inputs are subjected to (1) semantic checks 7130 as well as (2) completeness checks 7132 over the human-selected inputs. As shown, the foregoing human-determined (e.g., user1-determined) specification of selected inputs 713 may include material requests 704, property requests 706 and possibly priority assignments 7128 that are used in downstream processing, for example to indicated preferences when limitations of the computing equipment or configuration are to be considered. Additionally or alternatively, there may be human-selected provision of experimental learnings 708_{SELECTED}, which selections may include or be influenced by any sorts of measured data such as measurements deriving from human operation of metrology equipment.

In some situations, the foregoing human-determined specification (e.g., values, formats, etc.) of selected inputs 713 may derive, in part, from computerized tools. It is at the choice of the user/expert to decide what specifications or values to provide to computing platform 71813, and it is at the choice of the user/expert to decide in what form or format to provide. Furthermore, it is at the choice of the user/expert to decide how to select and/or modify certain feedback data 7144 that may be produced by or derived from the results of downstream processing. In some cases feedback data 7144 is of a nature that is most applicable for a user (e.g., user user1, user user2) to consider. In other cases, feedback data 7144 is of a nature that is most applicable for a practitioner to consider.

Prompt Engineering Platform: A second computing platform 718_{T9} is purpose-designed to support human-driven prompt engineering activities. This is depicted by provision of human-determined prompt data 7136 to prompt engineering module 7134 of computing platform 718_{T9}. In addition to processing of human-determined prompt data 7136, prompt engineering module 7134 can further accept human-curated instances of additional prompt data 7136_{ADDITIONAL}, which may derive, in part, from downstream processing (e.g., deriving from the results of processing AI entity inputs tasks 7140 through any number of AI supercomputer cores 7122_{N}).

As is known in the art, configuration of an AI entity, including establishment and biasing of neurons, and including establishment and weighting of inter-neuron connections, can be carried out in a human-supervised manner where a human user/expert specifies the foregoing weights and bias parameters. Alternatively, or in some cases additionally, configuration of an AI entity may be carried out in a semi-supervised manner where a training set may be specified and the foregoing weights and bias parameters are derived from evaluation of the training set. In any case, the AI entity outputs include a human-generated portion 7142 in combination with an AI-generated portion 7138.

Now, returning to the discussion of prompt engineering activities, it is known in the art that even the largest AI supercomputers have structural limitations (e.g., number of parameters) that imply other limitations (e.g., number of tokens in a prompt). Accordingly, a practitioner participates in the overall flow in a manner that facilitates generation of a prompt that is both effective in providing a meaningful and effective prompt to the AI entity while at the same time comporting to AI supercomputer limitations. To this end, revised material requests 704_{REVISED} may be considered, and/or revised property requests 706_{REVISED} may be considered when forming a meaningful and effective prompt to the AI entity.

Based on the foregoing, it is to be understood that a user enables an AI system through user input. As has been discussed, user may contribute their input in the form of dictating material classes and properties (such as in the initial dataset). In one embodiment, the Ai system may use intent recognition in identifying the user's goals and/or inputs, which in turn may guide subsequent actions. Further, the inputs provided may additionally include contextual understanding for the AI system to ensure that the AI system appreciates the ongoing analysis (such as referencing prior inputs and acknowledging user preferences). In cases where additional information is required, the AI system may retrieve data from various sources and machine learning models (which, again, may be trained on user dictated material classes and properties).

In one embodiment, it is to be appreciated that the core of the AI's functionality may reside in processing and decision-making, which again, is based initially on user dictated input. Further, many of the outputs from the AI system may be presented to the user for continued input through the process. In this manner, the overall system disclosed herein may benefit from both user dictated input and AI processing power, where the AI system may otherwise function as a digital laboratory of testing atomic structures.

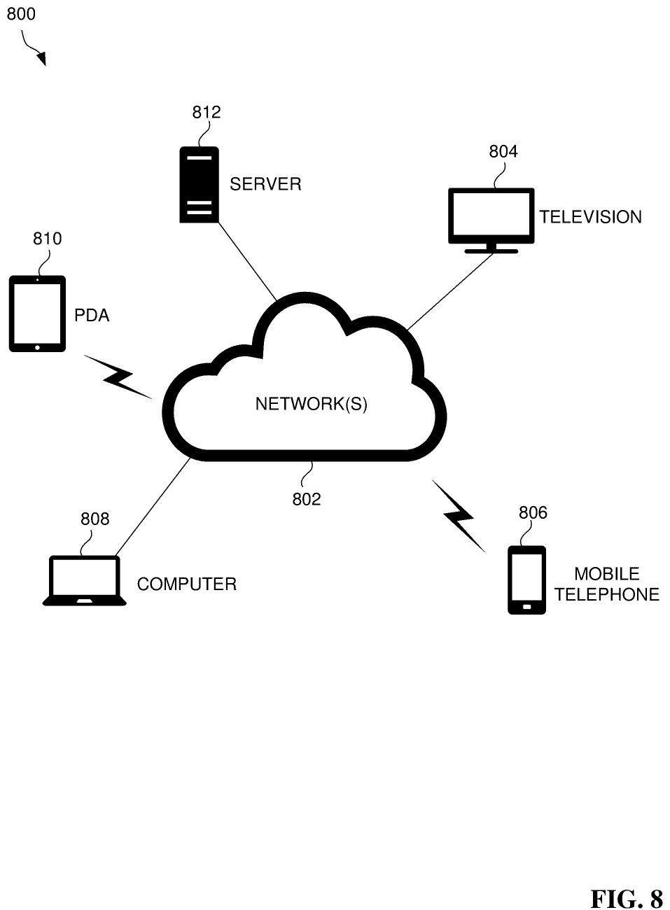

FIG. 8 illustrates a network architecture 800, in accordance with one possible embodiment. As shown, at least one network 802 is provided. In the context of the present network architecture 800, the network 802 may take any form including, but not limited to a telecommunications network, a local area network (LAN), a wireless network, a wide area network (WAN) such as the Internet, peer-to-peer network, cable network, etc. While only one network is shown, it should be understood that two or more similar or different networks 802 may be provided.

Coupled to the network 802 is a plurality of devices. For example, a server computer 812 and an end user computer 808 may be coupled to the network 802 for communication purposes. Such end user computer 808 may include a desktop computer, lap-top computer, and/or any other type of logic. Still yet, various other devices may be coupled to the network 802 including a personal digital assistant (PDA) device 810, a mobile phone device 806, a television 804, etc.

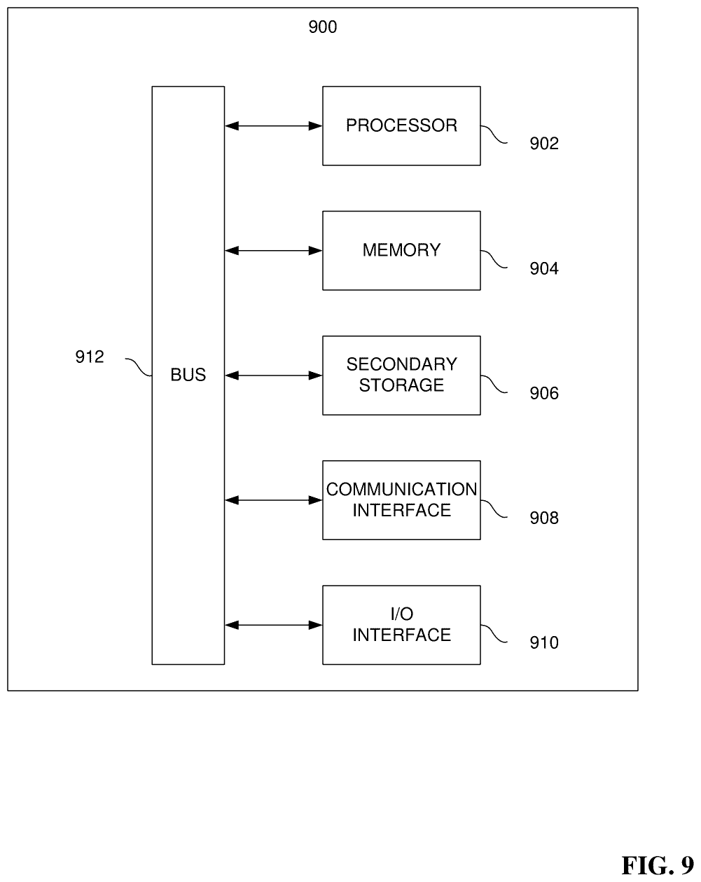

FIG. 9 illustrates an exemplary system 900, in accordance with one embodiment. As an option, the system 900 may be implemented in the context of any of the devices of the network architecture 800 of FIG. 8. Of course, the system 900 may be implemented in any desired environment.

As shown, a system 900 is provided including at least one central processor 902 which is connected to a communication bus 912. The system 900 also includes main memory 904 [e.g. random access memory (RAM), etc.]. The system 900 also includes a graphics processor 908 and a display 910.

The system 900 may also include a secondary storage 906. The secondary storage 906 includes, for example, a hard disk drive and/or a removable storage drive, representing a floppy disk drive, a magnetic tape drive, a compact disk drive, etc. The removable storage drive reads from and/or writes to a removable storage unit in a well known manner.

Computer programs, or computer control logic algorithms, may be stored in the main memory 904, the secondary storage 906, and/or any other memory, for that matter. Such computer programs, when executed, enable the system 900 to perform various functions (as set forth above, for example). Memory 904, storage 906 and/or any other storage are possible examples of non-transitory computer-readable media. It is noted that the techniques described herein, in an aspect, are embodied in executable instructions stored in a computer readable medium for use by or in connection with an instruction execution machine, apparatus, or device, such as a computer-based or processor-containing machine, apparatus, or device. It will be appreciated by those skilled in the art that for some embodiments, other types of computer readable media are included which may store data that is accessible by a computer, such as magnetic cassettes, flash memory cards, digital video disks, Bernoulli cartridges, random access memory (RAM), read-only memory (ROM), and the like.

As used here, a computer-readable medium includes one or more of any suitable media for storing the executable instructions of a computer program such that the instruction execution machine, system, apparatus, or device may read (or fetch) the instructions from the computer readable medium and execute the instructions for carrying out the described methods. Suitable storage formats include one or more of an electronic, magnetic, optical, and electromagnetic format. A non-exhaustive list of conventional exemplary computer readable medium includes: a portable computer diskette; a RAM; a ROM; an erasable programmable read only memory (EPROM or flash memory); optical storage devices, including a portable compact disc (CD), a portable digital video disc (DVD), a high definition DVD (HD-DVD™), a BLU-RAY disc; and the like.

It should be understood that the arrangement of components illustrated in the Figures described are exemplary and that other arrangements are possible. It should also be understood that the various system components (and means) defined by the claims, described below, and illustrated in the various block diagrams represent logical components in some systems configured according to the subject matter disclosed herein.

For example, one or more of these system components (and means) may be realized, in whole or in part, by at least some of the components illustrated in the arrangements illustrated in the described Figures. In addition, while at least one of these components are implemented at least partially as an electronic hardware component, and therefore constitutes a machine, the other components may be implemented in software that when included in an execution environment constitutes a machine, hardware, or a combination of software and hardware.

More particularly, at least one component defined by the claims is implemented at least partially as an electronic hardware component, such as an instruction execution machine (e.g., a processor-based or processor-containing machine) and/or as specialized circuits or circuitry (e.g., discreet logic gates interconnected to perform a specialized function). Other components may be implemented in software, hardware, or a combination of software and hardware. Moreover, some or all of these other components may be combined, some may be omitted altogether, and additional components may be added while still achieving the functionality described herein. Thus, the subject matter described herein may be embodied in many different variations, and all such variations are contemplated to be within the scope of what is claimed.

In the description above, the subject matter is described with reference to acts and symbolic representations of operations that are performed by one or more devices, unless indicated otherwise. As such, it will be understood that such acts and operations, which are at times referred to as being computer-executed, include the manipulation by the processor of data in a structured form. This manipulation transforms the data or maintains it at locations in the memory system of the computer, which reconfigures or otherwise alters the operation of the device in a manner well understood by those skilled in the art. The data is maintained at physical locations of the memory as data structures that have particular properties defined by the format of the data. However, while the subject matter is being described in the foregoing context, it is not meant to be limiting as those of skill in the art will appreciate that various of the acts and operations described hereinafter may also be implemented in hardware.

To facilitate an understanding of the subject matter described herein, many aspects are described in terms of sequences of actions. At least one of these aspects defined by the claims is performed by an electronic hardware component. For example, it will be recognized that the various actions may be performed by specialized circuits or circuitry, by program instructions being executed by one or more processors, or by a combination of both. The description herein of any sequence of actions is not intended to imply that the specific order described for performing that sequence must be followed. All methods described herein may be performed in any suitable order unless otherwise indicated herein or otherwise clearly contradicted by context.

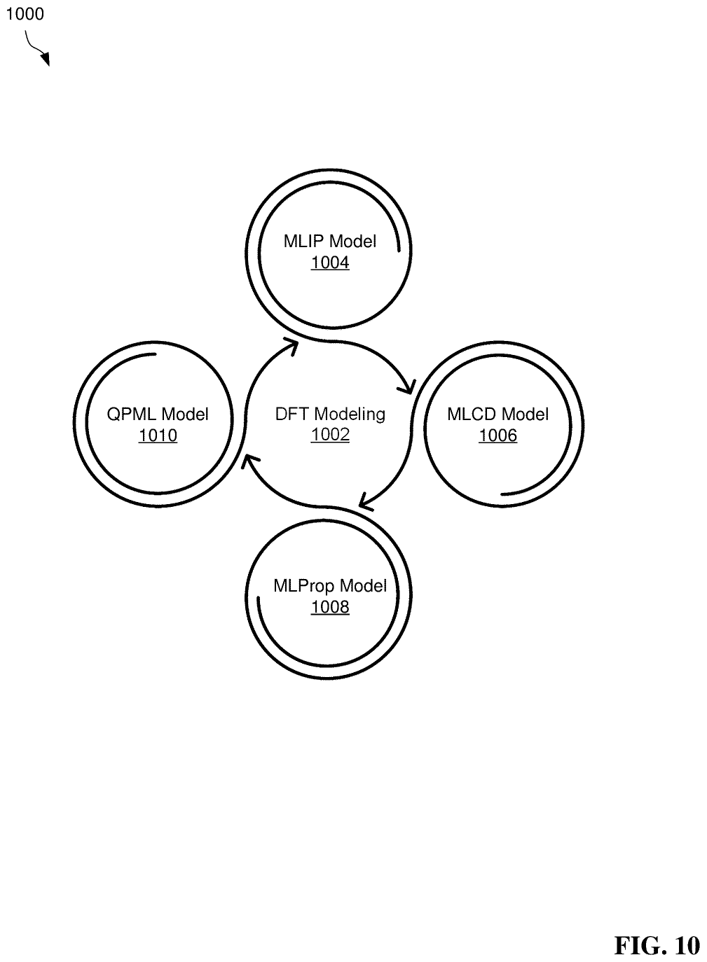

FIG. 10 illustrates a system 1000 for active learning via constant model reevaluation and restructuring, in accordance with one embodiment. As an option, the system 1000 may be implemented in the context of any one or more of the embodiments set forth in any previous and/or subsequent figure(s) and/or description thereof. Of course, however, the system 1000 may be implemented in the context of any desired environment. Further, the aforementioned definitions may equally apply to the description below.

As shown, the system 1000 may show a DFT modeling 1002 approach that relies on many subcomponents. For example, DFT modeling 1002 may rely on a MLIP model 1004, a MLCD model 1006, a MLProp model 1008, and/or a QPML model 1010. It is to be appreciated that the MLIP model 1004, the MLCD model 1006, the MLProp model 1008, and/or the QPML model 1010 may be configured in a serial or parallel manner with respect to the DFT modeling 1002. Thus, in one embodiment, a dataset from DFT may be sent to each of the MLIP model 1004, the MLCD model 1006, the MLProp model 1008, and/or the QPML model 1010 concurrently, or may be sent to a first model, which in turn, may then provide the output to the next model.

In one embodiment, as discussed previously, the GAD application model may compute uncertainty estimates and error calculations at the end of a training phase, and a decision may be made as to whether or not a newly evaluated structure must be recomputed with DFT. As such, if the errors and uncertainties exceed a selected threshold, the structure may be passed back to the DFT subsystem. This feedback mechanism may be used to enhance the predictive machine learning models (e.g., models in the form of one or more atomic structures, models in the form of periodic boundary conditions, etc.). Further, this feedback mechanism may also be used to enhance or confirm any one or more corresponding/associated global energy values, corresponding/associated interatomic force models, corresponding/associated charge density values, and/or corresponding global property values.

In a related embodiment, using the at least one processor, a new material may be predicted with desired properties using at least two machine learning models as shown within the context of the system 1000, where the at least two machine learning models are trained based on the desired properties, analyzing the prediction of the new material via the at least two machine learning models, and outputting the prediction once the prediction exceeds a predetermined threshold associated with the at least two machine learning models.

In another embodiment, an active learning cycle may be created, using at least two machine learning models of the system 1000, by generating training data based on property data, iteratively reprocessing the property data and the training data until a predetermined threshold is met, where the predetermined threshold includes a set of characteristics, and once the predetermined threshold is met, generating a predictive model, generating a machine learning model using the predictive model, and applying the machine learning model to predict a new material having the characteristics. As such, the DFT modeling 1002 may be used in an iterative manner to create an active learning environment, where the machine learning models may be used iteratively to train, generate, and refine novel materials.

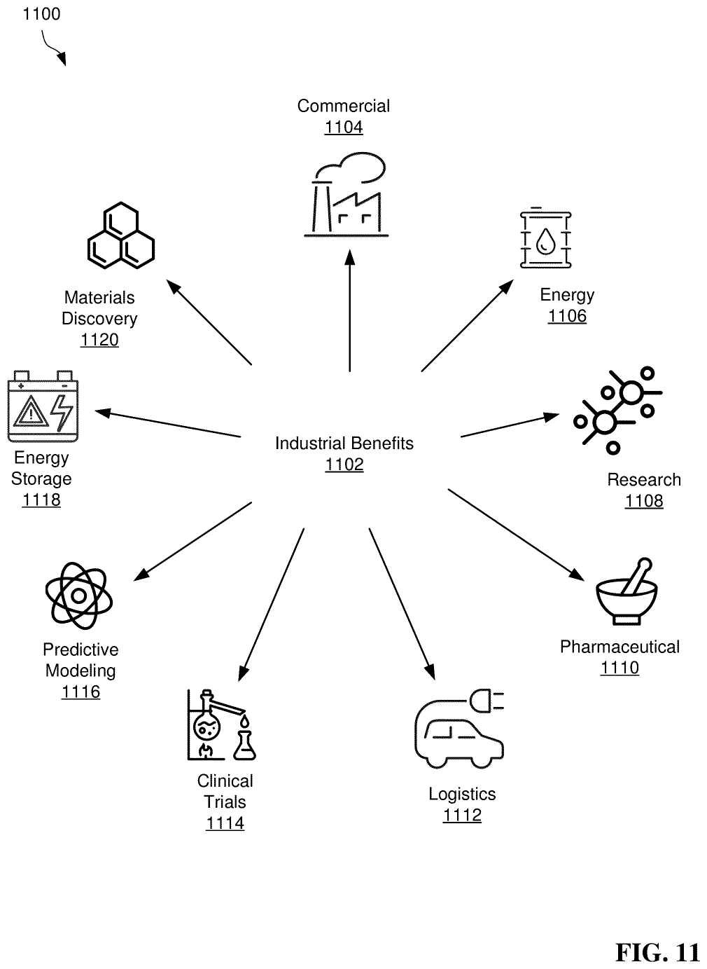

FIG. 11 illustrates a collection 1100 of possible industrial benefits of novel atomic structure discovery through machine-learned modeling, in accordance with one embodiment. As an option, the collection 1100 may be implemented in the context of any one or more of the embodiments set forth in any previous and/or subsequent figure(s) and/or description thereof. Of course, however, the collection 1100 may be implemented in the context of any desired environment. Further, the aforementioned definitions may equally apply to the description below.

As shown, industrial benefits 1102 may include commercial 1104, energy 1106, research 1108, pharmaceutical 1110, logistics 1112, clinic trials 1114, predictive modeling 1116, energy storage 1118, materials discovery 1120, etc. It is to be appreciated that any industrial benefit 1102 shown is merely exemplary and should not be limiting to the disclosure herein in any manner.

Further, the collection 1100 is intended to represent the wide applicability of the present disclosure to a variety of industries. Materials, at a global context level, relates to every human throughout the world. As such, generation of novel materials may affect all industries worldwide.

Within the context of synthesis, fabrication may involve the creation of the desired material. For example, fabrication may include shaping, assembly, and/or construction of materials into functional devices or structures. While synthesis may focus on creating the material itself, fabrication may involve using that material to build something else (i.e. a device, an end product, etc.). For example, fabrication processes may range from depositing thin films for electronics, and/or carving nanostructures using techniques like lithography, to assembling larger components for mechanical systems. Whether constructing a new type of solar cell, developing a nanoscale sensor, or building a robust mechanical part, fabrication may turn raw materials into functional components that may be integrated into real-world applications.

In various embodiments, periodic boundary conditions (PBC) may include a mathematical tool used in simulations where a system (like a group of atoms) may be imagined to repeat indefinitely in all directions. By using PBC, a small segment may be simulated while effectively mimicking the behavior of a much larger, infinite system. This technique may be especially useful in reducing computational costs while still capturing the essence of large-scale phenomena.

In another embodiment, quantum mechanics (QM) may include behaviors and properties of matter and energy at their smallest scales, typically at the level of atoms and subatomic particles.

In still another embodiment, density functional theory (DFT) may be used to investigate the electronic properties of multi-body systems (such as atoms, molecules, and/or solids). For example, rather than attempt to track each electron's movement, which can be very complex, a DFT process may focus on overall electron density-that is, the manner in which electrons may be distributed in space, and in so doing, DFT may simplify the problem and make it feasible to predict how atoms and molecules will behave in different conditions.

In yet another embodiment, machine learning (ML) may be a subset of artificial intelligence that may involve teaching computers to identify patterns and make decisions from data, without being explicitly programmed for specific tasks. As such, in the context of materials science, ML may be employed to predict material properties, behaviors, and/or even to design new materials. By analyzing precomputed data-from atomic structures to system dynamics-ML algorithms may be able to efficiently identify relationships that might otherwise be too complex to discern using traditional methods.

In one embodiment, generative machine learning (generative ML) may be a function of artificial intelligence, where algorithms may be trained to generate new data resembling a given set of inputs. Thus, generative models may be used to create new designs or configurations of materials based on patterns and structures they have learned from existing data. As such, rather than just predicting properties of known materials, generative machine learning models may propose entirely new materials or suggest modifications to existing materials optimized for specific desired properties.

In one embodiment, predictive machine learning (Predictive ML) may train algorithms to make predictions about future and/or unseen data based on patterns identified from historical or known data, where the primary goal may be to output a specific value or classification based on input features. For example, predictive ML may be used to forecast the conductivity, strength, and/or melting point of a material based on its atomic structure.

In one embodiment, active learning (AL) may selectively query users (or a data source) with an algorithm in order to obtain labels for specific data points the algorithm may consider most informative. Rather than passively receiving a pre-labeled dataset, an active learning model may actively seek examples it believes may improve performance the most.

In one embodiment, Bayesian Optimization (BO) may find a maximum (or minimum) value of a function where direct evaluations of the function may be costly or limited. In practice, Bayesian Optimization may use probabilistic models (which may often be Gaussian processes) to predict the outcome of untested inputs and, importantly, the uncertainty associated with those predictions. Additionally, the optimization method may then balance exploring regions of high uncertainty against exploiting regions of expected high performance. For example, Bayesian Optimization may be instrumental when exploring a vast space of potential material compositions or synthesis conditions, where it is often impractical to test every possibility. Bayesian Optimization may also aid in pinpointing the most promising candidate materials by strategically choosing which experiments or simulations to conduct next, based on both previous results and the predicted outcomes. As such, by iteratively refining its understanding, Bayesian Optimization may efficiently direct experimental and computational efforts, and thus may accelerate the discovery of optimal atomic structures. In some aspects, the techniques described herein relate to a method, including: receiving, at at least one computing device, one or more datasets corresponding to a desired material; creating, using at least two machine learning models associated with the at least one computing device, a new dataset for the desired material, where the at least two machine learning models are trained, using at least semi-supervised learning, based on the one or more datasets, to model properties of the desired material; and outputting, using the at least one computing device, a prediction including the desired material.

In some aspects, the techniques described herein relate to a method, where the at least two machine learning models include at least one of machine learning interatomic potential (MLIP), machine learned charge density (MLCD), or machine learning property predictor (MLProp). In some aspects, the techniques described herein relate to a method, where the at least two machine learning models include at least two of machine learning interatomic potential (MLIP), machine learned charge density (MLCD), or machine learning property predictor (MLProp).

In some aspects, the techniques described herein relate to a method, where the at least two machine learning models include quantum data or Quantum Probabilistic Machine Learning. In some aspects, the techniques described herein relate to a method, where the at least two machine learning models are predictive machine learning models. In some aspects, the techniques described herein relate to a method, further including evaluating the desired material using uncertainty-driven active learning.

In some aspects, the techniques described herein relate to a method, further including: outputting a trained dataset based on the semi-supervised learning; using the trained dataset to create a second new dataset for the desired material; predicting, using the at least two machine learning models, the desired material based on the second new dataset; generating, using the prediction, a proposed atomic structure; refining the proposed atomic structure by adjusting the structure to maximize stability; evaluating the proposed atomic structure using uncertainty-driven active learning; determining that the proposed atomic structure is below an error threshold; and validating the proposed atomic structure via density functional theory.

In some aspects, the techniques described herein relate to a method, further including: outputting, using the at least one computing device, a generative model; and determining, using the at least two machine learning models associated with the at least one computing device, an accuracy of the generative model. In some aspects, the techniques described herein relate to a method, where the generative model is outputted within an active learning computing environment. In some aspects, the techniques described herein relate to a method, where the prediction is outputted based on the generative model. In some aspects, the techniques described herein relate to a method, where the prediction includes iteratively reassessing the dataset for the desired material with the properties of the desired material until the properties of the desired material satisfies a predetermined threshold.

In some aspects, the techniques described herein relate to a method, where the prediction is outputted only when the generative model satisfies the predetermined threshold. In some aspects, the techniques described herein relate to a method, where the prediction is outputted in fewer computing cycles compared to a conventional computation of the one or more datasets. In some aspects, the techniques described herein relate to a method, where the desired material and properties of the desired material are received and analyzed using one or more Density Functional Theory (DFT) models.

In some aspects, the techniques described herein relate to a method, where at least one of: the creating integrates classical machine learning and quantum machine learning; the creating includes using experiment via Bayesian Optimization (BO) to predict the desired material; or the creating uses uncertainty-driven active learning cycles to create the new dataset for the desired material.

In some aspects, the techniques described herein relate to a method, where the new dataset for the desired material includes at least two of electronic properties, charge density, force vectors, interatomic potential, or a scalar value property associated with the desired material. In some aspects, the techniques described herein relate to a method, further including synthesizing the desired material based on the prediction. In some aspects, the techniques described herein relate to a method, where the at least semi-supervised learning uses uncertainty-driven active learning. In some aspects, the techniques described herein relate to a method, where the at least two machine learning models are configured such that the prediction can be outputted in fewer processing cycles compared to conventional computing systems. In some aspects, the techniques described herein relate to a method, where the at least two machine learning models are used in parallel. In some aspects, the techniques described herein relate to a method, where the at least two machine learning models are used in serial.

In some aspects, the techniques described herein relate to a system, including: a non-transitory memory storing instructions; and one or more processors in communication with the non-transitory memory, where the one or more processors execute the instructions to: receive, at at least one computing device, one or more datasets corresponding to a desired material; create, using at least two machine learning models associated with the at least one computing device, a new dataset for the desired material, where the at least two machine learning models are trained, using at least semi-supervised learning, based on the one or more datasets, to model properties of the desired material; and output, using the at least one computing device, a prediction including the desired material.

In some aspects, the techniques described herein relate to a method, including: predicting, using at least one processor, using at least two machine learning models, a new material with desired properties by: predicting the new material using at least two machine learning models, where the at least two machine learning models are trained based on the desired properties; analyzing the prediction of the new material via the at least two machine learning models; and outputting the prediction once the prediction exceeds a predetermined threshold associated with the at least two machine learning models.

In some aspects, the techniques described herein relate to a method, including: creating an active learning cycle, using at least two machine learning models, by: generating training data based on property data, iteratively reprocessing the property data and the training data until a predetermined threshold is met, where the predetermined threshold includes a set of characteristics, and once the predetermined threshold is met, generating a predictive model; generating a machine learning model using the predictive model; and applying the machine learning model to predict a new material having the characteristics.

The use of the terms “a” and “an” and “the” and similar referents in the context of describing the subject matter (particularly in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Furthermore, the foregoing description is for the purpose of illustration only, and not for the purpose of limitation, as the scope of protection sought is defined by the claims as set forth hereinafter together with any equivalents thereof entitled to. The use of any and all examples, or exemplary language (e.g., “such as”) provided herein, is intended merely to better illustrate the subject matter and does not pose a limitation on the scope of the subject matter unless otherwise claimed. The use of the term “based on” and other like phrases indicating a condition for bringing about a result, both in the claims and in the written description, is not intended to foreclose any other conditions that bring about that result. No language in the specification should be construed as indicating any non-claimed element as essential to the practice of the invention as claimed.

The embodiments described herein included the one or more modes known to the inventor for carrying out the claimed subject matter. Of course, variations of those embodiments will become apparent to those of ordinary skill in the art upon reading the foregoing description. The inventor expects skilled artisans to employ such variations as appropriate, and the inventor intends for the claimed subject matter to be practiced otherwise than as specifically described herein. Accordingly, this claimed subject matter includes all modifications and equivalents of the subject matter recited in the claims appended hereto as permitted by applicable law. Moreover, any combination of the above-described elements in all possible variations thereof is encompassed unless otherwise indicated herein or otherwise clearly contradicted by context.

1. A method for improving materials discovery and development, comprising:
receiving, on at least one computing device, one or more datasets corresponding to a material having preconfigured properties;
creating, using at least two machine learning models associated with the at least one computing device, a new dataset for the material having preconfigured properties, wherein the at least two machine learning models are trained using fully or partially semi-supervised learning based on the one or more datasets, to model properties of the material having preconfigured properties, wherein the creating reduces computational resources compared to conventional materials discovery methods; and
outputting, using the at least one computing device, a prediction comprising the material having preconfigured properties, wherein the prediction comprises specifications for synthesizing and characterizing new materials.

2. The method of claim 1, wherein the creating uses at least three machine learning models comprising machine learning interatomic potential (MLIP), machine learning charge density (MLCD), and machine learning property predictor (MLProp).

3. The method of claim 1, wherein the at least two machine learning models include at least two of machine learning interatomic potential (MLIP), machine learned charge density (MLCD), or machine learning property predictor (MLProp).

4. The method of claim 1, wherein the at least two machine learning models include quantum computer-generated data and Quantum Probabilistic Machine Learning.

5. The method of claim 1, wherein the at least two machine learning models are predictive machine learning models.

6. The method of claim 1, further comprising evaluating the material having preconfigured properties using uncertainty-driven active learning.

7. The method of claim 1, further comprising: training two or more predictive machine learning models on the one or more datasets; using the trained predictive models to create a second larger dataset; generating, using a generative prediction machine learning model trained on the second larger dataset, a proposed atomic structure; refining the proposed atomic structure by adjusting the structure to maximize stability; evaluating the proposed atomic structure using uncertainty-driven active learning; determining that the proposed atomic structure is below an error threshold; and validating the proposed atomic structure via density functional theory.

8. The method of claim 1, further comprising: outputting, using the at least one computing device, a generative model; and determining, using the at least two machine learning models associated with the at least one computing device, an accuracy of the generative model.

9. The method of claim 8, wherein the generative model is outputted within an active learning computing environment.

10. The method of claim 8, wherein the prediction is outputted based on the generative model.

11. The method of claim 10, wherein the prediction includes iteratively reassessing the dataset for the material having preconfigured properties until the preconfigured properties satisfy a predetermined threshold.

12. The method of claim 11, wherein the prediction is outputted only when the generative model satisfies the predetermined threshold.

13. The method of claim 1, wherein the prediction is outputted in fewer computing cycles compared to a conventional computation of the one or more datasets.

14. The method of claim 1, wherein the material having preconfigured properties are received and analyzed using one or more Density Functional Theory (DFT) models.

15. The method of claim 1, wherein at least one of: the creating integrates classical machine learning and quantum machine learning; the creating includes using Bayesian Optimization (BO) to generate data for the new dataset for the desired material; or the creating uses uncertainty-driven active learning cycles to create the new dataset for the material having preconfigured properties.

16. The method of claim 1, wherein the new dataset for the material having preconfigured properties includes at least two of electronic properties, charge density, force vectors, interatomic potential, or a scalar value property associated with the material having preconfigured properties.

17. The method of claim 1, further comprising synthesizing the material having preconfigured properties based on the prediction.

18. The method of claim 1, wherein the at least semi-supervised learning uses uncertainty-driven active learning.

19. The method of claim 1, wherein the at least two machine learning models are configured such that the prediction can be outputted in fewer processing cycles compared to conventional computing systems.

20. The method of claim 1, wherein the at least two machine learning models are used in parallel.

21. The method of claim 1, wherein the at least two machine learning models are used in serial.

22. A system for improving materials discovery and development, comprising:
a non-transitory memory storing instructions; and one or more processors in communication with the non-transitory memory, wherein the one or more processors execute the instructions to:
receive, on at least one computing device, one or more datasets corresponding to a material having preconfigured properties;
create, using at least two machine learning models associated with the at least one computing device, a new dataset for the material having preconfigured properties, wherein the at least two machine learning models are trained using fully or partially semi-supervised learning based on the one or more datasets, to model properties of the material having preconfigured properties, wherein the creating reduces computational resources compared to conventional materials discovery methods; and
output, using the at least one computing device, a prediction comprising the material having preconfigured properties, wherein the prediction comprises specifications for synthesizing and characterizing new materials.

23. A computer program product for improving materials discovery and development comprising computer executable instructions stored on a non-transitory computer readable medium that when executed by a processor instruct the processor to:
receive, on at least one computing device, one or more datasets corresponding to a material having preconfigured properties;
create, using at least two machine learning models associated with the at least one computing device, a new dataset for the material having preconfigured properties, wherein the at least two machine learning models are trained using fully or partially semi-supervised learning based on the one or more datasets, to model properties of the material having preconfigured properties, wherein the creating reduces computational resources compared to conventional materials discovery methods; and
output, using the at least one computing device, a prediction comprising the material having preconfigured properties, wherein the prediction comprises specifications for synthesizing and characterizing new materials.

24. A method, comprising: creating an active learning cycle, using at least two machine learning models, by: generating training data based on property data, iteratively reprocessing the property data and the training data until a predetermined threshold is met, wherein the predetermined threshold includes a set of characteristics, and once the predetermined threshold is met, generating a predictive model; generating a machine learning model using the predictive model; and applying the machine learning model to predict a new material having the characteristics.
